SEQUENCE LISTING (56 sequences)

SEQ ID NO: 1 - skipped

SEQ ID NO: 2 - protein, 121 aa, synthetic construct
EVQLLESGGGLVQPGGSLRLSCAASEFTFGSCVMTWVRQAPGKGLEWVSVISGSGGSTYYADSVKGRFTISRDNSKNTLYLQMNSLRAEDTAVYYCAKTPWWLRSPFFDYWGQGTLVTVSS

SEQ ID NO: 3 - skipped

SEQ ID NO: 4 - protein, 121 aa, synthetic construct
QLLLQESGPGLVKPSETLSLTCTVSGGSLSSSFSYWVWIRQPPGKGLEWIGTIYYSGNTYYNPSLKSRLTISVDTSKNHFSLNLSSVTAADTAVYYCARRAGILTGYLDSWGQGTLVTVSS

SEQ ID NO: 5 - skipped

SEQ ID NO: 6 - protein, 121 aa, synthetic construct
QLLLQESGPGLVKPSETLSLTCTVSGGSLSSSFSYWVWIRQPPGKGLEWIGTIYYSGNTYYNPSLKSRLTISVDTSKNHFSLKLSSVTAADTAVYYCARRAGILTGYLDSWGQGTLVTVSS

SEQ ID NO: 7 - skipped

SEQ ID NO: 8 - protein, 118 aa, synthetic construct
QVQLVQSGAEVKKPGASVKVSCKASGYTFTGCYMHWVRQAPGQGLEWMGRINPNSGGTNYAQKFQGRVTMTRDTSISTAYMDLSRLRSDDTAVYYCARDYSGWSFDYWGQGTLVTVSS

SEQ ID NO: 9 - skipped

SEQ ID NO: 10 - protein, 123 aa, synthetic construct
QLQLQESGPGLVKPSETLSLTCTVSGGSISSSSYYWGWIRQPPGKGLEWIGNIYYSGSTYYNPSLMSRVTISVDTSKNQFSLKLSSVTAADTAVYYCARQLSYNWNDRLFDYWGQGTLVTVSS

SEQ ID NO: 11 - skipped

SEQ ID NO: 12 - protein, 119 aa, synthetic construct
SYVLTQPPSVSVAPGQTARITCGGNNIGSTSVHWYQQKPGQAPVLVVHDDSDRPSGIPERFSGSNSGNTATLTISRVEAGDEADYYCQVRDSSSDHVIFGGGTKLTVLGQPKAAPSVTL

SEQ ID NO: 13 - skipped

SEQ ID NO: 14 - protein, 119 aa, synthetic construct
SYVLTQSPSVSVAPGQTARITCGGNNIGSKSVHWYQQKPGQAPVLVVYDDSDRPSGIPERFSASNSGNTATLTISRVEAGDEADYYCQVWDSSSDHVVFGGGTKLTVLGQPKAAPSVTL

SEQ ID NO: 15 - skipped

SEQ ID NO: 16 - protein, 119 aa, synthetic construct; identical to SEQ ID NO: 14

SEQ ID NO: 17 - skipped

SEQ ID NO: 18 - protein, 117 aa, synthetic construct
DIQMTQSPSSLSASVGDRVTITCRASQGISNYLAWFQQKPGKAPKSLIYAASSLQSGVPSKFSGSGSGTDFTLTISSLQSEDFATYYCQQYDSYPPTFGGGTKVEIKRTVAAPSVFI

SEQ ID NO: 19 - skipped

SEQ ID NO: 20 - protein, 117 aa, synthetic construct
SYELTQPLSVSVALGQTARITCGGNNIGSKNVHWYQQKPGQAPVLVIYRDSNRPSGIPERFSGSNSGNTATLTISRAQAGDEADYYCQVWDSSTVVFGGGTKLTVLGQPKAAPSVTL

SEQ ID NO: 21 - protein, 5 aa, synthetic construct
SCVMT

SEQ ID NO: 22 - protein, 17 aa, synthetic construct
VISGSGGSTYYADSVKG

SEQ ID NO: 23 - protein, 12 aa, synthetic construct
TPWWLRSPFFDY

SEQ ID NO: 24 - protein, 11 aa, synthetic construct
GGNNIGSTSVH

SEQ ID NO: 25 - protein, 7 aa, synthetic construct
DDSDRPS

SEQ ID NO: 26 - protein, 10 aa, synthetic construct
VRDSSSDHVI

SEQ ID NO: 27 - protein, 5 aa, synthetic construct
FSYWV

SEQ ID NO: 28 - protein, 16 aa, synthetic construct
TIYYSGNTYYNPSLKS

SEQ ID NO: 29 - protein, 11 aa, synthetic construct
RAGILTGYLDS

SEQ ID NO: 30 - protein, 11 aa, synthetic construct
GGNNIGSKSVH

SEQ ID NO: 31 - protein, 7 aa, synthetic construct
DDSDRPS

SEQ ID NO: 32 - protein, 11 aa, synthetic construct
QVWDSSSDHVV

SEQ ID NO: 33 - protein, 5 aa, synthetic construct
GCYMH

SEQ ID NO: 34 - protein, 17 aa, synthetic construct
RINPNSGGTNYAQKFQG

SEQ ID NO: 35 - protein, 9 aa, synthetic construct
DYSGWSFDY

SEQ ID NO: 36 - protein, 11 aa, synthetic construct
RASQGISNYLA

SEQ ID NO: 37 - protein, 7 aa, synthetic construct
AASSLQS

SEQ ID NO: 38 - protein, 10 aa, synthetic construct
QQYDSYPPTF

SEQ ID NO: 39 - protein, 5 aa, synthetic construct
SYYWG

SEQ ID NO: 40 - protein, 16 aa, synthetic construct
NIYYSGSTYYNPSLMS

SEQ ID NO: 41 - protein, 13 aa, synthetic construct
QLSYNWNDRLFDY

SEQ ID NO: 42 - protein, 11 aa, synthetic construct
GGNNIGSKNVH

SEQ ID NO: 43 - protein, 7 aa, synthetic construct
RDSNRPS

SEQ ID NO: 44 - protein, 9 aa, synthetic construct
QVWDSSTVV

SEQ ID NO: 45 - skipped

SEQ ID NO: 46 - protein, 450 aa, synthetic construct
QLLLQESGPGLVKPSETLSLTCTVSGGSLSSSFSYWVWIRQPPGKGLEWIGTIYYSGNTYYNPSLKSRLTISVDTSKNHFSLKLSSVTAADTAVYYCARRAGILTGYLDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 47 - skipped

SEQ ID NO: 48 - protein, 214 aa, synthetic construct
SYVLTQSPSVSVAPGQTARITCGGNNIGSKSVHWYQQKPGQAPVLVVYDDSDRPSGIPERFSASNSGNTATLTISRVEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 49 - protein, 5 aa, synthetic construct
SSVMT

SEQ ID NO: 50 - protein, 11 aa, synthetic construct
GGSNIGSTSVH

SEQ ID NO: 51 - protein, 121 aa, synthetic construct
EVQLLESGGGLVQPGGSLRLSCAASEFTFGSSVMTWVRQAPGKGLEWVSVISGSGGSTYYADSVKGRFTISRDNSKNTLYLQMNSLRAEDTAVYYCAKTPWWLRSPFFDYWGQGTLVTVSS

SEQ ID NO: 52 - protein, 119 aa, synthetic construct
SYVLTQPPSVSVAPGQTARITCGGSNIGSTSVHWYQQKPGQAPVLVVHDDSDRPSGIPERFSGSNSGNTATLTISRVEAGDEADYYCQVRDSSSDHVIFGGGTKLTVLGQPKAAPSVTL

SEQ ID NO: 53 - protein, 450 aa, synthetic construct
EVQLLESGGGLVQPGGSLRLSCAASEFTFGSSVMTWVRQAPGKGLEWVSVISGSGGSTYYADSVKGRFTISRDNSKNTLYLQMNSLRAEDTAVYYCAKTPWWLRSPFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 54 - protein, 200 aa, synthetic construct
SYVLTQPPSVSVAPGQTARITCGGSNIGSTSVHWYQQKPGQAPVLVVHDDSDRPSGIPERFSGSNSGNTATLTISRVEAGDEADYYCQVRDSSSDHVIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHE

SEQ ID NO: 55 - protein, 447 aa, synthetic construct
EVQLLESGGGLVQPGGSLRLSCAASEFTFGSSVMTWVRQAPGKGLEWVSVISGSGGSTYYADSVKGRFTISRDNSKNTLYLQMNSLRAEDTAVYYCAKTPWWLRSPFFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 56 - protein, 214 aa, synthetic construct
SYVLTQPPSVSVAPGQTARITCGGSNIGSTSVHWYQQKPGQAPVLVVHDDSDRPSGIPERFSGSNSGNTATLTISRVEAGDEADYYCQVRDSSSDHVIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2026/0250392 A1
(43) Pub. Date: Aug. 27, 2026

(54) HIGH CONCENTRATION PROTEIN FORMULATIONS WITH POLYSORBATE EXCIPIENTS AND METHODS OF MAKING THE SAME

(71) Applicant: Immunovant Sciences GmbH, Base (CH)

(72) Inventor: Pooja Balchandani Mehta, Cary, NC (US)

(73) Assignee: Immunovant Sciences GmbH, Base (CH)

(21) Appl. No.: 19/162,698

(22) PCT Filed: Mar. 7, 2024
(86) PCT No.: PCT/EP2024/055958

§ 371 Date: Sep. 5, 2025

Related U.S. Application Data
(60) Provisional application No. 63/489,132, filed on Mar. 8, 2023.

(51) Int. Cl. C07K 16/28; A61K 47/26; C07K 1/22; C07K 1/34

(52) U.S. Cl. CPC C07K 16/283; A61K 47/26; C07K 1/22; C07K 1/34; C07K 2317/21; C07K 2317/24

(57) ABSTRACT

The present disclosure relates to formulations comprising a protein comprising an Fc region (e.g., an antibody) and a surfactant such as a polysorbate (e.g., Polysorbate 20), and methods of purifying such proteins that improve stability of the surfactant such as a polysorbate in such formulations.

### CROSS-REFERENCE TO RELATED APPLICATIONS

The present application claims the benefit of U.S. Provisional Patent Application No. 63/489,132 filed Mar. 8, 2023, which is incorporated herein by reference in its entirety.

### INCORPORATION-BY-REFERENCE OF SEQUENCE LISTING

The Sequence Listing XML associated with this application is provided electronically in XML file format and is hereby incorporated by reference into the specification. The name of the XML file containing the Sequence Listing XML is “MUNO-012_00US_SeqList_ST26”. The XML file is 44,393 bytes, created on Feb. 28, 2023, and is being submitted electronically herewith.

### FIELD

The present disclosure relates to formulations comprising a protein having an Fc region and one or more excipients comprising a surfactant such as a polysorbate, and methods of purifying proteins having an Fc region that improve stability of such surfactants in such formulations.

### BACKGROUND

A surfactant (e.g., Polysorbate 20, Polysorbate 80) is usually required to be added to liquid protein formulations at drug substance or drug product stage, specially at high protein concentrations (>50 g/L or >100 g/L), to stabilize the product under various thermal, mechanical, and interfacial shear conditions (see Khan et al., Eur. J. of Pharm. and Biopharmaceutics 97: 60, 2015, Wang et al., Antibody Therapeutics 4:262-273, 2021). This stabilizing effect of the surfactant protects the antibody or protein formulation against thermal, mechanical, and interfacial shear stress as the material goes through drug product manufacturing (fill/finish), packaging, storage, handling & distribution. Degradation of the surfactant may lead to loss of this protective effect against stress conditions leading to undesired outcomes like particle formation, impact on purity and other quality attributes of drug substance or drug product (see Liu, Lu et al, J Pharm Sci 102:2460, 2013). Hence, surfactant (e.g., Polysorbate 20 (PS20) or Polysorbate 80 (PS80)) stability needs to be improved at the drug substance stage to prevent the molecule from shear stress of freeze thaw and drug product filling, packaging, transport, and storage.

### SUMMARY

In one aspect, provided herein is a formulation comprising (i) a protein having an Fc region at a concentration of higher than 50 g/L, and (ii) one or more excipients comprising a polysorbate, wherein the concentration of the polysorbate remains within 30% of the starting concentration of the polysorbate after storage for a period of time of at least 1 day and/or wherein the presence of a polysorbate degradation product does not increase by more than 2 times of the starting concentration of the degradation product after storage for a period of time of at least 1 day. In some embodiments, the concentration of higher than 50 mg/ml is a concentration equal to or higher than 75 g/L or 100 g/L. In some embodiments, the concentration of higher than 100 mg/ml is a concentration equal to or higher than 150 g/L, 170 g/L, or 200 g/L.

In some embodiments, the protein having an Fc region is an antibody. In some embodiments, the antibody is a humanized or fully human monoclonal antibody. In some embodiments, the antibody is an IgG1 antibody. In some embodiments, the antibody is an anti-FcRn antibody that specifically binds to FcRn. In some embodiments, the anti-FcRn antibody is a fully human monoclonal IgG1 antibody. In some embodiments, the anti-FcRn antibody comprises

(a) (i) three heavy chain CDR amino acid sequences of SEQ ID No: 27 (HCDR1), SEQ ID No: 28 (HCDR2), SEQ ID No: 29 (HCDR3); and (ii) three light chain CDR amino acid sequences of SEQ ID No: 30 (LCDR1), SEQ ID No: 31 (LCDR2), SEQ ID No: 32 (LCDR3); or (b) (i) three heavy chain CDR amino acid sequences of SEQ ID No: 49 (HCDR1), SEQ ID No: 22 (HCDR2), SEQ ID No: 23 (HCDR3); and (ii) three light chain CDR amino acid sequences of SEQ ID No: 50 (LCDR1), SEQ ID No: 25 (LCDR2), SEQ ID No: 26 (LCDR3). In some embodiments, the anti-FcRn antibody comprises (a) a heavy chain variable region amino acid sequence of SEQ ID No: 6 and a light chain variable region amino acid sequence of SEQ ID No: 16; or (b) a heavy chain amino acid sequence of SEQ ID No: 51; and a light chain amino acid sequence of SEQ ID No: 52.

In some embodiments, the polysorbate is Polysorbate 20. In some embodiments, the polysorbate is Polysorbate 80. In some embodiments, the polysorbate is at a concentration from about 0.002% to 0.2%. In some embodiments, the polysorbate is at a concentration from 0.01 to 0.05%. In some embodiments, the pharmaceutical formulation comprises at least or about 170 g/L of the anti-FcRn antibody, 0.02% Polysorbate 20, 100 mM L-Histidine/Histidine HCl and 100 mM L-Arginine HCl in water at pH 6.0.

In some embodiments, the concentration of the polysorbate remains within 20% of the starting concentration of the polysorbate after storage. In some embodiments, the concentration of the polysorbate remains within 10% of the starting concentration of the polysorbate after storage. In some embodiments, the concentration of the polysorbate remains within 5% of the starting concentration of the polysorbate after storage. In some embodiments, the polysorbate is Polysorbate 20, and the degradation product is a non-esterified species of Polysorbate 20. In some embodiments, the non-esterified species of Polysorbate 20 is Sorbitan or Isosorbide. In some embodiments, the polysorbate is Polysorbate 20, and Polysorbate 20 degradation product is Lauric acid. In some embodiments, the polysorbate is Polysorbate 80, and Polysorbate 80 degradation product is Oleic acid.

In some embodiments, the presence of a polysorbate degradation product does not increase by more than 50% of the starting concentration of the degradation product after storage. In some embodiments, the presence of a polysorbate degradation product does not increase by more than 25% of the starting concentration of the degradation product after storage. In some embodiments, the presence of a polysorbate degradation product does not increase by more than 10% of the starting concentration of the degradation product after storage. In some embodiments, the concentration of polyester species of the polysorbate remain within 10% or 20% of the starting concentration of the polyester species after storage for a period of time of at least 1 day. In some embodiments, the concentration of monoester species of the polysorbate remain within 10% or 20% of the starting concentration of the monoester species after storage for a period of time of at least 1 day. In some embodiments, the storage for a period of time of at least 1 day is storage for 1, 3 or 7 days. In some embodiments, the storage for a period of time of at least 1 day is storage for about or more than 14 or 28 days. In some embodiments, the storage for a period of time of at least 1 days is storge for about or more than 8 weeks or 12 weeks. In some embodiments, the storage for a period of time of at least 1 days is storage for about or more than 1 year. In some embodiments, the storage is at 2-8° C. In some embodiments, the storage is at 25° C. In some embodiments, the storage is at 40° C.

In some embodiments, the formulation has no or substantially no cell culture host cell proteins having lipase and/or hydrolase activity. In some embodiments, the formulation is a pharmaceutical formulation, and wherein the one or more excipients are pharmaceutically acceptable. In some embodiments, the protein having an Fc region has been purified from cell culture by an Fc-binding affinity resin chromatography. In some embodiments, the Fc-binding affinity resin chromatography is a Protein A chromatography column or a Protein G chromatography column.

In another aspect, provided herein is a method of purifying a protein having an Fc region from harvested cell culture, the method comprising: (i) loading the harvested cell culture comprising the protein having an Fc region on to an Fc-binding affinity resin chromatography column, (ii) washing the column one, two, three or more times using one or more wash buffers, wherein at least one wash buffer comprises a pH of greater than 9, and (iii) eluting the protein having an Fc region from the column; optionally, wherein the method is for making a high concentration formulation of the protein with one or more excipients comprising a polysorbate. In some embodiments, the method further comprises, after step (iii), formulating the protein from step (iii) into a formulation comprising adding one or more excipients, wherein the concentration of the protein in the formulation is higher than 50 g/L, and wherein the one or more excipients comprise a polysorbate.

In some embodiments, the polysorbate is Polysorbate 20. In some embodiments, the polysorbate is Polysorbate 80. In some embodiments, the polysorbate in the formulation is at a concentration from about 0.002% to 0.2%. In some embodiments, the polysorbate in the formulation is at a concentration from 0.01 to 0.05%.

In some embodiments, the Fc-binding affinity resin chromatography column is a Protein A chromatography column. In some embodiments, the Fc-binding affinity resin chromatography column is a Protein G chromatography column. In some embodiments, the Fc-binding affinity resin chromatography column comprises a genetically engineered Fc binding protein. In some embodiments, the Fc-binding affinity resin chromatography column load ratio in step (i) is 35 g/L or less. In some embodiments, the Fc-binding affinity resin chromatography column load ratio in step (i) is 25 g/L or less.

In some embodiments, the at least one wash buffer comprises a pH of at least or more than 10. In some embodiments, the at least one wash buffer comprises a pH of about 10. In some embodiments, the at least one wash buffer comprises a pH of at least or more than 10.5. In some embodiments, the at least one wash buffer comprises a pH of about 10.5. In some embodiments, the at least one wash buffer comprises a pH of at least 11 or about 11.

In some embodiments, the at least one wash buffer comprises 1M Sodium Chloride or higher concentration of Sodium Chloride. In some embodiments, the at least one wash buffer comprises concentration of Sodium Chloride that is less than 1M or less than 0.5M. In some embodiments, the at least one wash buffer comprises arginine. In some embodiments, the at least one wash buffer does not comprise arginine. In some embodiments, the at least one wash buffer comprises sodium phosphate or sodium carbonate. In some embodiments, the at least one wash buffer comprises 50 mM sodium carbonate.

In some embodiments, the concentration of the protein having an Fc region in the formulation is about or higher than 100 g/L. In some embodiments, the concentration of the protein having an Fc region in the formulation is about or higher than 150 g/L, 170 g/L, or 200 g/L.

In some embodiments, the formulation is a pharmaceutical formulation.

In some embodiments, the protein having an Fc region is an antibody. In some embodiments, the antibody is a humanized or human monoclonal antibody. In some embodiments, the antibody is an IgG1 antibody. In some embodiments, the antibody is an anti-FcRn antibody that specifically binds to FcRn. In some embodiments, the anti-FcRn antibody is a fully human monoclonal IgG1 antibody. In some embodiments, the anti-FcRn antibody comprises (a) (i) three heavy chain CDR amino acid sequences of SEQ ID No: 27 (HCDR1), SEQ ID No: 28 (HCDR2), SEQ ID No: 29 (HCDR3); and (ii) three light chain CDR amino acid sequences of SEQ ID No: 30 (LCDR1), SEQ ID No: 31 (LCDR2), SEQ ID No: 32 (LCDR3); or (b) (i) three heavy chain CDR amino acid sequences of SEQ ID No: 49 (HCDR1), SEQ ID No: 22 (HCDR2), SEQ ID No: 23 (HCDR3); and (ii) three light chain CDR amino acid sequences of SEQ ID No: 50 (LCDR1), SEQ ID No: 25 (LCDR2), SEQ ID No: 26 (LCDR3). In some embodiments, the anti-FcRn antibody comprises (a) a heavy chain variable region amino acid sequence of SEQ ID No: 6 and a light chain variable region amino acid sequence of SEQ ID No: 16; or (b) a heavy chain amino acid sequence of SEQ ID No: 51; and a light chain amino acid sequence of SEQ ID No: 52.

In some embodiments, the harvested cell culture is a cell culture fluid harvested from host cells used for production of the protein having an Fc region, optionally wherein the production is by introducing a vector comprising the protein having an Fc region into the host cells and culturing the host cells under conditions suitable for expression of the protein, optionally wherein the host cells are mammalian cells suitable for the production. In some embodiments, the host cells are CHO cells. In some embodiments, the host cells are harvested by centrifugation, optionally followed by filtration. In some embodiments, after step (iii), the eluate, comprising the protein having an Fc region, is subjected to filtration. In some embodiments, after step (iii), the eluate, comprising the protein having an Fc region, is subjected to low pH viral inactivation.

In some embodiments, the eluate, comprising the protein having an Fc region, is subjected to anion exchange (AEX) chromatography and/or cation exchange (CEX) chromatography. In some embodiments, after step (iii) and optionally after one, two, three or all of the steps specified in any of the above embodiments, the eluate, comprising the protein having an Fc region, is subjected to concentration. In some embodiments, the protein having an Fc region is an anti-FcRn antibody that specifically binds to FcRn, and wherein the formulation is a pharmaceutical formulation comprising at least or about 170 g/L of the anti-FcRn antibody, 0.02% Polysorbate 20, 100 mM L-Histidine/Histidine HCl and 100 mM L-Arginine HCl in water at pH 6.0.

In another aspect, provided herein is a formulation produced using a method described herein. In some embodiments, the polysorbate comprises Polysorbate 20 (PS20) or Polysorbate 80 (PS80), and wherein the concentration of the PS20 or PS80 remains within 30% of the starting concentration of the PS20 or PS80 after storage for a period of time of at least 1 day and/or wherein the presence of a PS20 or PS80 degradation product does not increase by more than 2 times of the starting concentration of the degradation product after storage for a period of time of at least 1 day. In some embodiments, the concentration of the PS20 or PS80 remains after storage within 20% of the starting concentration of the PS20 or PS80. In some embodiments, the concentration of the PS20 or PS80 remains after storage within 10% of the starting concentration of the PS20 or PS80. In some embodiments, the concentration of the PS20 or PS80 remains after storage within 5% of the starting concentration of the PS20 or PS80. In some embodiments, the PS20 or PS80 degradation product is a fatty acid ester. In some embodiments, the PS20 degradation product is a non-esterified species of PS20. In some embodiments, the non-esterified species of PS20 is Sorbitan or Isosorbide. In some embodiments, the PS20 degradation product is Lauric acid. In some embodiments, the PS80 degradation product is Oleic acid. In some embodiments, the presence of a PS20 or PS80 degradation product does not increase by more than 50% of the starting concentration of the degradation product after storage. In some embodiments, the presence of a PS20 or PS80 degradation product does not increase by more than 25% of the starting concentration of the degradation product after storage. In some embodiments, the presence of a PS20 or PS80 degradation product does not increase by more than 10% of the starting concentration of the degradation product after storage. In some embodiments, the concentration of polyester species of PS20 or PS80 remain within 10% or 20% of the starting concentration of the polyester species after storage for a period of time of at least 1 day. In some embodiments, the concentration of monoester species of PS20 or PS80 remain within 10% or 20% of the starting concentration of the monoester species after storage for a period of time of at least 1 day. In some embodiments, storage for a period of time of at least 1 day is storage for 1, 3 or 7 days. In some embodiments, storage for a period of time of at least 1 day is storage for about or more than 14 or 28 days. In some embodiments, storage for a period of time of at least 1 days is storge for about or more than 8 weeks or 12 weeks. In some embodiments, storage for a period of time of at least 1 days is storage for about or more than 1 year. In some embodiments, the storage is at 2-8° C. In some embodiments, the storage is at 25° C. In some embodiments, the storage is at 40° C. In some embodiments, the formulation has no or substantially no cell culture host cell proteins having lipase, esterase and/or hydrolase activity.

### BRIEF DESCRIPTION OF FIGURES

FIGS. 1A-1D show the structures of Polysorbate 20, Polysorbate 80, lauric acid, and oleic acid, respectively.

FIG. 2A is a flow diagram showing a platform cell culture (upstream) process for a protein having an Fc region (e.g., anti-FcRn antibody). FIG. 2B is a flow diagram showing a platform purification (downstream) process for a protein having an Fc region (e.g., anti-FcRn antibody).

FIG. 3 shows PS20 stability in drug substance using an established manufacturing process. Data was collected by spiking 200 μg/ml PS-20 in UFDF material.

FIG. 4 shows PS20 stability of placebo (formulation buffer); placebo target concentration is 220 μg/ml.

FIG. 5 shows results of an in-process intermediate spiking study for drug substance: PS-20 degradation kinetics for Protein A pool, VIN Pool, AEX Pool, CEX Pool and UFDF Pool at 40° C.

FIG. 6 shows a flow diagram of the Protein A affinity chromatography unit operation process.

FIG. 7 shows PS20 degradation over time when incubated with Protein A pool (intermediate) with several different wash conditions.

FIG. 8 shows residual host cell protein (HCP) levels in Protein A pool by wash condition.

FIG. 9 shows design of experiment, Prediction Profiler using JMP software.

FIG. 10 shows a variability chart for residual HCP concentrations by buffer condition.

FIG. 11 shows a variability chart for PS20 Day 14 concentrations by buffer condition.

FIG. 12 shows a variability chart for PS20 Day 30 concentrations by buffer condition.

FIG. 13 shows a variability chart for SEC-HMW (%) concentrations by buffer condition.

FIG. 14 shows the Prediction Profile of PS20 stability at Day 7 and Day 14 with parameter set at center point.

FIG. 15 shows a variability chart for high molecular weight (HMW) (by size exclusion chromatography) by wash 2 buffer pH.

FIG. 16 shows a variability chart for PS20 T14 by wash 2 buffer pH and load ratio.

FIG. 17 shows bulk drug substance (BDS) stability at storage (−70° C.), accelerated (2-8° C.) and stressed (25° C.) temperature.

FIG. 18 shows a comparison of Protein A eluate stability between the old and new wash conditions.

FIG. 19 shows a comparison between ultrafiltration/diafiltration (UFDF) pool stability between the old and new wash conditions.

FIG. 20 shows a comparison of BDS stability between old and new wash conditions.

FIG. 21 shows the poly-ester profile for PS20 in Protein A eluate at 40° C.

FIG. 22 shows the mono-ester profile for PS20 in Protein A eluate at 40° C.

FIG. 23 shows the non-ester profile for PS20 in Protein A eluate at 40° C.

FIG. 24 shows the poly-ester profile for PS20 in BDS at 5° C. and 40° C.

FIG. 25 shows the monoester profile for PS20 in BDS at 5° C. and 40° C.

FIG. 26 shows the non-ester profile for PS20 in BDS at 5° C. and 40° C.

FIG. 27 shows the lauric acid profile for PS20 in BDS at 5° C. and 40° C.

FIG. 28 shows the non-ester profile by LC/MS for intermediate pool stability at 40° C.

FIG. 29 shows the mono-ester profile by LC/MS for intermediate pool stability at 40° C.

FIG. 30 shows the poly-ester profile by LC/MS for intermediate pool stability at 40° C.

FIG. 31 shows ester profile by LC/MS for drug substance stability at 5° C.

### DETAILED DESCRIPTION

Provided herein are formulations comprising a protein having an Fc region and a surfactant such as a polysorbate (e.g., PS20 or PS80), with an improved stability of the surfactant, and methods of producing such formulations. Without wishing to be bound by theory, it is believed that lipase-like activity that co-purifies with a protein having an Fc region is responsible for the degradation of the polysorbate at refrigerated or elevated temperatures. Accordingly, provided herein are methods for purification of proteins having an Fc region that allow removal of a lipase like-activity to allow the protein having an Fc region to be formulated at high concentrations with a polysorbate (e.g., PS20 or PS80) or other fatty acid ester surfactants.

### Surfactants/Polysorbates

In some embodiments, the surfactant in the formulations described herein, the stability of which can be improved using the methods described herein, is a polyester surfactant.

In some embodiments, the surfactant in the formulations described herein, the stability of which can be improved using the methods described herein, is a polysorbate.

A description of illustrative polysorbates that may be used in pharmaceutical compositions is provided in Wucher et al., Journal of Pharmaceutical Sciences Volume 111, Issue 5, May 2022, pp. 1280-1291, which is specifically incorporated by reference herein for examples of polysorbates that may be present in the formulations described herein, as well as their properties and degradation products.

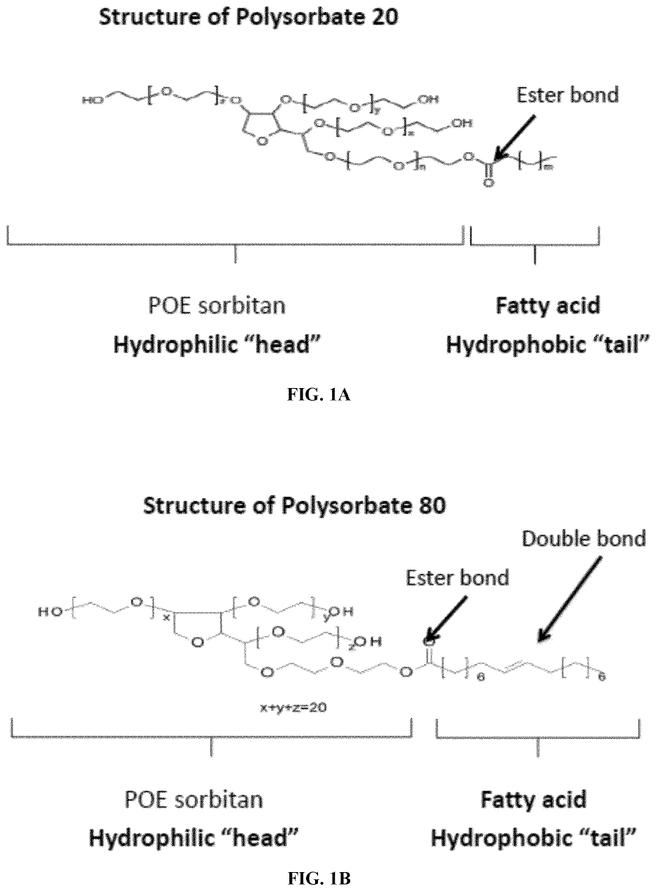

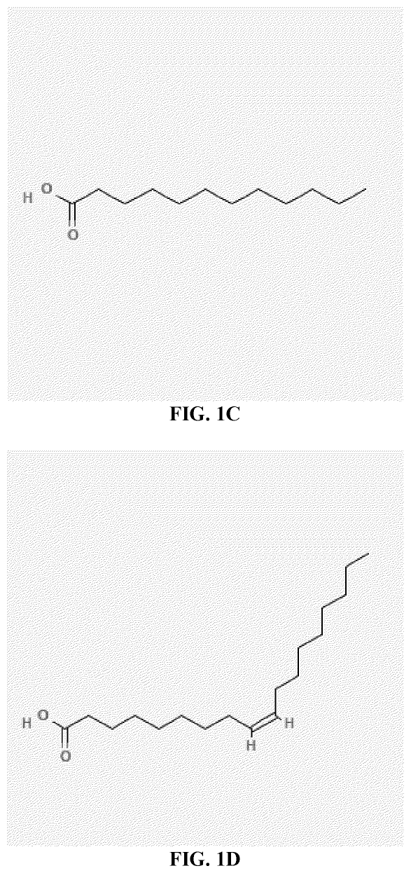

In some embodiments, the polysorbate is PS20. In some embodiments, the polysorbate is PS80. In some embodiment, the polysorbate has a shorter chain of fatty acids (e.g., less or equal to C5, C6, C7, C8, C9, C10 or C11) (see Kerwin, J. of Pharm. Sciences 97: 2924, 2008). In some embodiment, the polysorbate has a longer chain of fatty acids (e.g., more or equal to C12, C13, C14, C15, C16, C17, or C18). In some embodiments, the polysorbate is polyester type ligand. In some embodiments, the polysorbate is a polyamide. In some embodiments, the polysorbate is L-arginine-trehalose. In some embodiments, the polysorbate is L-histidine-trehalose. The structures of Polysorbate 20 and Polysorbate 80 are shown in FIGS. 1A and 1B, respectively. Polysorbate 20 and Polysorbate 80 mainly differ in the esterified hydrophobic tail (see FIGS. 1A and 1B Kerwin, J. of Pharm. Sciences 97: 2924, 2008)). Both Polysorbate 20 and Polysorbate 80 are heterogeneous mixtures of esterified fatty acids with reduced solubility. Generally, Polysorbate 20 will contain about 20-40% of polyesters, about 30-60% of monoesters and some other impurities.

Polysorbate are degraded via oxidation, a chemical reaction, and hydrolysis, an enzymatic reaction. When degradation occurs, polyesters degrade to form monoesters and free fatty acids, and monoesters degrade to form free fatty acids and other residues. Oxidation of polysorbates results in the formation of a mixture of different products, fatty acid esters, fatty acids (about 15% of degradation products), aldehydes, ketones, acids, peroxides and alkanes. Hydrolysis of polysorbates results in the formation of fatty acids and lower order fatty acid esters (see Dwivedi et al., Int. J. of Pharmaceutics 552: 422, 2018).

The key degradation product of Polysorbate 20 is lauric acid in addition to fatty acid esters. The key degradation products of Polysorbate 80 are oleic acid and fatty acid esters (see Dwivedi et al., Int. J. of Pharmaceutics 552: 422, 2018). The structures of lauric acid and oleic acid are shown in FIGS. 1C and 1D, respectively.

The degradation mechanism can be determined using, for example, Free Fatty Acids (FFA) stoichiometry or by incubation with antioxidants (see Tomlinson et al., Mol. Pharm. 12:3805, 2015). With FFA stoichiometry, the polysorbate levels and FFA levels are measured at two time points, and if about 100% of FFAs are recovered, degradation is most likely hydrolytic. If about 15% of FFAs (2-8° C.) are recovered, degradation is likely oxidative. For analysis by incubation with antioxidants, samples are incubated with and without antioxidants and polysorbate content is measured (see Tomlinson et al., Mol. Pharm. 12:3805, 2015). If antioxidants reduce the degradation rate, the degradation has an oxidative component (although, at 40° C. oxidation is inevitable). Enzymatic degradation can be inhibited by lipase inhibitors. Degradation products formed as a result of oxidative degradation can be detected using stir-bar-sorptive extraction coupled to GC-MS (see, e.g., Kishore et al., 2011, Pharmaceutical Research Vol. 28 28, pages 1194-1210). Free fatty acids formed as a result of hydrolytic degradation results can be detected by UPLC (following fluorescent labeling, see, e.g., Tomlinson et al., Mol. Pharmaceutics 2015, 12, 11, 3805-3815) or by LC-MS (see Dwivedi et al., Int. J. of Pharmaceutics 552: 422, 2018). In some embodiments, polysorbate degradation products in the formulations described herein are detected by any methods known in the art or described herein.

The polysorbate degradation may occur as a result of enzymatic hydrolysis due to lipase activity, wherein the lipase(s) are host cell proteins (HCPs) (see Li et al., Antib Ther. 5:42, 2022). In some embodiments, a lipase assay is used to identify lipolytic activity in the formulations described herein. In some embodiments, stability testing, such as polysorbate stability testing described herein, can be used as a surrogate for testing lipase activity.

Polysorbates in a formulation can be protected against oxidation by co-formulating with an antioxidant such as butylated hydroxyanisole (BHA) and/or butylated hydroxytoluene (BHT) (see Schmidt et al., J. of Pharm. Sci. 109:1924, 2020). In some embodiments, the formulations described herein do not comprise an antioxidant. In some embodiments, the formulations described herein do not comprise BHA and/or BHT. For example, Polysorbate 80 can be protected against oxidation by co-formulating it with antioxidants such as BHA and/or BHT. In some embodiments, the formulations described herein comprising Polysorbate 80 do not comprise an antioxidant. In some embodiments, the formulations described herein comprising Polysorbate 80 do not comprise BHA and/or BHT. In some embodiments, the formulations described herein comprising Polysorbate 20 do not comprise an antioxidant. In some embodiments, the formulations described herein comprising Polysorbate 20 do not comprise BHA and/or BHT. In some embodiments, the formulations described herein further comprise an antioxidant.

In some embodiments, the formulations described herein do not comprise a lipase inhibitor or other esterase inhibitors (see Roy et al., J. Pharm. Sci. 119:3313, 2021). In some embodiments, the formulations described herein comprising Polysorbate 20 do not comprise a lipase inhibitor. In some embodiments, the formulations described herein comprising Polysorbate 80 do not comprise a lipase inhibitor. In some embodiments, the formulations described herein further comprise a lipase inhibitor.

The stability of a polysorbate in a formulation may be monitored directly, e.g., by measuring the levels of the polysorbate or by measuring the levels of polysorbate degradation products, such as non-esterified species of PS20 (e.g., Sorbitan or Isosorbide), lauric acid or oleic acid (see Dwivedi et al., Int. J. of Pharmaceutics 552: 422, 2018).

Methods of measuring levels of polysorbates in a formulation are known in the art and described herein and include, for example, fluorescence micelle assays (FMAs), high performance liquid chromatography (HPLC) with Charged Aerosol Detector (CAD) or with evaporative light scattering detector (ELSD), or using liquid chromatography/mass spectrometry (LC/MS) (see Dwivedi et al., Int. J. of Pharmaceutics 552: 422, 2018). Methods of measuring levels of non-esterified species of a polysorbate (e.g., PS20) are known in the art and described herein and include, for example, LCMS. In some embodiments, polysorbate levels in the formulations described herein are detected by any methods known in the art or described herein.

FMA may be used for quantification of PS20 and/or PS80 using HPLC (reaction coil) or a plate reader configurations (see Dwivedi et al., Int. J. of Pharmaceutics 552: 422, 2018). Fluorescence quantum yield of N phenyl 1 naphthylamine (NPN) increases in hydrophobic environment. Fluorescence (emission) intensity increases with micelle concentration, i.e. with polysorbate concentration. In some embodiments, proteins are precipitated with organic solvent (acetonitrile, acetone, etc.) and the organic solvent is removed prior to the FMA measurement. POE sorbitanmonolaureate is only detectable using mixed mode HPLC with ELSD/CAD detection. In HPLC, the eluent is nebulized by inert gas and volatile constituents are evaporated from the droplets. Non-volatile components are detected by light scattering (ELSD) or ionized by positively charged nitrogen gas from a high-voltage platinum corona and quantified by an electrometer (CAD).

### Proteins Comprising an Fc Domain

In some embodiments, the formulations described herein comprise any protein having an Fc region. In some embodiments, the formulations described herein comprise any protein having an Fc region, which can be purified using the methods described herein (e.g., an antibody).

In some embodiments, the protein having an Fc region is an antibody (or an Fc-containing fragment of an antibody). The antibody may be of any of the classes IgG, IgM, IgA, IgE and IgD, which differ from one another by the nature of the heavy chain present in the molecule (see Schroeder et at., J. Allergy Clin. Immunol., 125:S41, 2010). In some embodiments, the antibody is an IgG antibody The antibody may also be of any subclass, e.g., IgG1, IgG2, IgG3, or IgG4. In some embodiments, the antibody is an IgG1 antibody. In some embodiments, the antibody is an IgG4 antibody. In some embodiments, the antibody is a humanized or fully human monoclonal antibody. In some embodiments, the antibody is a human antibody. In some embodiments, the antibody is a mouse antibody. In some embodiments, the antibody is a full-length antibody.

In some embodiments, the antibody is an anti-FcRn antibody. In some embodiments, the anti-FcRn antibody is Batoclimab (also referenced herein as IMVT-1401 and HL161BKN), described, for example, in International Patent Application Publications No. WO 2015/167293, No. WO 2023/235679 and No. WO 2022/005113, each of which is incorporated herein in its entirety for examples of antibodies that may be present in the formations described herein.

In some embodiments, the antibody is a therapeutic antibody. In some embodiments, the antibody is a therapeutic antibody approved by the FDA for the treatment of a disease, or an antibody undergoing clinical studies for the treatment of a disease. In some embodiments, the antibody is selected from Humira® (adalimumab), Remicade® (infliximab), Keytruda® (pembrolizumab), Susvimo® (ranibizumab), Aduhelm® (aducanumab-avwa), Evkeeza® (evinacumab-dgnb), Enspryng® (satralizumab-mwge), PHESGO (pertuzumab, trastuzumab, and hyyaluronidase-zzxf), Vyepti® (eptinezumab-jjmr), Beovu® (brolucizumab-dbll), Herceptin Hylecta® (trastuzumab and hyaluronidase-oysk), Aimovig® (erenumab-aooe), Ajovy® (fremanezumab-vfrm), Emgality® (galcanezumab-gnlm), Ilumya® (tildrakizumab-asmn), Takhzyro® (lanadelumab-flyo), Trogarzo® (ibalizumab-uiyk), Dupixent® (dupilumab), Hemlibra® (emicizumab-kxwh), Kevzara® (sarilumab), Anthim® (obiltoxaximab), Zinbryta® (daclizumab), Cosentyx® (secukinumab), Nucala® (mepolizumab), Praluent® (alirocumab), Repatha® (evolocumab), Benlysta® (belimumab), Actemra® (tocilizumab), Ilaris® (canakinumab), Simponi® (golimumab), Cimzia® (certolizumabpegol), Raptiva (efalizumab), Xolair® (omalizumab), and Synagis® (palivizumab). See Wang et al., 2021, Antibody Therapeutics, Vol. 4, No. 4, pp. 262-273 which incorporated by reference herein in its entirety. Wang et al. is specifically incorporated by reference herein for disclosure of antibodies formulated at high-concentration and their excipients for use in the methods and formulations described herein.

In some embodiment, the protein having an Fc region is an Fc fusion protein. In some embodiments, the Fc fusion protein is a GAS6 Binding Protein-Fc fusion protein, for example, batiraxcept or Enbrel® (etanercept).

### Anti-FcRn Antibodies

In certain aspects, the formulation comprising a protein having an Fc region produced by the methods described herein comprises an anti-FcRn antibody (or an Fc region-containing fragment thereof). In some embodiments, the protein having an Fc region present in the formulations described herein is an anti-FcRn antibody (or an Fc region-containing fragment thereof). In some embodiments, the anti-FcRn antibody is an antibody described in International Patent Publication No. WO/2015/167293 or WO 2023/235679, each of which is incorporated herein by reference in its entirety.

Illustrative CDR sequences of anti-FcRn antibodies are set forth in Table 1

TABLE 1
CDR sequences of heavy chain and light chain variable domains of select human FcRn
antibodies

| | Heavy chain variable domain CDR | | | Light chain variable domain CDR | | |
|---|---|---|---|---|---|---|
| Antibody | CDR1 | CDR2 | CDR3 | CDR1 | CDR2 | CDR3 |
| HL161A | SCVMT | VISGSGGS | TPWWLRS | GGNNIGS | DDSDRP | VRDSSSD |
| | (SEQ ID | TYYADSV | PFFDY | TSVH | S (SEQ ID | HVI (SEQ |
| | NO: 21) | KG (SEQ ID | (SEQ ID | (SEQ ID | NO: 25) | ID NO: 26) |
| | | NO: 22) | NO: 23) | NO: 24) | | |
| HL161B | FSYWV | TIYYSGNT | RAGILTGY | GGNNIGS | DDSDRPS | QVWDSSS |
| (HL161BK) | (SEQ ID | YYNPSLKS | LDS (SEQ | KSVH | (SEQ ID | DHVV |
| (HL161BKN) | NO: 27) | (SEQ ID | ID NO: 29) | (SEQ ID | NO: 31) | (SEQ ID |
| | | NO: 28) | | NO: 30) | | NO: 32) |
| HL161C | GCYMH | RINPNSGG | DYSGWSF | RASQGIS | AASSLOS | QQYDSYP |
| | (SEQ ID | TNYAQKF | DY (SEQ | NYLA | (SEQ ID | PTF (SEQ |
| | NO: 33) | QG (SEQ ID | ID NO: 35) | (SEQ ID | NO: 37) | ID NO: 38) |
| | | NO: 34) | | NO: 36) | | |
| HL161D | SYYWG | NIYYSGST | QLSYNWN | GGNNIGS | RDSNRPS | QVWDSST |
| | (SEQ ID | YYNPSLM | DRLFDY | KNVH | (SEQ ID | VV (SEQ |
| | NO: 39) | S (SEQ ID | (SEQ ID | (SEQ ID | NO: 43) | ID NO: 44) |
| | | NO: 40) | NO: 41) | NO: 42) | | |
| HL161ANS | SSVMT | VISGSGGS | TPWWLRS | GGSNIGS | DDSDRPS | VRDSSSD |
| | (SEQ ID | TYYADSV | PFFDY | TSVH | (SEQ ID | HVI (SEQ |
| | NO: 49) | KG (SEQ ID | (SEQ ID | (SEQ ID | NO: 25) | ID NO: 26) |
| | | NO: 22) | NO: 23) | NO 50) | | |

In some embodiments, the antibody comprises three heavy chain CDR amino acid sequences of SEQ ID No: 27 (HCDR1), SEQ ID No: 28 (HCDR2), SEQ ID No: 29 (HCDR3); and three light chain CDR amino acid sequences of SEQ ID No: 30 (LCDR1), SEQ ID No: 31 (LCDR2), SEQ ID No: 32 (LCDR3). In some embodiments, the antibody comprises three heavy chain CDR amino acid sequences of SEQ ID No: 49 (HCDR1), SEQ ID No: 22 (HCDR2), SEQ ID No: 23 (HCDR3); and three light chain CDR amino acid sequences of SEQ ID No: 50 (LCDR1), SEQ ID No: 25 (LCDR2), SEQ ID No: 26 (LCDR3).

Illustrative heavy and light chain variable regions of anti-FcRn antibodies are set forth in Table 2.

TABLE 2
Amino acid sequences of heavy chain and light chain variable domains of
select human FcRn antibodies

| | Heavy chain variable | Light chain variable |
|---|---|---|
| Antibody | domain amino acid Sequence | domain amino acid sequence |
| HL161A | EVQLLESGGG LVQPGGSLRL | SYVLTQPPSV SVAPGQTARI |
| | SCAASEFTFG SCVMTWVRQA | TCGGNNIGST SVHWYQQKPG |
| | PGKGLEWVSV ISGSGGSTYY | QAPVLVVHDD SDRPSGIPER |
| | ADSVKGRFTI SRDNSKNTLY | FSGSNSGNTA TLTISRVEAG |
| | LQMNSLRAED TAVYYCAKTP | DEADYYCQVR DSSSDHVIFG |
| | WWLRSPFFDY WGQGTLVTVSS | GGTKLTVLGQ PKAAPSVTL (SEQ |
| | (SEQ ID NO: 2) | ID NO: 12) |
| HL161B | QLLLQESGPG LVKPSETLSL | SYVLTQSPSV SVAPGQTARI |
| | TCTVSGGSLS SSFSYWVWIR | TCGGNNIGSK SVHWYQQKPG |
| | QPPGKGLEWI GTIYYSGNTY | QAPVLVVYDD SDRPSGIPER |
| | YNPSLKSRLT ISVDTSKNHF | FSASNSGNTA TLTISRVEAG |
| | SLNLSSVTAA DTAVYYCARR | DEADYYCQVW DSSSDHVVFG |
| | AGILTGYLDS WGQGTLVTVSS | GGTKLTVLGQ PKAAPSVTL (SEQ |
| | (SEQ ID NO: 4) | ID NO: 14) |
| HL161BK | QLLLQESGPG LVKPSETLSL | SYVLTQSPSV SVAPGQTARI |
| (HL161BKN) | TCTVSGGSLS SSFSYWVWIR | TCGGNNIGSK SVHWYQQKPG |
| | QPPGKGLEWI GTIYYSGNTY | QAPVLVVYDD SDRPSGIPER |
| | YNPSLKSRLT ISVDTSKNHF | FSASNSGNTA TLTISRVEAG |
| | SLKLSSVTAA DTAVYYCARR | DEADYYCQVW DSSSDHVVFG |
| | AGILTGYLDS WGQGTLVTVSS | GGTKLTVLGQ PKAAPSVTL (SEQ |
| | (SEQ ID NO: 6) | ID NO: 16) |
| HL161C | QVQLVQSGAE VKKPGASVKV | DIQMTQSPSS LSASVGDRVT |
| | SCKASGYTFT GCYMHWVRQA | ITCRASQGIS NYLAWFQQKP |
| | PGQGLEWMGR INPNSGGTNY | GKAPKSLIYA ASSLQSGVPS |
| | AQKFQGRVTM TRDTSISTAY | KFSGSGSGTD FTLTISSLQS |
| | MDLSRLRSDD TAVYYCARDY | EDFATYYCQQ YDSYPPTFGG |
| | SGWSFDYWGQ GTLVTVSS | GTKVEIKRTV AAPSVFI (SEQ ID |
| | (SEQ ID NO: 8) | NO: 18) |
| HL161D | QLQLQESGPG LVKPSETLSL | SYELTQPLSV SVALGQTARI |
| | TCTVSGGSIS SSSYYWGWIR | TCGGNNIGSK NVHWYQQKPG |
| | QPPGKGLEWI GNIYYSGSTY | QAPVLVIYRD SNRPSGIPER |
| | YNPSLMSRVT ISVDTSKNQF | FSGSNSGNTA TLTISRAQAG |
| | SLKLSSVTAA DTAVYYCARQ | DEADYYCQVW DSSTVVFGGG |
| | LSYNWNDRLF DYWGQGTLVT VSS | TKLTVLGQPK AAPSVTL |
| | (SEQ ID NO: 10) | (SEQ ID NO: 20) |
| HL161ANS | EVQLLESGGG LVQPGGSLRL | SYVLTQPPSV SVAPGQTARI |
| | SCAASEFTFG SSVMTWVRQA | TCGGSNIGST SVHWYQQKPG |
| | PGKGLEWVSV ISGSGGSTYY | QAPVLVVHDD SDRPSGIPER |
| | ADSVKGRFTI SRDNSKNTLY | FSGSNSGNTA TLTISRVEAG |
| | LQMNSLRAED TAVYYCAKTP | DEADYYCQVR DSSSDHVIFG |
| | WWLRSPFFDY WGQGTLVTVSS | GGTKLTVLGQ PKAAPSVTL (SEQ |
| | (SEQ ID NO: 51) | ID NO: 52) |

In some embodiments, the antibody comprises a heavy chain variable region comprising an amino acid sequence that is at least 90%, at least 95%, at least 98%, or least 99% identical to the sequence set forth in SEQ ID No: 6; and a light chain variable region comprising an amino acid sequence that is at least 90%, at least 95%, at least 98%, or least 99% identical to the sequence set forth in SEQ ID No: 16. In some embodiments, the antibody comprises a heavy chain amino acid comprising an amino acid sequence that is at least 90%, at least 95%, at least 98%, or least 99% identical to the sequence set forth in SEQ ID No: 46; and a light chain amino acid comprising an amino acid sequence that is at least 90%, at least 95%, at least 98%, or least 99% identical to the sequence set forth in SEQ ID No: 48. In some embodiments, the antibody comprises a heavy chain amino acid comprising an amino acid sequence that is at least 90%, at least 95%, at least 98%, or least 99% identical to the sequence set forth in SEQ ID No: 51; and a light chain amino acid comprising an amino acid sequence that is at least 90%, at least 95%, at least 98%, or least 99% identical to the sequence set forth in SEQ ID No: 52.

In some embodiments, the antibody comprises a heavy chain variable region amino acid sequence of SEQ ID No: 6; and a light chain variable region amino acid sequence of SEQ ID No: 16. In some embodiments, the antibody comprises a heavy chain amino acid sequence of SEQ ID No: 46; and a light chain amino acid sequence of SEQ ID No: 48. In some embodiments, the antibody comprises a heavy chain amino acid sequence of SEQ ID No: 51; and a light chain amino acid sequence of SEQ ID No: 52.

Illustrative heavy chains and light chains of the anti-FcRn antibodies are set forth in Table 3.

TABLE 3
Amino acid sequences of full-length heavy chain and light chain of select human FcRn
antibodies

| Antibody | | |
|---|---|---|
| name | Heavy chain amino acid sequence | Light chain amino acid sequence |
| HL161BKN | QLLLQESGPG LVKPSETLSL TCTVSGGSLS | SLTPEQWKSH RSYSCQVTHE |
| | SSFSYWVWIR QPPGKGLEWI GTIYYSGNTY | SYVLTQSPSV SVAPGQTARI |
| | YNPSLKSRLT ISVDTSKNHF SLKLSSVTAA | TCGGNNIGSK SVHWYQQKPG |
| | DTAVYYCARR AGILTGYLDS WGQGTLVTVS | QAPVLVVYDD SDRPSGIPER |
| | SASTKGPSVF PLAPSSKSTS GGTAALGCLV | FSASNSGNTA TLTISRVEAG |
| | KDYFPEPVTV SWNSGALTSG VHTFPAVLQS | DEADYYCQVW DSSSDHVVFG |
| | SGLYSLSSVV TVPSSSLGTQ | GGTKLTVLGQ PKAAPSVTLF |
| | TYICNVNHKP SNTKVDKRVE PKSCDKTHTC | PPSSEELQAN KATLVCLISD |
| | PPCPAPEAAG GPSVFLFPPK PKDTLMISRT | FYPGAVTVAW KADSSPVKAG |
| | PEVTCVVVDV SHEDPEVKFN WYVDGVEVHN | VETTTPSKQS NNKYAASSYL |
| | AKTKPREEQY NSTYRVVSVL TVLHQDWLNG | GSTVEKTVAP TECS (SEQ ID |
| | KEYKCKVSNK ALPAPIEKTI SKAKGQPREP | NO: 48) |
| | QVYTLPPSRE EMTKNQVSLT CLVKGFYPSD | |
| | IAVEWESNGQ PENNYKTTPP VLDSDGSFFL | |
| | YSKLTVDKSR WQQGNVFSCS | |
| | VMHEALHNHY TQKSLSLSPG (SEQ ID NO: 46) | |
| HL161ANS | EVQLLESGGG LVQPGGSLRL SCAASEFTFG | FSGSNSGNTA TLTISRVEAG |
| IgG1 | SSVMTWVRQA PGKGLEWVSV ISGSGGSTYY | SLTPEQWKSH RSYSCQVTHE |
| | ADSVKGRFTI SRDNSKNTLY LQMNSLRAED | SYVLTQPPSV SVAPGQTARI |
| | TAVYYCAKTP WWLRSPFFDY WGQGTLVTVS | TCGGSNIGST SVHWYQQKPG |
| | SASTKGPSVF PLAPSSKSTS GGTAALGCLV | QAPVLVVHDD SDRPSGIPER |
| | KDYFPEPVTV SWNSGALTSG VHTFPAVLQS | DEADYYCQVR DSSSDHVIFG |
| | SGLYSLSSVV TVPSSSLGTQ | GGTKLTVLGQ PKAAPSVTLF |
| | TYICNVNHKP SNTKVDKRVE PKSCDKTHTC | PPSSEELQAN KATLVCLISD |
| | PPCPAPEAAG GPSVFLFPPK PKDTLMISRT | FYPGAVTVAW KADSSPVKAG |
| | PEVTCVVVDV SHEDPEVKFN WYVDGVEVHN | VETTTPSKQS NNKYAASSYL |
| | AKTKPREEQY NSTYRVVSVL TVLHQDWLNG | GSTVEKTVAP TECS |
| | KEYKCKVSNK ALPAPIEKTI SKAKGQPREP | (SEQ ID NO: 54) |
| | QVYTLPPSRE EMTKNQVSLT CLVKGFYPSD | |
| | IAVEWESNGQ PENNYKTTPP | |
| | VLDSDGSFFL YSKLTVDKSR WQQGNVFSCS | |
| | VMHEALHNHY TQKSLSLSPG (SEQ ID NO: 53) | |
| HL161ANS | EVQLLESGGG LVQPGGSLRL SCAASEFTFG | SYVLTQPPSV SVAPGQTARI |
| IgG4 | SSVMTWVRQA PGKGLEWVSV ISGSGGSTYY | TCGGSNIGST SVHWYQQKPG |
| | ADSVKGRFTI SRDNSKNTLY LQMNSLRAED | QAPVLVVHDD SDRPSGIPER |
| | TAVYYCAKTP WWLRSPFFDY WGQGTLVTVS | FSGSNSGNTA TLTISRVEAG |
| | SASTKGPSVF PLAPCSRSTS ESTAALGCLV | DEADYYCQVR DSSSDHVIFG |
| | KDYFPEPVTV SWNSGALTSG VHTFPAVLQS | GGTKLTVLGQ PKAAPSVTLF |
| | SGLYSLSSVV TVPSSSLGTK TYTCNVDHKP | PPSSEELQAN KATLVCLISD |
| | SNTKVDKRVE SKYGPPCPPC PAPEFLGGPS | FYPGAVTVAW KADSSPVKAG |
| | VFLFPPKPKD TLMISRTPEV TCVVVDVSQE | VETTTPSKQS NNKYAASSYL |
| | DPEVQFNWYV DGVEVHNAKT KPREEQFNST | SLTPEQWKSH RSYSCQVTHE |
| | YRVVSVLTVL HQDWLNGKEY KCKVSNKGLP | GSTVEKTVAP TECS (SEQ ID |
| | SSIEKTISKA KGQPREPQVY TLPPSQEEMT | NO: 56) |
| | KNQVSLTCLV KGFYPSDIAV EWESNGQPEN | |
| | NYKTTPPVLD SDGSFFLYSR LTVDKSRWQE | |
| | GNVFSCSVMH EALHNHYTQK SLSLSLG (SEQ | |
| | ID NO: 55) | |

In some embodiments, the antibody comprises a heavy chain comprising an amino acid sequence that is at least 90%, at least 95%, at least 98%, or least 99% identical to the sequence set forth in SEQ ID No: 46, 53, or 55; and a light chain comprising an amino acid sequence that is at least 90%, at least 95%, at least 98%, or least 99% identical to the sequence set forth in SEQ ID No: 48, 54, or 56. In some embodiments, the antibody comprises a heavy chain comprising the sequence set forth in SEQ ID No: 46, 53, or 55; and a light chain comprising the sequence set forth in SEQ ID No: 48, 54, or 56.

In some embodiments, the anti-FcRn antibody comprises:
a CDR1 comprising an amino acid sequence that is at least 90% identical to one or more amino acid sequences selected from the group consisting of SEQ ID Nos: 21, 24, 27, 30, 33, 36, 39, 42, 49, and 50;
a CDR2 comprising an amino acid sequence that is at least 90% identical to one or more amino acid sequences selected from the group consisting of SEQ ID Nos: 22, 25, 28, 31, 34, 37, 40, and 43; and
a CDR3 comprising an amino acid sequence that is at least 90% identical to one or more amino acid sequences selected from the group consisting of SEQ ID Nos: 23, 26, 29, 32, 35, 38, 41, and 44.

In some embodiments, the anti-FcRn antibody comprises:
a CDR1 comprising an amino acid sequence that is at least 91%, at least 92%, at least 93%, at least 94%, at least 95%, at least 96%, at least 97%, at least 98%, or at least 99% identical to one or more amino acid sequences selected from the group consisting of SEQ ID Nos: 21, 24, 27, 30, 33, 36, 39, 42, 49 and 50;
a CDR2 comprising an amino acid sequence that is at least 91%, at least 92%, at least 93%, at least 94%, at least 95%, at least 96%, at least 97%, at least 98%, or at least 99% identical to one or more amino acid sequences selected from the group consisting of SEQ ID Nos: 22, 25, 28, 31, 34, 37, 40, and 43; and
a CDR3 comprising an amino acid sequence that is at least 91%, at least 92%, at least 93%, at least 94%, at least 95%, at least 96%, at least 97%, at least 98%, or at least 99% identical to one or more amino acid sequences selected from the group consisting of SEQ ID Nos: 23, 26, 29, 32, 35, 38, 41, and 44.

In some embodiments, the anti-FcRn antibody is an antibody having (i) the CDRs of HL161BKN, (ii) heavy and/or light chain variable domains of HL161BKN, or (iii) heavy and/or light chains of HL161BKN. In some embodiments, the anti-FcRn antibody is an antibody having (i) the CDRs of HL161B, (ii) heavy and/or light chain variable domains of HL161B, or (iii) heavy and/or light chains of HL161B. In some embodiments, the anti-FcRn antibody is an antibody having (i) the CDRs of HL161A, (ii) heavy and/or light chain variable domains of HL161A, or (iii) heavy and/or light chains of HL161A. In some embodiments, the anti-FcRn antibody is an antibody having (i) the CDRs of HL161C, (ii) heavy and/or light chain variable domains of HL161C, or (iii) heavy and/or light chains of HL161C. In some embodiments, the anti-FcRn antibody is an antibody having (i) the CDRs of HL161D, (ii) heavy and/or light chain variable domains of HL161D, or (iii) heavy and/or light chains of HL161D. In some embodiments, the anti-FcRn antibody is an antibody having (i) the CDRs of HL161ANS, (ii) heavy and/or light chain variable domains of HL161ANS, or (iii) heavy and/or light chains of HL161ANS. The respective sequences of these antibodies are provided herein.

In some embodiments, the anti-FcRn antibody may comprise one or more amino acid deletions, additions, or substitutions in the amino acid sequences described herein.

In some embodiments, the anti-FcRn antibody may comprise amino acid sequences identical to or having homology with the amino acid sequences described herein. The term “identity” or “homology” refers to a relationship between the sequences of two or more polypeptides, as determined by comparing the sequences. The term “identity” also means the degree of sequence relatedness between the polypeptides, as determined by the number of matches between strings of two or more amino acid residues. The percent “identity” between the two sequences is a function of the number of identical positions shared by the sequences (i.e., percent identity equals number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Additionally, or alternatively, the amino acid sequences disclosed herein can further be used as a “query sequence” to perform a search against public databases to, for example, identify related sequences. For example, such searches can be performed using the BLAST program of Altschul et al. (J. Mol. Biol. 215:403-10, 1990).

Two sequences are “substantially identical” if two sequences have a specified percentage of amino acid residues that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 10 amino acids in length, or over a region that is about 20, 50, 200 or more amino acids in length. In some embodiments, the anti-FcRn antibodies described herein comprise at least one amino acid sequence that is at least 90% identical to a sequence selected from the group consisting of SEQ ID Nos: 2, 4, 6, 8, 10, 12, 14, 16, 18, and 20-48. In some embodiments, the anti-FcRn antibodies described herein comprise at least one amino acid sequence that is at least 91%, at least 92%, at least 93%, at least 94%, at least 95%, at least 96%, at least 97%, at least 98%, or at least 99% identical to a sequence selected from the group consisting of SEQ ID Nos: 2, 4, 6, 8, 10, 12, 14, 16, 18, and 20-48.

In some embodiments, the antibody comprises a heavy chain variable region comprising:
a CDR1 comprising an amino acid sequence of SEQ ID No: 21, CDR2 comprising an amino acid sequence of SEQ ID No: 22, and CDR3 comprising an amino acid sequence of SEQ ID No: 23;
a CDR1 comprising an amino acid sequence of SEQ ID No: 27, CDR2 comprising an amino acid sequence of SEQ ID No: 28, and CDR3 comprising an amino acid sequence of SEQ ID No: 29;
a CDR1 comprising an amino acid sequence of SEQ ID No: 33, CDR2 comprising an amino acid sequence of SEQ ID No: 34, and CDR3 comprising an amino acid sequence of SEQ ID No: 35;
a CDR1 comprising an amino acid sequence of SEQ ID No: 39, CDR2 comprising an amino acid sequence of SEQ ID No: 40, and CDR3 comprising an amino acid sequence of SEQ ID No: 41; or
a CDR1 comprising an amino acid sequence of SEQ ID No: 49, CDR2 comprising an amino acid sequence of SEQ ID No: 22, and CDR3 comprising an amino acid sequence of SEQ ID No: 23.

In some embodiments, the antibody comprises a light chain variable region comprising:
a CDR1 comprising an amino acid sequence of SEQ ID No: 24, CDR2 comprising an amino acid sequence of SEQ ID No: 25, and CDR3 comprising an amino acid sequence of SEQ ID No: 26;
a CDR1 comprising an amino acid sequence of SEQ ID No: 30, CDR2 comprising an amino acid sequence of SEQ ID No: 31, and CDR3 comprising an amino acid sequence of SEQ ID No: 32;
a CDR1 comprising an amino acid sequence of SEQ ID No: 36, CDR2 comprising an amino acid sequence of SEQ ID No: 37, and CDR3 comprising an amino acid sequence of SEQ ID No: 38;
a CDR1 comprising an amino acid sequence of SEQ ID No: 42, CDR2 comprising an amino acid sequence of SEQ ID No: 43, and CDR3 comprising an amino acid sequence of SEQ ID No: 44 or
a CDR1 comprising an amino acid sequence of SEQ ID No: 50, CDR2 comprising an amino acid sequence of SEQ ID No: 25, and CDR3 comprising an amino acid sequence of SEQ ID No: 26.

In some embodiments, the antibody comprises one or more heavy chain variable regions and one or more light chain variable regions selected from the group consisting of:
a heavy chain variable region comprising CDR1 comprising an amino acid sequence of SEQ ID No: 21 (HCDR1), CDR2 comprising an amino acid sequence of SEQ ID No: 22 (HCDR2), and CDR3 comprising an amino acid sequence of SEQ ID No: 23 (HCDR3); and a light chain variable region comprising CDR1 comprising an amino acid sequence of SEQ ID No: 24 (LCDR1), CDR2 comprising an amino acid sequence of SEQ ID No: 25 (LCDR2), and CDR3 comprising an amino acid sequence of SEQ ID No: 26 (LCDR3);
a heavy chain variable region comprising CDR1 comprising an amino acid sequence of SEQ ID No: 27 (HCDR1), CDR2 comprising an amino acid sequence of SEQ ID No: 28 (HCDR2), and CDR3 comprising an amino acid sequence of SEQ ID No: 29 (HCDR3); and a light chain variable region comprising CDR1 comprising an amino acid sequence of SEQ ID No: 30 (LCDR1), CDR2 comprising an amino acid sequence of SEQ ID No: 31 (LCDR2), and CDR3 comprising an amino acid sequence of SEQ ID No: 32 (LCDR3);
a heavy chain variable region comprising CDR1 comprising an amino acid sequence of SEQ ID No: 33 (HCDR1), CDR2 comprising an amino acid sequence of SEQ ID No: 34 (HCDR2), and CDR3 comprising an amino acid sequence of SEQ ID No: 35 (HCDR3); and a light chain variable region comprising CDR1 comprising an amino acid sequence of SEQ ID No: 36 (LCDR1), CDR2 comprising an amino acid sequence of SEQ ID No: 37 (LCDR2), and CDR3 comprising an amino acid sequence of SEQ ID No: 38 (LCDR3);
a heavy chain variable region comprising CDR1 comprising an amino acid sequence of SEQ ID No: 39 (HCDR1), CDR2 comprising an amino acid sequence of SEQ ID No: 40 (HCDR2), and CDR3 comprising an amino acid sequence of SEQ ID No: 41 (HCDR3); and a light chain variable region comprising CDR1 comprising an amino acid sequence of SEQ ID No: 42 (LCDR1), CDR2 comprising an amino acid sequence of SEQ ID No: 43 (LCDR2), and CDR3 comprising an amino acid sequence of SEQ ID No: 44 (LCDR3); and
a heavy chain variable region comprising CDR1 comprising an amino acid sequence of SEQ ID No: 49 (HCDR1), CDR2 comprising an amino acid sequence of SEQ ID No: 22 (HCDR2), and CDR3 comprising an amino acid sequence of SEQ ID No: 23 (HCDR3); and a light chain variable region comprising CDR1 comprising an amino acid sequence of SEQ ID No: 50 (LCDR1), CDR2 comprising an amino acid sequence of SEQ ID No: 25 (LCDR2), and CDR3 comprising an amino acid sequence of SEQ ID No: 26 (LCDR3).

In some embodiments, the antibody comprises one or more heavy chain variable regions and/or one or more light chain variable regions comprising one or more amino acid sequences selected from the group consisting of amino acid sequences of SEQ ID Nos: 2, 4, 6, 8, 10, 12, 14, 16, 18, 20, 51, and 52.

In some embodiments, the antibody comprises heavy chain variable region comprising an amino acid sequence of SEQ ID Nos: 2, 4, 6, 8, 10, or 51 and/or light chain variable region comprising an amino acid sequence of SEQ ID Nos: 12, 14, 16, 18, 20, or 52.

In some embodiments, the antibody comprises one or more heavy chain variable regions and one or more light chain variable regions selected from the group consisting of:
a heavy chain variable region comprising an amino acid sequence of SEQ ID No: 2 and a light chain variable region comprising an amino acid sequence of SEQ ID No: 12;
a heavy chain variable region comprising an amino acid sequence of SEQ ID No: 4 and a light chain variable region comprising an amino acid sequence of SEQ ID No: 14;
a heavy chain variable region comprising an amino acid sequence of SEQ ID No: 6 and a light chain variable region comprising an amino acid sequence of SEQ ID No: 16;
a heavy chain variable region comprising an amino acid sequence of SEQ ID No: 8 and a light chain variable region comprising an amino acid sequence of SEQ ID No: 18;
a heavy chain variable region comprising an amino acid sequence of SEQ ID No: 10 and a light chain variable region comprising an amino acid sequence of SEQ ID No: 20; and
a heavy chain variable region comprising an amino acid sequence of SEQ ID No: 51 and a light chain variable region comprising an amino acid sequence of SEQ ID No: 52.

In some embodiments, the antibody is a binding-domain immunoglobulin fusion protein, camelid antibody, VHH containing antibody, IgD antibody, IgE antibody, IgM antibody, IgG1 antibody, IgG2 antibody, IgG3 antibody, or IgG4 antibody. In some embodiments, the antibody comprises derivatives in the antibody constant regions. In some embodiments, the antibody is a synthetic antibody based on protein scaffolds that have the ability to bind to FcRn.

In some embodiments, the anti-FcRn antibody comprises a conservative substitution of an amino acid residue in the variable region and/or constant region. As used herein, the term “conservative substitution” refers to a substitution with another amino acid residue having properties similar to those of the original amino acid residue. For example, lysine, arginine and histidine have similar properties in that they have a basic side-chain, and aspartic acid and glutamic acid have similar properties in that they have an acidic side chain. In addition, glycine, asparagine, glutamine, serine, threonine, tyrosine, cysteine and tryptophan have similar properties in that they have an uncharged polar side-chain, and alanine, valine, leucine, threonine, isoleucine, proline, phenylalanine and methionine have similar properties in that they have a non-polar side-chain. Also, tyrosine, phenylalanine, tryptophan and histidine have similar properties in that they have an aromatic side-chain. Thus, it will be obvious to those skilled in the art that, even when substitution of amino acid residues in groups showing similar properties as described above occurs, it will likely show no significant change in the properties of the antibody.

Protein Formulations with Improved Polysorbate Stability

In one aspect, provided herein are formulations comprising a protein having an Fc region and a pharmaceutically acceptable carrier, wherein the pharmaceutically acceptable carrier comprises a surfactant, wherein the formulation has an improved stability of the surfactant.

In one aspect, provided herein are formulations comprising a protein having an Fc region and a pharmaceutically acceptable carrier, wherein the pharmaceutically acceptable carrier comprises one or more excipients comprising a polysorbate, wherein the formulation has an improved stability of the polysorbate.

In some embodiments, the formulations provided herein are pharmaceutical formulations. In some embodiments, the formulations are for use in biomedical applications. In some embodiments, the formulations are for use in diagnostic applications. In some embodiments, the formulations are for use in therapeutic applications, such as for administration to a patient (e.g., a mammal, e.g., human) for treating and/or preventing any condition or disease. The patient can be any mammal, for example, a companion animal (e.g., a dog or cat) or a farm animal (e.g., a horse, a cow or a pig). In some embodiments, the patient is a human.

In some embodiments, the formulation comprises a protein having an Fc region at a high concentration, for example, a concentration of at least or more than 50 g/L, 60 g/L, 70 g/L, 75 g/L, 80 g/L, 85 g/L, 90 g/L, 95 g/L, 100 g/L, 105 g/L, 110 g/L, 115 g/L, 120 g/L, 125 g/L, 130 g/L, 135 g/L, 140 g/L, 145 g/L, 150 g/L, 155 g/L, 160 g/L, 165 g/L, 170 g/L, 175 g/L, 180 g/L, 185 g/L, 190 g/L, 195 g/L, 200 g/L, 210 g/L, 220 g/L, 225 g/L, or 250 g/L. In some embodiments, the formulation comprises a protein having an Fc region at a concentration of about 100 g/L, about 110 g/L, about 120 g/L, about 130 g/L, about 140 g/L, about 150 g/L, about 160 g/L, about 170 g/L, about 180 g/L, about 190 g/L, or about 200 g/L. In some embodiments, the formulation comprises a protein having an Fc region at a high concentration, for example, a concentration of at least or more than about 100 g/L. In some embodiments, the formulation comprises a protein having an Fc region at a high concentration, for example, a concentration of at least or more than about 125 g/L. In some embodiments, the formulation comprises a protein having an Fc region at a high concentration, for example, a concentration of at least or more than about 150 g/L. In some embodiments, the formulation comprises a protein having an Fc region at a high concentration, for example, a concentration of at least or more than about 170 g/L or 175 g/L. In some embodiments, the formulation comprises a protein having an Fc region at a high concentration, for example, a concentration of at least or more than about 200 g/L. In some embodiments, the formulation comprises a protein having an Fc region at a high concentration, for example, a concentration of about 100-110 g/L, about 110-120 g/L, about 120-130 g/L, about 130-140 g/L, about 140-150 g/L, about 150-160 g/L, about 160-170 g/L, about 170-180 g/L, about 180-190 g/L, or about 190-200 g/L. In some embodiments, the formulation comprises a protein having an Fc region at a concentration of about 100 g/L, about 110 g/L, about 120 g/L, about 130 g/L, about 140 g/L, about 150 g/L, about 160 g/L, about 170 g/L, about 180 g/L, about 190 g/L, or about 200 g/L.

In some embodiments, the formulation is a formulation comprising a protein having an Fc region and further comprising a polysorbate (e.g., at any concentration, for example at a concentration from 0.001% to 0.5%, e.g., about 0.005%, 0.01%, 0.02%, 0.03%, 0.04%, 0.05%, or 0.1%, or any concentration in between these values).

In some embodiments, the formulation is a formulation comprising a protein having an Fc region and further comprising Polysorbate 20 (e.g., at any concentration, for example at a concentration from 0.001% to 0.5%, e.g., about 0.005%, 0.01%, 0.02%, 0.03%, 0.04%, 0.05%, or 0.1%, or any concentration in between these values).

In some embodiments, the formulation is a formulation comprising a protein having an Fc region and further comprising Polysorbate 80 (e.g., at any concentration, for example at a concentration from 0.001% to 0.5%, e.g., about 0.005%, 0.01%, 0.02%, 0.03%, 0.04%, 0.05%, or 0.1%, or any concentration in between these values).

In some embodiments, the formulation is a formulation comprising a protein having an Fc region and a polysorbate and further comprising at least one additive selected from mannitol, sorbitol, arginine, histidine, glycine and salts thereof, and/or a buffer system selected from citrate or histidine. In some embodiments, the formulation does not comprise arginine. In some embodiments, the additive is arginine or a salt thereof, and the buffer system is histidine. In some embodiments, the formulation comprises 50 to 250 mM L-arginine or a hydrochloride salt thereof. In some embodiments, the formulation comprises 50 to 250 mM L-histidine buffer. In some embodiments, the formulation comprises 0.01 to 0.05% polysorbate. In some embodiments, the formulation comprises 50 to 250 mM L-arginine or a hydrochloride salt thereof, 50 to 250 mM L-histidine buffer, and 0.01 to 0.05% of a polysorbate. In some embodiments, the formulation is a formulation comprising a protein having an Fc region and a polysorbate and further comprising L-Histidine/Histidine HCl, and L-Arginine HCl. In some embodiments, the protein having an Fc region is at a high concentration in the formulation, e.g., at a concentration equal to or higher than 50 g/L, 75 g/L, 100 g/L, 150 g/L or 170 g/L. In some embodiments, the formulation has a pH of 4.0 to 8.0. In some embodiments, the formulation has a pH of 4.0 to 7.0.

The formulation may be suitable for administration via any desired route, including, for example, intravenous injection, subcutaneous injection, intramuscular injection, intraperitoneal injection, topical administration, intranasal administration, intrapulmonary administration, or intrarectal administration. In some embodiments, the formulation is a pharmaceutical formulation suitable for an injection (e.g., a subcutaneous injection) to a subject (e.g., a mammal, e.g., a human). In some embodiments, the formulation has a viscosity of 20 cP or less. In some embodiments, the formulation has an osmolality of 250 mOs/kg to 500 mOs/kg.

In some embodiments, the formulation is a formulation comprising about or more than 170 mg/mL of an anti-FcRn antibody. In some embodiments, the formulation is a formulation comprising an anti-FcRn antibody and further comprising one or more excipients comprising a polysorbate (e.g., Polysorbate 20 at a concentration from 0.01% to 0.2%, 0.01% to 0.05%, e.g., 0.02%). In some embodiments, the formulation is a formulation comprising an anti-FcRn antibody and a polysorbate and further comprising at least one additive selected from mannitol, sorbitol, arginine, histidine, glycine and salts thereof, and/or a buffer system selected from citrate or histidine. In some embodiments, the additive is arginine or a salt thereof, and the buffer system is histidine. In some embodiments, the formulation comprises 50 to 250 mM L-arginine or a hydrochloride salt thereof. In some embodiments, the formulation comprises 50 to 250 mM L-histidine buffer. In some embodiments, the formulation comprises 0.01 to 0.05% polysorbate. In some embodiments, the formulation comprises an anti-FcRn antibody, 50 to 250 mM L-arginine or a hydrochloride salt thereof, 50 to 250 mM L-histidine buffer, and 0.01 to 0.05% of a polysorbate. In some embodiments, the formulation is a formulation comprising an anti-FcRn antibody and further comprising Polysorbate 20 (e.g., Polysorbate 20 at a concentration from 0.01% to 0.2%, 0.01% to 0.05%, e.g., 0.02%), L-Histidine/Histidine HCl, and L-Arginine HCl. In some embodiments, the anti-FcRn antibody is at a high concentration in the formulation, e.g., at a concentration equal to or higher than 50 g/L, 75 g/L, 100 g/L, 150 g/L or 170 g/L. In some embodiments, the formulation with the anti-FcRn antibody has a pH of 4.0 to 8.0. In some embodiments, the formulation with the anti-FcRn antibody has a pH of 4.0 to 7.0.

In some embodiments, the formulation is a formulation comprising about 170 mg/mL anti-FcRn antibody in 100 mM L-Histidine/Histidine HCl, 100 mM L-Arginine HCl and 0.02% Polysorbate 20 in water at pH 6.

In some embodiments, the formulation, such as a formulation of an anti-FcRn antibody, is for the treatment of an autoimmune disease selected from the group consisting of myasthenia gravis, thyroid eye disease, warm autoimmune hemolytic anemia, neuromyelitis optica, immune thrombocytopenic purpura, pemphigus vulgaris, chronic inflammatory demyelinating polyneuropathy, lupus nephritis, and membranous nephropathy.

The formulation may comprise a protein having an Fc region that has been purified from cell culture (e.g., mammalian host cell culture). In some embodiments, the protein having an Fc region has been purified from cell culture using an Fc-binding affinity resin chromatography column. In some embodiments, the protein having an Fc region has been purified from cell culture using Protein A affinity chromatography. In some embodiments, the protein having an Fc region has been purified from cell culture using Protein G affinity chromatography. In some embodiments, the Fc-binding affinity resin chromatography column comprises a genetically engineered Fc binding protein. In some embodiments, the protein having an Fc region has been purified from cell culture using any method of Fc-binding affinity resin chromatography described herein or known in the art.

In some aspects, the concentration of polysorbate in the formulation remains substantially unchanged for a period of time of at least one day, for example, 1 week, 2 weeks, 4 weeks, 8 weeks, 4 months, 8 months, 12 months, 2 years, or 3 years. In some embodiments, storage for a period of time of at least 1 day is storage for 1, 3 or 7 days. In some embodiments, storage for a period of time of at least 1 day is storage for about or more than 14 or 28 days. In some embodiments, storage for a period of time of at least 1 days is storge for about or more than 8 weeks or 12 weeks. In some embodiments, storage for a period of time of at least 1 days is storage for about or more than 1 year.

In some embodiments, the concentration of polysorbate remains substantially unchanged after the formulation is stored for a prolonged period of time at 2-8° C. In some embodiments, the concentration of polysorbate remains substantially unchanged after the formulation is stored for a prolonged period of time at 25° C. In some embodiments, the concentration of polysorbate remains substantially unchanged after the formulation is stored for a prolonged period of time at 40° C. In some embodiments, the concentration of polysorbate remains substantially unchanged after the formulation is stored for a prolonged period of time at −70° C.

In some embodiments, the concentration of polysorbate remains within 40% of the starting concentration of polysorbate after storage (e.g., storage at about 2-8° C. or about 25° C., e.g., for at least 8 weeks). In some embodiments, the concentration of polysorbate remains within 30% of the starting concentration of polysorbate after storage (e.g., storage at about 2-8° C. or about 25° C., e.g., for at least 8 weeks). In some embodiments, the concentration of polysorbate remains within 20% of the starting concentration of polysorbate after storage (e.g., storage at about 2-8° C. or about 25° C., e.g., for at least 8 weeks). In some embodiments, the concentration of the polysorbate remains within 10% of the starting concentration of the polysorbate after storage (e.g., storage at about 2-8° C. or about 25° C., e.g., for at least 8 weeks).

In some embodiments, the concentration of the polysorbate remains within 5% of the starting concentration of the polysorbate after storage (e.g., storage at about 2-8° C. or about 25° C., e.g., for at least 8 weeks).

In some embodiments, the concentration of the polysorbate in the formulation remains within about 5%, within about 10%, within about 15%, within about 20%, within about 25%, within about 30%, within about 35%, within about 40%, within about 45% or within about 50% of the starting concentration after at least 28 days of storage at about 40° C. The “starting concentration” is the concentration of polysorbate directly after the formulation is filled after purification (e.g., about 0.02%).

In some embodiments, the concentration of polysorbate in the formulation remains within about 1%, within about 5%, within about 10%, within about 15%, within about 20%, within about 25%, within about 30%, within about 35%, within about 40%, within about 45% or within about 50% of the starting concentration of polysorbate after at least 8 weeks of storage at −70° C.

In some embodiments, the concentration of polysorbate in the formulation remains within about 1%, within about 5%, within about 10%, within about 15%, within about 20%, within about 25%, within about 30%, within about 35%, within about 40%, within about 45% or within about 50% of the starting concentration of polysorbate after at least 8 weeks of storage at about 2-8° C.

In some embodiments, the concentration of polysorbate in the formulation remains within about 1%, within about 5%, within about 10%, within about 15%, within about 20%, within about 25%, within about 30%, within about 35%, within about 40%, within about 45% or within about 50% of the starting concentration after at least 8 weeks of storage at about 25° C.

In some aspects, the concentration of certain species of polysorbate remains substantially constant during storage. For example, the concentration of polyesters and/or monoesters of a polysorbate may remain substantially unchanged for a prolonged period of time. In some embodiments, the degradation products are composed of free fatty acids (FFAs), primarily lauric acid for PS20 and/or POE head groups.

In some embodiments, the concentration of polyester species of a polysorbate remains within 10%, 20%, 30% or 40% of the starting concentration of the polyester species after storage for a period of time of at least 1 day. In some embodiments, the concentration of monoester species of a polysorbate remains within 10%, 20%, 30% or 40% of the starting concentration of the monoester species after storage for a period of time of at least 1 day.

In some embodiments, the concentration of polyesters and/or monoesters of a polysorbate in the formulation remains within about 5%, within about 10%, within about 15%, within about 20%, within about 25%, within about 30%, within about 35%, within about 40%, within about 45% or within about 50% of the starting concentration after at least 28 days of storage at about 40° C.

In some embodiments, the concentration of polyesters and/or monoesters of a polysorbate in the formulation remains within about 1%, within about 5%, within about 10%, within about 15%, within about 20%, within about 25%, within about 30%, within about 35%, within about 40%, within about 45% or within about 50% of the starting concentration after at least 8 weeks of storage at −70° C.

In some embodiments, the concentration of polyesters and/or monoesters of a polysorbate in the formulation remains within about 1%, within about 5%, within about 10%, within about 15%, within about 20%, within about 25%, within about 30%, within about 35%, within about 40%, within about 45% or within about 50% of the starting concentration after at least 8 weeks of storage at about 2-8° C.

In some embodiments, the concentration of polyesters and/or monoesters of a polysorbate in the formulation remains within about 1%, within about 5%, within about 10%, within about 15%, within about 20%, within about 25%, within about 30%, within about 35%, within about 40%, within about 45% or within about 50% of the starting concentration after at least 8 weeks of storage at about 25° C.

In some aspects, the amount of a degradation product of a polysorbate (e.g., lauric acid and/or non-esterified species of PS20) does not increase, or does not substantially increase, in the formulation. In some embodiments, the presence of a polysorbate degradation product does not increase by more than 50% of the starting concentration of the degradation product after storage. In some aspects, the amount of a degradation product of a polysorbate (e.g., oleic acid and/or non-esterified species of PS80) does not increase, or does not substantially increase, in the formulation. In some embodiments, the presence of a polysorbate degradation product does not increase by more than 50% of the starting concentration of the degradation product after storage. In some embodiments, the presence of a polysorbate degradation product does not increase by more than 25% of the starting concentration of the degradation product after storage. In some embodiments, the presence of a polysorbate degradation product does not increase by more than 10% of the starting concentration of the degradation product after storage.

In some embodiments, the concentration of non-esterified species of a polysorbate in the formulation remains within about 5%, within about 10%, within about 15%, within about 20%, within about 25%, within about 30%, within about 35%, within about 40%, within about 45% or within about 50% of the starting concentration after at least 28 days of storage at about 40° C.

In some embodiments, the concentration of non-esterified species of a polysorbate in the formulation remains within about 1%, within about 5%, within about 10%, within about 15%, within about 20%, within about 25%, within about 30%, within about 35%, within about 40%, within about 45% or within about 50% of the starting concentration after at least 8 weeks of storage at −70° C.

In some embodiments, the concentration of non-esterified species of a polysorbate in the formulation remains within about 1%, within about 5%, within about 10%, within about 15%, within about 20%, within about 25%, within about 30%, within about 35%, within about 40%, within about 45% or within about 50% of the starting concentration after at least 8 weeks of storage at about 2-8° C.

In some embodiments, the concentration of non-esterified species of a polysorbate in the formulation remains within about 1%, within about 5%, within about 10%, within about 15%, within about 20%, within about 25%, within about 30%, within about 35%, within about 40%, within about 45% or within about 50% of the starting concentration after at least 8 weeks of storage at about 25° C.

In some embodiments described herein, lauric acid concentration is to be measured in formulations comprising PS20, since it is prominent degradation product of PS20. In some embodiments described herein, oleic acid concentration is to be measured in formulations comprising PS80, since it is prominent degradation product of PS80.

In some embodiments, the concentration of lauric acid in the formulation remains within about 5%, within about 10%, within about 15%, within about 20%, within about 25%, within about 30%, within about 35%, within about 40%, within about 45% or within about 50% of the starting concentration after at least 28 days of storage at about 40° C.

In some embodiments, the concentration of lauric acid in the formulation remains within about 1%, within about 5%, within about 10%, within about 15%, within about 20%, within about 25%, within about 30%, within about 35%, within about 40%, within about 45% or within about 50% of the starting concentration after at least 8 weeks of storage at −70° C.

In some embodiments, the concentration of lauric acid in the formulation remains within about 1%, within about 5%, within about 10%, within about 15%, within about 20%, within about 25%, within about 30%, within about 35%, within about 40%, within about 45% or within about 50% of the starting concentration after at least 8 weeks of storage at about 2-8° C.

In some embodiments, the concentration of lauric acid in the formulation remains within about 1%, within about 5%, within about 10%, within about 15%, within about 20%, within about 25%, within about 30%, within about 35%, within about 40%, within about 45% or within about 50% of the starting concentration after at least 8 weeks of storage at about 25° C.

In some embodiments, the concentration of oleic acid in the formulation remains within about 5%, within about 10%, within about 15%, within about 20%, within about 25%, within about 30%, within about 35%, within about 40%, within about 45% or within about 50% of the starting concentration after at least 28 days of storage at about 40° C.

In some embodiments, the concentration of oleic acid in the formulation remains within about 1%, within about 5%, within about 10%, within about 15%, within about 20%, within about 25%, within about 30%, within about 35%, within about 40%, within about 45% or within about 50% of the starting concentration after at least 8 weeks of storage at −70° C.

In some embodiments, the concentration of oleic acid in the formulation remains within about 1%, within about 5%, within about 10%, within about 15%, within about 20%, within about 25%, within about 30%, within about 35%, within about 40%, within about 45% or within about 50% of the starting concentration after at least 8 weeks of storage at about 2-8° C.

In some embodiments, the concentration of oleic acid in the formulation remains within about 1%, within about 5%, within about 10%, within about 15%, within about 20%, within about 25%, within about 30%, within about 35%, within about 40%, within about 45% or within about 50% of the starting concentration after at least 8 weeks of storage at about 25° C.

In some embodiments, the formulation is at least 90%, at least 95%, at least 96%, at least 97%, at least 98%, at least 99%, at least 99.5%, at least 99.8%, at least 99.9% or 100% free of impurities.

In some embodiments, the formulation is at least 90%, at least 95%, at least 96%, at least 97%, at least 98%, at least 99%, at least 99.5%, at least 99.8%, at least 99.9% or 100% free of host cell proteins (e.g., proteins from the cell in which the protein having an Fc region is produced). In some embodiments, the formulations described herein comprise host cell proteins at a concentration of less than 20 pg/mg, less than 15 pg/mg, less than 10 pg/mg, less than 7.5 pg/mg, less than 5 pg/mg, less than 4 pg/mg, less than 3 pg/mg, less than 2.5 pg/mg, less than 2 pg/mg, less than 1.5 pg/mg, or less than 1 pg/mg. In some embodiments, the formulations described herein comprise host cell proteins at a concentration of less than 5 pg/mg or less than 2 pg/mg. In some embodiments, host cell proteins are undetectable in the formulations described herein. Any methods of detection of host cell proteins described herein or known in the art can be used. In some embodiments, the formulation is at least 90%, at least 95%, at least 96%, at least 97%, at least 98%, at least 99%, at least 99.5%, at least 99.8%, at least 99.9% or 100% free of host cell lipases and/or hydrolases. In some embodiments, the formulation is at least 90%, at least 95%, at least 96%, at least 97%, at least 98%, at least 99%, at least 99.5%, at least 99.8%, at least 99.9% or 100% free of host cell proteins with lipase activity. In some embodiments, host cell proteins with lipase activity are undetectable in the formulations described herein. In some embodiments, the formulation is at least 90%, at least 95%, at least 96%, at least 97%, at least 98%, at least 99%, at least 99.5%, at least 99.8%, at least 99.9% or 100% free of host cell proteins with hydrolase activity. In some embodiments, host cell proteins with hydrolase activity are undetectable in the formulations described herein. In some embodiments, the formulation is at least 90%, at least 95%, at least 96%, at least 97%, at least 98%, at least 99%, at least 99.5%, at least 99.8%, at least 99.9% or 100% free of host cell proteins with esterase activity. In some embodiments, host cell proteins with esterase activity are undetectable in the formulations described herein. Lipase, esterase and/or hydrolase activity may be determined by measuring the stability of polysorbates.

In some embodiments, the formulation is at least 90%, at least 95%, at least 96%, at least 97%, at least 98%, at least 99%, at least 99.5%, at least 99.8%, at least 99.9% or 100% free of host cell DNA (e.g., DNA from the cells in which the protein having an Fc region is produced). In some embodiments, the formulations described herein comprise host cell DNA at a concentration of less than 2 pg/mg, less than 1.5 pg/mg, less than 1 pg/mg, less than 0.75 pg/mg, less than 0.5 pg/mg, less than 0.4 pg/mg, less than 0.3 pg/mg, less than 0.25 pg/mg, less than 0.2 pg/mg, less than 0.15 pg/mg, less than 0.1 pg/mg, or less than 0.05 pg/mg. In some embodiments, the formulations described herein comprise host cell proteins at a concentration of less than 0.5 pg/mg or less than 0.2 pg/mg. In some embodiments, host cell DNA is undetectable in the formulations described herein. Any methods of detection of host cell DNA described herein or known in the art can be used.

In some embodiments, the formulations described herein do not comprise an antioxidant. In some embodiments, the formulation does not comprise BHA. In some embodiments, the formulation does not comprise BHT. In some embodiments, the formulations described herein do not comprise a lipase inhibitor. In other embodiments, the formulations described herein further comprise an antioxidant. In some embodiments, the formulations described herein further comprise a lipase inhibitor.

Methods of Producing Protein Formulations with Improved Polysorbate Stability.

In another aspect, provided herein are methods of purifying a protein having an Fc region that results in improved surfactant (such as polysorbate) stability in the final formulation of the protein having an Fc region, as well as protein formulations produced by such methods. In some embodiments, the polysorbate is PS20. In some embodiments, the polysorbate is PS80.

In some embodiments, the formulation produced by a method described herein comprises a protein having an Fc region at a high concentration, for example, a concentration of about 50-100 g/L, about 100-110 g/L, about 110-120 g/L, about 120-130 g/L, about 130-140 g/L, about 140-150 g/L, about 150-160 g/L, about 160-170 g/L, about 170-180 g/L, about 180-190 g/L, or about 190-200 g/L. In some embodiments, the formulation produced by a method described herein comprises a protein having an Fc region at a concentration of about 50 g/L, about 100 g/L, about 110 g/L, about 120 g/L, about 130 g/L, about 140 g/L, about 150 g/L, about 160 g/L, about 170 g/L, about 180 g/L, about 190 g/L, or about 200 g/L. In some embodiments, the formulation produced by a method described herein comprises 170 mg/mL anti-FcRn antibody in 100 mM L-Histidine/Histidine HCl, 100 mM L-Arginine HCl and 0.02% Polysorbate 20 in water at pH 6. In some embodiments, the formulation produced by a method described herein may be for injection, e.g., subcutaneous injection.

Proteins, including proteins having an Fc domain described herein, may be produced by a fed-batch cell culture process, using commercially available, animal-component-free media in a bioreactor. Proteins, including proteins having an Fc domain described herein, may be isolated from any suitable host cell, such as any suitable mammalian cell, including, for example, CHO cells, NS0 cells, Sp2/0 cells, HEK293 cells and PERC6 cells. Cells expressing proteins, e.g., a protein having an Fc domain described herein, may be generated using any suitable method known in the art. For example, cells maybe transfected with a vector expressing a protein having an Fc region. The cells may be harvested at a suitable time point and the protein may be purified from the cell as described herein or using any suitable method known in the art.

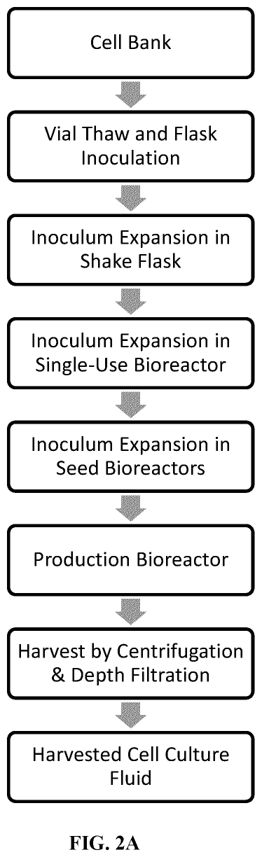

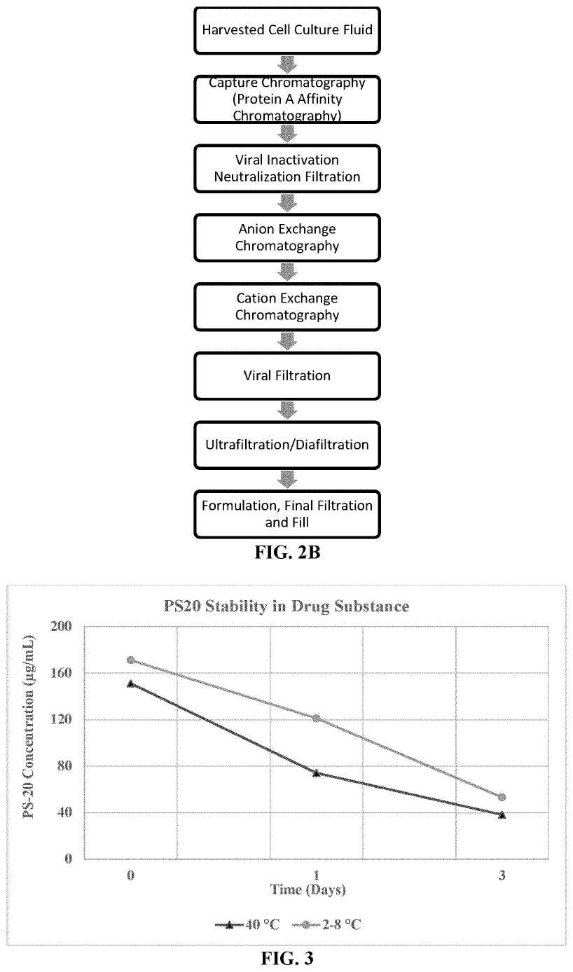

An overview of the upstream process is shown in FIG. 2A. Generally, the upstream process comprises thawing of a cell bank vial comprising cells engineered to express the protein having an Fc region, inoculum expansion of the cells in flasks, and then further expansion in a production bioreactor. The cell culture harvested from the production bioreactor is clarified using centrifugation, followed by depth filtration and bioburden reduction. The resulting harvest comprises the protein having an Fc region and is the starting solution for further purification.

The downstream purification process is illustrated in FIG. 2B. Generally, the downstream process comprises chromatography to capture the protein having an Fc region from the clarified harvest. Any Fc-binding resin may be used in this chromatography step, including, for example, Protein A, Protein G, or a resin having genetically engineered Fc binding proteins. Examples of Fc-binding resins that can be used in the methods described herein are provided in Lei et al., December 2022, TrAC Trends in Analytical Chemistry Volume 157, p. 116744, which is incorporated herein by reference in its entirety for examples of chromatography resins that may be used in the methods described herein.

The chromatography step may be followed by low pH viral inactivation, Anion Exchange (AEX) Chromatography, and/or Cation Exchange (CEX) Chromatography. The resulting pool may then be filtered through, e.g., a nanofilter and/or ultrafiltered and diafiltered to generate concentrated and buffer exchanged UFDF pool. The recovered filtration pool may then be formulated.

In some embodiments, the method of purifying a protein having an Fc region from harvested cell culture comprises: (i) loading the harvested cell culture comprising the protein having an Fc region on to an Fc-binding affinity resin chromatography column, (ii) washing the column one, two, three or more times using one or more wash buffers, wherein at least one wash buffer comprises a pH of greater than 9, and (iii) eluting the protein having an Fc region from the column; optionally wherein the method is for making a high concentration formulation of the protein with one or more excipients comprising a polysorbate.

In some embodiments, the method comprises (i) loading cell culture comprising the protein having an Fc region on to an Fc-binding affinity resin chromatography column (e.g., Protein A chromatography column); (ii) washing the column one, two, three or more times using one or more wash buffers, wherein at least one wash buffer comprises a pH of greater than 9 (or equal to or greater than 10); (iii) eluting the protein having an Fc region from the column; (iv) optionally subjecting the eluate from step (iii), comprising the protein having an Fc region, to filtration and/or concentration; and (v) formulating the protein having an Fc region from step (iv) into a pharmaceutical formulation comprising adding one or more excipients, wherein the concentration of the protein having an Fc region in the pharmaceutical formulation is higher than 50 g/L or higher than or equal to 100 g/L, and wherein the one or more excipients comprise a polysorbate (e.g., PS20 or PS80).

In some embodiments, the method comprises (i) loading cell culture comprising the protein having an Fc region on to a Protein A chromatography column; (ii) washing the column one, two, three or more times using one or more wash buffers, wherein at least one wash buffer comprises a pH of equal to or greater than 10; (iii) eluting the protein having an Fc region from the column; (iv) optionally subjecting the eluate from step (iii), comprising the protein having an Fc region, to filtration and/or concentration; and (v) formulating the protein having an Fc region from step (iv) into a pharmaceutical formulation comprising adding one or more excipients, wherein the concentration of the protein having an Fc region in the pharmaceutical formulation is higher than 75 g/L or higher than or equal to 100 g/L, and wherein the one or more excipients comprise Polysorbate 20.

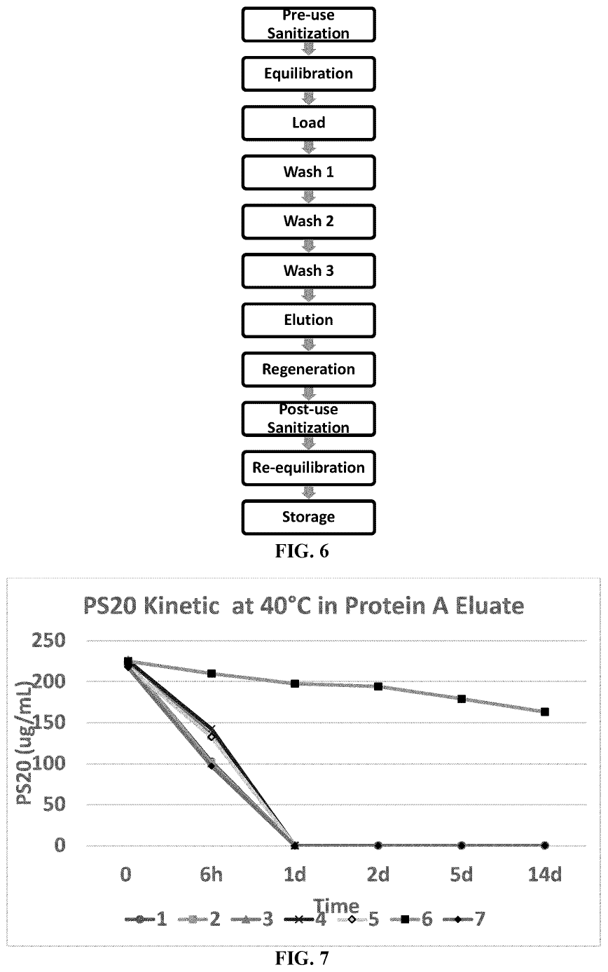

Chromatography comprises several steps, including, for example, equilibrating a column packed with MabSelect SuRe LX resin with an equilibration buffer prior to loading the solution comprising the protein having an Fc region onto the column, and washing the column with three different buffers (Wash 1, Wash 2 & Wash 3) prior to elution. The stringency of the washes increases from Wash 1 to Wash 2, and the column is prepared for elution by Wash 3. The protein having an Fc region is then eluted using a low pH elution buffer. See FIG. 6 for an illustration for a Protein A chromatography process. Further details of the Protein A chromatography process are described in the Examples section below.

Without wishing to be bound by theory, it is believed that the chromatography step is essential in determining surfactant (such as polysorbate) stability in the final protein formulation. In particular, the pH of the buffer used in the second wash step (“wash 2 buffer”) is believed to be important in determining polysorbate stability. In some embodiments, the wash 2 buffer is a high-pH buffer. In some embodiments, the wash 2 buffer has a pH greater than 9. In some embodiments the wash 2 buffer has a pH of about or more than 9.5. In some embodiments, the wash 2 buffer has a pH of about or more than 10. In some embodiments, the wash 2 buffer has a pH of at least 10. In some embodiments, the wash 2 buffer has a pH of about or more than 10.5. In some embodiments, the wash 2 buffer has a pH of about or more than 11. In some embodiments the wash 2 buffer has a pH of about or more than 11.5.

Without wishing to be bound by theory, it is believed that the chromatography step with a high pH wash buffer may disrupt non-covalent or covalent interactions between a protein having an Fc region (purified in accordance with the methods described herein) and host cell proteins (such as lipases, esterases and/or hydrolases).

Another factor that may contribute to the stability of polysorbate in the final protein formulation is the column load ratio. Thus, it is believed that a lower column load ratio in the chromatography step improves polysorbate stability. In some embodiments, the Protein A column load ratio is 40 g/Lᵣₑₛᵢₙ or less. In some embodiments, the Protein A column load ratio is 35 g/Lᵣₑₛᵢₙ or less. In some embodiments, the Protein A column load ratio is 30 g/Lᵣₑₛᵢₙ or less. In some embodiments, the column load ratio is 25.0 g/Lᵣₑₛᵢₙ or less. In some embodiments, the column load ratio is 20 g/Lᵣₑₛᵢₙ or less. In some embodiments, the column load ratio is 15 g/Lᵣₑₛᵢₙ or less. In some embodiments, the Protein A column load ratio is about 5 g/Lᵣₑₛᵢₙ to about 10 g/Lᵣₑₛᵢₙ. In some embodiments, the Protein A column load ratio is about 10 g/Lᵣₑₛᵢₙ to about 15 g/Lᵣₑₛᵢₙ. In some embodiments, the Protein A column load ratio is about 15 g/Lᵣₑₛᵢₙ to about 20 g/Lᵣₑₛᵢₙ. In some embodiments, the Protein A column load ratio is about 20 g/Lᵣₑₛᵢₙ to about 25 g/Lᵣₑₛᵢₙ. In some embodiments, the Protein A column load ratio is about 25 g/Lᵣₑₛᵢₙ to about 30 g/Lᵣₑₛᵢₙ. In some embodiments, the Protein A column load ratio is about 30 g/Lᵣₑₛᵢₙ to about 35 g/Lᵣₑₛᵢₙ. In some embodiments, the Protein A column load ratio is about 35 g/Lᵣₑₛᵢₙ to about 40 g/Lᵣₑₛᵢₙ. In some embodiments, the Protein A column load ratio is about 40 g/Lᵣₑₛᵢₙ to about 45 g/Lᵣₑₛᵢₙ. In some embodiments, the Protein A column load ratio is about 45 g/Lᵣₑₛᵢₙ to about 50 g/Lᵣₑₛᵢₙ.

In some embodiments, the Protein A column load ratio is about 1 g/Lᵣₑₛᵢₙ. In some embodiments, the Protein A column load ratio is about 2 g/Lᵣₑₛᵢₙ. In some embodiments, the Protein A column load ratio is about 3 g/Lᵣₑₛᵢₙ. In some embodiments, the Protein A column load ratio is about 4 g/Lᵣₑₛᵢₙ. In some embodiments, the Protein A column load ratio is about 5 g/Lᵣₑₛᵢₙ. In some embodiments, the Protein A column load ratio is about 10 g/Lᵣₑₛᵢₙ. In some embodiments, the Protein A column load ratio is about 15 g/Lᵣₑₛᵢₙ. In some embodiments, the Protein A column load ratio is about 20 g/Lᵣₑₛᵢₙ.

In some embodiments, the wash 2 buffer comprises a high concentration of salt, for example, sodium chloride (NaCl). In some embodiments, the wash 2 buffer comprises at least 0.5 M NaCl. In Some embodiments, the wash 2 buffer comprises about 1 M NaCl. In some embodiments, the wash 2 buffer does not comprise a high concentration of salt (e.g., 0.3 M NaCl or less).

In some embodiments, the wash 2 buffer comprises Arginine. In some embodiments, the wash 2 buffer comprises about 0.5 M Arginine. In some embodiments, the wash 2 buffer does not comprise Arginine.

The purification methods described herein may comprise further steps after the chromatography step. For example, the method of purification may further comprise one or more viral inactivation steps. Methods of viral inactivation are known in the art.

In some embodiments, the methods described herein comprise at least one additional chromatography step. In some embodiments, the at least one additional chromatography step is Anion Exchange (AEX) chromatography. AEX chromatography can remove impurities, such as protein size variants, host cell proteins, residual Protein A, and/or host cell DNA. Methods for AEX chromatography are known in the art. In some embodiments, the at least one additional chromatography step is Cation Exchange (CEX) Chromatography. CEX chromatography can further remove impurities, such as protein size variants, host cell protein, residual resin from the chromatography column, and/or host cell DNA. Methods for CEX chromatography are known in the art.

In some embodiments, the methods described herein comprise further comprises at least one filtration step. For example, the method may further comprise a viral filtration step. Viral filtration sterically removes both enveloped and non-enveloped viruses. Methods of vial filtration are known in the art.

In some embodiments, the methods described herein further comprise an ultrafiltration and/or diafiltration step. In some embodiments, the methods described herein further comprise an ultrafiltration/diafiltration (UFDF) step. UFDF may be used to exchange the buffer of the solution to the diafiltration buffer and to concentrate the protein having an Fc region to a target concentration. Methods of UFDF are known in the art,

In another aspect, provided herein is a formulation comprising a protein having an Fc region produced by any of the methods described herein.

In some embodiments, the formulations produced by a method described herein comprises a polysorbate. In some embodiments, the polysorbate is PS20. In some embodiments, the polysorbate is PS80.

In some embodiments, the concentration of the polysorbate remains within 30% of the starting concentration of the polysorbate after storage for a period of time of at least 1 day and/or wherein the presence of a polysorbate degradation product does not increase by more than 2 times of the starting concentration of the degradation product after storage for a period of time of at least 1 day. In some embodiments, storage for a period of time of at least 1 day is storage for 1, 3 or 7 days.

In some embodiments, storage for a period of time of at least 1 day is storage for about or more than 14 or 28 days. In some embodiments, storage for a period of time of at least 1 days is storge for about or more than 8 weeks or 12 weeks. In some embodiments, storage for a period of time of at least 1 days is storage for about or more than 1 year.

In some embodiments, the concentration of the polysorbate remains within 20% of the starting concentration of the polysorbate after storage. In some embodiments, the concentration of the polysorbate remains within 10% of the starting concentration of the polysorbate after storage. In some embodiments, the concentration of the polysorbate remains within 5% of the starting concentration of the polysorbate after storage.

In some embodiments, the concentration of polysorbate in the formulation produced by a method described herein remains within about 5%, within about 10%, within about 15%, within about 20%, within about 25%, within about 30%, within about 35%, within about 40%, within about 45% or within about 50% of the starting concentration after at least 21 days of storage at about 40° C.

In some embodiments, the concentration of polysorbate in the formulation produced by a method described herein remains within about 5%, within about 10%, within about 15%, within about 20%, within about 25%, within about 30%, within about 35%, within about 40%, within about 45% or within about 50% of the starting concentration after at least 28 days of storage at about 40° C.

In some embodiments, the concentration of polysorbate in the formulation produced by a method described herein remains within about 5%, within about 10%, within about 15%, within about 20%, within about 25%, within about 30%, within about 35%, within about 40%, within about 45% or within about 50% of the starting concentration after at least 30 days of storage at about 40° C.

In some embodiments, the concentration of polysorbate in the formulation produced by a method described herein remains within about 1%, within about 5%, within about 10%, within about 15%, within about 20%, within about 25%, within about 30%, within about 35%, within about 40%, within about 45% or within about 50% of the starting concentration after at least 8 weeks of storage at −70° C.

In some embodiments, the concentration of polysorbate in the formulation produced by a method described herein remains within about 1%, within about 5%, within about 10%, within about 15%, within about 20%, within about 25%, within about 30%, within about 35%, within about 40%, within about 45% or within about 50% of the starting concentration after at least 21 days of storage at about 2-8° C.

In some embodiments, the concentration of polysorbate in the formulation produced by a method described herein remains within about 1%, within about 5%, within about 10%, within about 15%, within about 20%, within about 25%, within about 30%, within about 35%, within about 40%, within about 45% or within about 50% of the starting concentration after at least 30 days of storage at about 2-8° C.

In some embodiments, the concentration of polysorbate in the formulation produced by a method described herein remains within about 1%, within about 5%, within about 10%, within about 15%, within about 20%, within about 25%, within about 30%, within about 35%, within about 40%, within about 45% or within about 50% of the starting concentration after at least 8 weeks of storage at about 2-8° C.

In some embodiments, the concentration of polysorbate in the formulation produced by a method described herein remains within about 1%, within about 5%, within about 10%, within about 15%, within about 20%, within about 25%, within about 30%, within about 35%, within about 40%, within about 45% or within about 50% of the starting concentration after at least 8 weeks of storage at about 25° C.

In some embodiments, the concentration of the concentration of polyesters and/or monoesters of a polysorbate in the formulation produced by a method described herein remains within about 5%, within about 10%, within about 15%, within about 20%, within about 25%, within about 30%, within about 35%, within about 40%, within about 45% or within about 50% of the starting concentration after at least 28 days of storage at about 40° C. In some embodiments, the concentration of polyester species of a polysorbate remains within 10% or 20% of the starting concentration of the polyester species after storage for a period of time of at least 1 day. In some embodiments, the concentration of monoester species of a polysorbate remains within 10% or 20% of the starting concentration of the monoester species after storage for a period of time of at least 1 day.

In some embodiments, the concentration of the concentration of polyesters and/or monoesters of a polysorbate in the formulation produced by a method described herein remains within about 5%, within about 10%, within about 15%, within about 20%, within about 25%, within about 30%, within about 35%, within about 40%, within about 45% or within about 50% of the starting concentration after at least 21 days of storage at about 40° C.

In some embodiments, the concentration of the concentration of polyesters and/or monoesters of a polysorbate in the formulation produced by a method described herein remains within about 5%, within about 10%, within about 15%, within about 20%, within about 25%, within about 30%, within about 35%, within about 40%, within about 45% or within about 50% of the starting concentration after at least 30 days of storage at about 40° C.

In some embodiments, the concentration of polyesters and/or monoesters of a polysorbate in the formulation produced by a method described herein remains within about 1%, within about 5%, within about 10%, within about 15%, within about 20%, within about 25%, within about 30%, within about 35%, within about 40%, within about 45% or within about 50% of the starting concentration after at least 8 weeks of storage at −70° C.

In some embodiments, the concentration of polyesters and/or monoesters of a polysorbate in the formulation produced by a method described herein remains within about 1%, within about 5%, within about 10%, within about 15%, within about 20%, within about 25%, within about 30%, within about 35%, within about 40%, within about 45% or within about 50% of the starting concentration after at least 21 days of storage at about 2-8° C.

In some embodiments, the concentration of polyesters and/or monoesters of a polysorbate in the formulation produced by a method described herein remains within about 1%, within about 5%, within about 10%, within about 15%, within about 20%, within about 25%, within about 30%, within about 35%, within about 40%, within about 45% or within about 50% of the starting concentration after at least 30 days of storage at about 2-8° C.

In some embodiments, the concentration of polyesters and/or monoesters of a polysorbate in the formulation produced by a method described herein remains within about 1%, within about 5%, within about 10%, within about 15%, within about 20%, within about 25%, within about 30%, within about 35%, within about 40%, within about 45% or within about 50% of the starting concentration after at least 8 weeks of storage at about 25° C.

In some aspects, the amount of non-esterified species of a polysorbate (a degradation product) does not increase in the formulation produced by a method described herein. In some embodiments, the presence of a degradation product of a polysorbate does not increase by more than 25% of the starting concentration of the degradation product after storage. In some embodiments, the presence of a degradation product of a polysorbate does not increase by more than 10% of the starting concentration of the degradation product after storage. In some embodiments, the degradation product of a polysorbate is a non-esterified species of the polysorbate. In some embodiments, the degradation product is a non-esterified species of Polysorbate 20. In some embodiments, the non-esterified species of Polysorbate 20 is Sorbitan or Isosorbide. In some embodiments, the Polysorbate 20 degradation product is Lauric acid. In some embodiments, the presence of a Polysorbate 20 degradation product does not increase by more than 50% of the starting concentration of the degradation product after storage. In some embodiments, the degradation product is a non-esterified species of Polysorbate 80. In some embodiments, the Polysorbate 80 degradation product is Oleic acid. In some embodiments, the presence of a Polysorbate 80 degradation product does not increase by more than 50% of the starting concentration of the degradation product after storage.

In some embodiments, the concentration of non-esterified species of a polysorbate in the formulation produced by a method described herein remains within about 5%, within about 10%, within about 15%, within about 20%, within about 25%, within about 30%, within about 35%, within about 40%, within about 45% or within about 50% of the starting concentration after at least 21 days of storage at about 40° C.

In some embodiments, the concentration of non-esterified species of a polysorbate in the formulation produced by a method described herein remains within about 5%, within about 10%, within about 15%, within about 20%, within about 25%, within about 30%, within about 35%, within about 40%, within about 45% or within about 50% of the starting concentration after at least 28 days of storage at about 40° C.

In some embodiments, the concentration of non-esterified species of a polysorbate in the formulation produced by a method described herein remains within about 5%, within about 10%, within about 15%, within about 20%, within about 25%, within about 30%, within about 35%, within about 40%, within about 45% or within about 50% of the starting concentration after at least 30 days of storage at about 40° C.

In some embodiments, the concentration of non-esterified species of a polysorbate in the formulation produced by a method described herein remains within about 1%, within about 5%, within about 10%, within about 15%, within about 20%, within about 25%, within about 30%, within about 35%, within about 40%, within about 45% or within about 50% of the starting concentration after at least 8 weeks of storage at −70° C.

In some embodiments, the concentration of non-esterified species of PS20 in the formulation produced by a method described herein remains within about 1%, within about 5%, within about 10%, within about 15%, within about 20%, within about 25%, within about 30%, within about 35%, within about 40%, within about 45% or within about 50% of the starting concentration after at least 21 days of storage at about 2-8° C.

In some embodiments, the concentration of non-esterified species of PS20 in the formulation produced by a method described herein remains within about 1%, within about 5%, within about 10%, within about 15%, within about 20%, within about 25%, within about 30%, within about 35%, within about 40%, within about 45% or within about 50% of the starting concentration after at least 30 days of storage at about 2-8° C.

In some embodiments, the concentration of non-esterified species of PS20 in the formulation produced by a method described herein remains within about 1%, within about 5%, within about 10%, within about 15%, within about 20%, within about 25%, within about 30%, within about 35%, within about 40%, within about 45% or within about 50% of the starting concentration after at least 8 weeks of storage at about 25° C.

In some embodiments, the concentration of lauric acid in the formulation produced by a method described herein remains within about 5%, within about 10%, within about 15%, within about 20%, within about 25%, within about 30%, within about 35%, within about 40%, within about 45% or within about 50% of the starting concentration after at least 21 days of storage at about 40° C.

In some embodiments, the concentration of lauric acid in the formulation produced by a method described herein remains within about 5%, within about 10%, within about 15%, within about 20%, within about 25%, within about 30%, within about 35%, within about 40%, within about 45% or within about 50% of the starting concentration after at least 28 days of storage at about 40° C.

In some embodiments, the concentration of lauric acid in the formulation produced by a method described herein remains within about 5%, within about 10%, within about 15%, within about 20%, within about 25%, within about 30%, within about 35%, within about 40%, within about 45% or within about 50% of the starting concentration after at least 30 days of storage at about 40° C.

In some embodiments, the concentration of lauric acid in the formulation produced by a method described herein remains within about 1%, within about 5%, within about 10%, within about 15%, within about 20%, within about 25%, within about 30%, within about 35%, within about 40%, within about 45% or within about 50% of the starting concentration after at least 8 weeks of storage at −70° C.

In some embodiments, the concentration of lauric acid in the formulation produced by a method described herein remains within about 1%, within about 5%, within about 10%, within about 15%, within about 20%, within about 25%, within about 30%, within about 35%, within about 40%, within about 45% or within about 50% of the starting concentration after at least 21 days of storage at about 2-8° C.

In some embodiments, the concentration of lauric acid in the formulation produced by a method described herein remains within about 1%, within about 5%, within about 10%, within about 15%, within about 20%, within about 25%, within about 30%, within about 35%, within about 40%, within about 45% or within about 50% of the starting concentration after at least 30 days of storage at about 2-8° C.

In some embodiments, the concentration of lauric acid in the formulation produced by a method described herein remains within about 1%, within about 5%, within about 10%, within about 15%, within about 20%, within about 25%, within about 30%, within about 35%, within about 40%, within about 45% or within about 50% of the starting concentration after at least 8 weeks of storage at about 2-8° C.

In some embodiments, the concentration of lauric acid in the formulation produced by a method described herein remains within about 1%, within about 5%, within about 10%, within about 15%, within about 20%, within about 25%, within about 30%, within about 35%, within about 40%, within about 45% or within about 50% of the starting concentration after at least 8 weeks of storage at about 25° C.

In some embodiments, the concentration of non-esterified species of PS80 in the formulation produced by a method described herein remains within about 1%, within about 5%, within about 10%, within about 15%, within about 20%, within about 25%, within about 30%, within about 35%, within about 40%, within about 45% or within about 50% of the starting concentration after at least 21 days of storage at about 2-8° C.

v In some embodiments, the concentration of non-esterified species of PS80 in the formulation produced by a method described herein remains within about 1%, within about 5%, within about 10%, within about 15%, within about 20%, within about 25%, within about 30%, within about 35%, within about 40%, within about 45% or within about 50% of the starting concentration after at least 30 days of storage at about 2-8° C.

In some embodiments, the concentration of non-esterified species of PS80 in the formulation produced by a method described herein remains within about 1%, within about 5%, within about 10%, within about 15%, within about 20%, within about 25%, within about 30%, within about 35%, within about 40%, within about 45% or within about 50% of the starting concentration after at least 8 weeks of storage at about 2-8° C.

In some embodiments, the concentration of non-esterified species of PS80 in the formulation produced by a method described herein remains within about 1%, within about 5%, within about 10%, within about 15%, within about 20%, within about 25%, within about 30%, within about 35%, within about 40%, within about 45% or within about 50% of the starting concentration after at least 8 weeks of storage at about 25° C.

In some embodiments, the concentration of oleic acid in the formulation produced by a method described herein remains within about 5%, within about 10%, within about 15%, within about 20%, within about 25%, within about 30%, within about 35%, within about 40%, within about 45% or within about 50% of the starting concentration after at least 21 days of storage at about 40° C.

In some embodiments, the concentration of oleic acid in the formulation produced by a method described herein remains within about 5%, within about 10%, within about 15%, within about 20%, within about 25%, within about 30%, within about 35%, within about 40%, within about 45% or within about 50% of the starting concentration after at least 28 days of storage at about 40° C.

In some embodiments, the concentration of oleic acid in the formulation produced by a method described herein remains within about 5%, within about 10%, within about 15%, within about 20%, within about 25%, within about 30%, within about 35%, within about 40%, within about 45% or within about 50% of the starting concentration after at least 30 days of storage at about 40° C.

In some embodiments, the concentration of oleic acid in the formulation produced by a method described herein remains within about 1%, within about 5%, within about 10%, within about 15%, within about 20%, within about 25%, within about 30%, within about 35%, within about 40%, within about 45% or within about 50% of the starting concentration after at least 8 weeks of storage at −70° C.

In some embodiments, the concentration of oleic acid in the formulation produced by a method described herein remains within about 1%, within about 5%, within about 10%, within about 15%, within about 20%, within about 25%, within about 30%, within about 35%, within about 40%, within about 45% or within about 50% of the starting concentration after at least 21 days of storage at about 2-8° C.

In some embodiments, the concentration of oleic acid in the formulation produced by a method described herein remains within about 1%, within about 5%, within about 10%, within about 15%, within about 20%, within about 25%, within about 30%, within about 35%, within about 40%, within about 45% or within about 50% of the starting concentration after at least 30 days of storage at about 2-8° C.

In some embodiments, the concentration of oleic acid in the formulation produced by a method described herein remains within about 1%, within about 5%, within about 10%, within about 15%, within about 20%, within about 25%, within about 30%, within about 35%, within about 40%, within about 45% or within about 50% of the starting concentration after at least 8 weeks of storage at about 2-8° C.

In some embodiments, the concentration of oleic acid in the formulation produced by a method described herein remains within about 1%, within about 5%, within about 10%, within about 15%, within about 20%, within about 25%, within about 30%, within about 35%, within about 40%, within about 45% or within about 50% of the starting concentration after at least 8 weeks of storage at about 25° C.

In another aspect, a method described herein reduces impurities in the formulation produced by a method described herein. In some embodiments, a formulation produced by a method described herein is at least 90%, at least 95%, at least 96%, at least 97%, at least 98%, at least 99%, at least 99.5%, at least 99.8%, at least 99.9% or 100% free of impurities.

In some embodiments, a method described herein results in removal of host cell proteins (e.g., proteins from the cells in which the antibody was produced). In some embodiments, a method described herein results in removal of host cell lipases and/or hydrolases. In some embodiments, a formulation produced by a method described herein is at least 90%, at least 95%, at least 96%, at least 97%, at least 98%, at least 99%, at least 99.5%, at least 99.8%, at least 99.9% or 100% free of host cell lipases and/or hydrolases. In some embodiments, a formulation produced by a method described herein is at least 90%, at least 95%, at least 96%, at least 97%, at least 98%, at least 99%, at least 99.5%, at least 99.8%, at least 99.9% or 100% free of host cell proteins. In some embodiments, a formulation produced by a method described herein comprises host cell proteins at a concentration of less than 20 pg/mg, less than 15 pg/mg, less than 10 pg/mg, less than 7.5 pg/mg, less than 5 pg/mg, less than 4 pg/mg, less than 3 pg/mg, less than 2.5 pg/mg, less than 2 pg/mg, less than 1.5 pg/mg, or less than 1 pg/mg. In some embodiments, a formulation produced by a method described herein comprises host cell proteins at a concentration of less than 5 pg/mg or less than 2 pg/mg. In some embodiments, host cell proteins are undetectable in the formulations produced by the methods described herein.

In some embodiments, there are no or substantially no host cell proteins with lipase and/or hydrolase activity present in the formulation. In some embodiments, host cell proteins with lipase and/or hydrolase activity are reduced by about 20%, about 30%, about 40%, about 50%, about 60%, about 70%, about 80%, about 95%, about 98%, about 99%, or about 100% relative to a formulations produced using a buffer of pH 9 or less in the chromatography wash step (e.g., the wash 2 step).

In some embodiments, a method described herein results in removal of host cell DNA (e.g., DNA from the cells in which the antibody was produced). In some embodiments, a formulation produced by a method described herein is at least 90%, at least 95%, at least 96%, at least 97%, at least 98%, at least 99%, at least 99.5%, at least 99.8%, at least 99.9% or 100% free of host cell DNA. In some embodiments, a formulation produced by a method described herein comprises host cell DNA at a concentration of less than 2 pg/mg, less than 1.5 pg/mg, less than 1 pg/mg, less than 0.75 pg/mg, less than 0.5 pg/mg, less than 0.4 pg/mg, less than 0.3 pg/mg, less than 0.25 pg/mg, less than 0.2 pg/mg, less than 0.15 pg/mg, less than 0.1 pg/mg, or less than 0.05 pg/mg. In some embodiments, a formulation produced by a method described herein comprises host cell proteins at a concentration of less than 0.5 pg/mg or less than 0.2 pg/mg. In some embodiments, host cell DNA is undetectable in the formulations produced by the methods described herein.

### ILLUSTRATIVE EMBODIMENTS I

1. A pharmaceutical formulation comprising (i) an antibody (or another protein having an fc region) at a concentration of higher than 50 g/L, and (ii) a pharmaceutically acceptable carrier, wherein the pharmaceutically acceptable carrier comprises Polysorbate 20, wherein the concentration of the Polysorbate 20 remains within 30% of the starting concentration of the Polysorbate 20 after storage for a period of time of at least 1 day and/or wherein the presence of a Polysorbate 20 degradation product does not increase by more than 2 times of the starting concentration of the degradation product after storage for a period of time of at least 1 day.
2. The pharmaceutical formulation of embodiment 1, wherein the concentration of higher than 50 mg/ml is a concentration equal to or higher than 100 g/L, 150 g/L, 170 g/L, or 200 g/L.
3. The pharmaceutical formulation of embodiment 1 or 2, wherein the antibody is a humanized or fully human monoclonal antibody.
4. The pharmaceutical formulation of any one of embodiments 1-3, wherein the antibody is an IgG1 antibody.
5. The pharmaceutical formulation of any one of embodiments 1-4, wherein the antibody is an anti-FcRn antibody that specifically binds to FcRn.
6. The pharmaceutical formulation of embodiment 5, wherein the anti-FcRn antibody is a fully human monoclonal IgG1 antibody.
7. The pharmaceutical formulation of embodiment 5 or 6, wherein the anti-FcRn antibody comprises
(a) (i) three heavy chain CDR amino acid sequences of SEQ ID No: 27 (HCDR1), SEQ ID No: 28 (HCDR2), SEQ ID No: 29 (HCDR3); and (ii) three light chain CDR amino acid sequences of SEQ ID No: 30 (LCDR1), SEQ ID No: 31 (LCDR2), SEQ ID No: 32 (LCDR3); or
(b) (i) three heavy chain CDR amino acid sequences of SEQ ID No: 49 (HCDR1), SEQ ID No: 22 (HCDR2), SEQ ID No: 23 (HCDR3); and (ii) three light chain CDR amino acid sequences of SEQ ID No: 50 (LCDR1), SEQ ID No: 25 (LCDR2), SEQ ID No: 26 (LCDR3).
   8. The pharmaceutical formulation of any one of embodiments 5-7, wherein the anti-FcRn antibody comprises
(a) a heavy chain variable region amino acid sequence of SEQ ID No: 6 and a light chain variable region amino acid sequence of SEQ ID No: 16; or
(b) a heavy chain amino acid sequence of SEQ ID No: 51; and a light chain amino acid sequence of SEQ ID No: 52.
   9. The pharmaceutical formulation of any one of embodiments 1-8, wherein the Polysorbate 20 is at a concentration from about 0.002% to 0.2%.
   10. The pharmaceutical formulation of any one of embodiments 1-8, wherein the Polysorbate 20 is at a concentration from 0.01 to 0.05%.
   11. The pharmaceutical formulation of any one of embodiments 1-10, wherein the antibody is an anti-FcRn antibody that specifically binds to FcRn, and wherein the pharmaceutical formulation comprises at least or about 170 g/L of the anti-FcRn antibody, 0.02% Polysorbate 20, 100 mM L-Histidine/Histidine HCl and 100 mM L-Arginine HCl in water at pH 6.0.
   12. The pharmaceutical formulation of any one of embodiments 1-11, wherein the concentration of the Polysorbate 20 remains within 20% of the starting concentration of the Polysorbate 20 after storage.
   13. The pharmaceutical formulation of any one of embodiments 1-11, wherein the concentration of the Polysorbate 20 remains within 10% of the starting concentration of the Polysorbate 20 after storage.
   14. The pharmaceutical formulation of any one of embodiments 1-11, wherein the concentration of the Polysorbate 20 remains within 5% of the starting concentration of the Polysorbate 20 after storage.
   15. The pharmaceutical formulation of any one of embodiments 1-14, wherein the Polysorbate 20 degradation product is a non-esterified species of Polysorbate 20.
   16. The pharmaceutical formulation of embodiment 15, wherein the non-esterified species of Polysorbate 20 is Sorbitan or Isosorbide.
   17. The pharmaceutical formulation of any one of embodiments 1-14, wherein the Polysorbate 20 degradation product is Lauric acid.
   18. The pharmaceutical formulation of any one of embodiments 1-17, wherein the presence of a Polysorbate 20 degradation product does not increase by more than 50% of the starting concentration of the degradation product after storage.
   19. The pharmaceutical formulation of any one of embodiments 1-17, wherein the presence of a Polysorbate 20 degradation product does not increase by more than 25% of the starting concentration of the degradation product after storage.
   20. The pharmaceutical formulation of any one of embodiments 1-17, wherein the presence of a Polysorbate 20 degradation product does not increase by more than 10% of the starting concentration of the degradation product after storage.
   21. The pharmaceutical formulation of any one of embodiments 1-20, wherein the concentration of polyester species of Polysorbate 20 remain within 10% or 20% of the starting concentration of the polyester species after storage for a period of time of at least 1 day.
   22. The pharmaceutical formulation of any one of embodiments 1-21, wherein the concentration of monoester species of Polysorbate 20 remain within 10% or 20% of the starting concentration of the monoester species after storage for a period of time of at least 1 day.
   23. The pharmaceutical formulation of any one of embodiments 1-22, wherein storage for a period of time of at least 1 day is storage for 1, 3 or 7 days.
   24. The pharmaceutical formulation of any one of embodiments 1-22, wherein storage for a period of time of at least 1 day is storage for about or more than 14 or 28 days.
   25. The pharmaceutical formulation of any one of embodiments 1-22, wherein storage for a period of time of at least 1 days is storge for about or more than 8 weeks or 12 weeks.
   26. The pharmaceutical formulation of any one of embodiments 1-22, wherein storage for a period of time of at least 1 days is storage for about or more than 1 year.
   27. The pharmaceutical formulation of any one of embodiments 1-26, wherein the storage is at 2-8° C.
   28. The pharmaceutical formulation of any one of embodiments 1-26, wherein the storage is at 25° C.
   29. The pharmaceutical formulation of any one of embodiments 1-26, wherein the storage is at 40° C.
   30. The pharmaceutical formulation of any one of embodiments 1-29, wherein the antibody has been purified from cell culture by Protein A affinity chromatography.
   31. A method of purifying protein having an Fc region from cell culture, the method comprising:
(i) loading cell culture comprising the antibody on to a Protein A chromatography column,
(ii) washing the column one, two, three or more times using one or more wash buffers, wherein at least one wash buffer comprises a pH of greater than 9,
(iii) eluting the antibody from the column,
(iv) subjecting the eluate from step (iii), comprising the antibody, to filtration and/or concentration, and
(v) formulating the antibody from step (iv) into a pharmaceutical formulation comprising adding one or more excipients, wherein the concentration of the antibody in the pharmaceutical formulation is higher than 50 g/L, and wherein the one or more excipients comprise Polysorbate 20.
   32. The method of embodiment 31, wherein the Protein A column load ratio in step (i) is 25 g/L or less.
   33. The method of embodiment 31 or 32, wherein the at least one wash buffer comprises a pH of at least or more than 10.
   34. The method of embodiment 31 or 32, wherein the at least one wash buffer comprises a pH of about 10.5.
   35. The method of any one of embodiments 31-34, wherein the at least one wash buffer comprises 1M Sodium Chloride or higher concentration of Sodium Chloride.
   36. The method of any one of embodiments 31-34, wherein the at least one wash buffer comprises concentration of Sodium Chloride that is less than 1M or less than 0.5M.
   37. The method of any one of embodiments 31-36, wherein the at least one wash buffer comprises arginine.
   38. The method of any one of embodiments 31-36, wherein the at least one wash buffer does not comprise arginine.
   39. The method of any one of embodiments 31-38, wherein the at least one wash buffer comprises sodium phosphate or sodium carbonate.
   40. The method of any one of embodiments 31-38, wherein the at least one wash buffer comprises 50 mM sodium carbonate.
   41. The method of any one of embodiments 31-40, wherein the concentration of the antibody in the pharmaceutical formulation is about or higher than 100 g/L or is about or higher than 150 g/L.
   42. The method of any one of embodiments 31-40, wherein the final concentration of the antibody in the pharmaceutical formulation is about or higher than 170 g/L.
   43. The method of any one of embodiments 31-40, wherein the final concentration of the antibody in the pharmaceutical formulation is about or higher than 200 g/L.
   44. The method of any one of embodiments 31-43, wherein the antibody is a humanized or human monoclonal antibody.
   45. The method of embodiment 44, wherein the antibody is an IgG1 antibody.
   46. The method of any one of embodiments 31-45, wherein the antibody is an anti-FcRn antibody that specifically binds to FcRn.
   47. The method of embodiment 46, wherein the anti-FcRn antibody is a fully human monoclonal IgG1 antibody.
   48. The method of embodiment 46 or 47, wherein the anti-FcRn antibody comprises (a) (i) three heavy chain CDR amino acid sequences of SEQ ID No: 27 (HCDR1), SEQ ID No: 28 (HCDR2), SEQ ID No: 29 (HCDR3); and (ii) three light chain CDR amino acid sequences of SEQ ID No: 30 (LCDR1), SEQ ID No: 31 (LCDR2), SEQ ID No: 32 (LCDR3); or
(b) (i) three heavy chain CDR amino acid sequences of SEQ ID No: 49 (HCDR1), SEQ ID No: 22 (HCDR2), SEQ ID No: 23 (HCDR3); and (ii) three light chain CDR amino acid sequences of SEQ ID No: 50 (LCDR1), SEQ ID No: 25 (LCDR2), SEQ ID No: 26 (LCDR3).
   49. The method of any one of embodiments 46-48, wherein the anti-FcRn antibody comprises (a) a heavy chain variable region amino acid sequence of SEQ ID No: 6 and a light chain variable region amino acid sequence of SEQ ID No: 16; or
(b) a heavy chain amino acid sequence of SEQ ID No: 51; and a light chain amino acid sequence of SEQ ID No: 52.
   50. The method of any one of embodiments 31-49, wherein the cell culture is harvested host cells used for production of the antibody, wherein the host cells are mammalian cells suitable for antibody production.
   51. The method of embodiment 50, wherein the mammalian cells are CHO cells.
   52. The method of any one of embodiments 31-51, wherein the step (iv) of the method comprises depth filtration
   53. The method of any one of embodiments 31-52, wherein the step (iv) of the method comprises anion exchange (AEX) chromatography.
   54. The method of any one of embodiments 31-53, wherein the step (iv) of the method comprises cation exchange (CEX) chromatography.
   55. The method of any one of embodiments 31-54, wherein the step (iv) of the method comprises viral filtration.
   56. The method of any one of embodiments 31-55, wherein the step (iv) of the method comprises ultrafiltration and/or diafiltration.
   57. The method of any one of embodiments 31-56, wherein the Polysorbate 20 in the pharmaceutical formulation is at a concentration from about 0.002% to 0.2%.
   58. The method of any one of embodiments 31-56, wherein the Polysorbate 20 in the pharmaceutical formulation is at a concentration from 0.01 to 0.05%.
   59. The method of any one of embodiments 31-56, wherein antibody is an anti-FcRn antibody that specifically binds to FcRn, and wherein the pharmaceutical formulation optionally comprises any one, two, three or more of the following: at least or about 170 g/L of the anti-FcRn antibody, 0.02% Polysorbate 20, 100 mM L-Histidine/Histidine HCl and 100 mM L-Arginine HCl in water at pH 6.0.
   60. A pharmaceutical formulation produced using the method of any one of embodiments 31-59.
   61. The pharmaceutical formulation of embodiment 60, wherein the concentration of the Polysorbate 20 remains within 30% of the starting concentration of the Polysorbate 20 after storage for a period of time of at least 1 day and/or wherein the presence of a Polysorbate 20 degradation product does not increase by more than 2 times of the starting concentration of the degradation product after storage for a period of time of at least 1 day.
   62. The pharmaceutical formulation of embodiment 61, wherein the concentration of the Polysorbate 20 remains within 20% of the starting concentration of the Polysorbate 20 after storage.
   63. The pharmaceutical formulation of embodiment 61, wherein the concentration of the Polysorbate 20 remains within 10% of the starting concentration of the Polysorbate 20 after storage.
   64. The pharmaceutical formulation of embodiment 61, wherein the concentration of the Polysorbate 20 remains within 5% of the starting concentration of the Polysorbate 20 after storage.
   65. The pharmaceutical formulation of any one of embodiments 61-64, wherein the Polysorbate 20 degradation product is a non-esterified species of Polysorbate 20.
   66. The pharmaceutical formulation of embodiment 65, wherein the non-esterified species of Polysorbate 20 is Sorbitan or Isosorbide.
   67. The pharmaceutical formulation of any one of embodiments 61-64, wherein the Polysorbate 20 degradation product is Lauric acid.
   68. The pharmaceutical formulation of any one of embodiments 61-67, wherein the presence of a Polysorbate 20 degradation product does not increase by more than 50% of the starting concentration of the degradation product after storage.
   69. The pharmaceutical formulation of any one of embodiments 61-67, wherein the presence of a Polysorbate 20 degradation product does not increase by more than 25% of the starting concentration of the degradation product after storage.
   70. The pharmaceutical formulation of any one of embodiments 61-67, wherein the presence of a Polysorbate 20 degradation product does not increase by more than 10% of the starting concentration of the degradation product after storage.
   71. The pharmaceutical formulation of any one of embodiments 61-70, wherein the concentration of polyester species of Polysorbate 20 remain within 10% or 20% of the starting concentration of the polyester species after storage for a period of time of at least 1 day.
   72. The pharmaceutical formulation of any one of embodiments 61-71, wherein the concentration of monoester species of Polysorbate 20 remain within 10% or 20% of the starting concentration of the monoester species after storage for a period of time of at least 1 day.
   73. The pharmaceutical formulation of any one of embodiments 61-72, wherein storage for a period of time of at least 1 day is storage for 1, 3 or 7 days.
   74. The pharmaceutical formulation of any one of embodiments 61-72, wherein storage for a period of time of at least 1 day is storage for about or more than 14 or 28 days.
   75. The pharmaceutical formulation of any one of embodiments 61-72, wherein storage for a period of time of at least 1 days is storge for about or more than 8 weeks or 12 weeks.
   76. The pharmaceutical formulation of any one of embodiments 61-72, wherein storage for a period of time of at least 1 days is storage for about or more than 1 year.
   77. The pharmaceutical formulation of any one of embodiments 61-76, wherein the storage is at 2-8° C.
   78. The pharmaceutical formulation of any one of embodiments 61-76, wherein the storage is at 25° C.
   79. The pharmaceutical formulation of any one of embodiments 61-76, wherein the storage is at 40° C.

### ILLUSTRATIVE EMBODIMENTS II

1. A method of purifying an anti-FcRn antibody, the method comprising
(a) subjecting a solution comprising the anti-FcRn antibody to Protein A chromatography, wherein the chromatography column is washed with a buffer having a pH of greater than 9 or equal or greater than 10;
(b) subjecting the solution to filtration and/or concentration, and
(c) formulating the anti-FcRn antibody for clinical administration, wherein the formulation of the anti-FcRn antibody comprises 170 mg/mL anti-FcRn antibody in 100 mM L-Histidine/Histidine HCl, 100 mM L-Arginine HCl and 0.02% Polysorbate 20 in water at pH 6.0.
   2. The method of embodiment 1, wherein the buffer comprises a salt at a concentration of at least 1M.
   3. The method of embodiment 1 or 2, wherein the pH is at least 10.
   4. The method of any one of embodiments 1-3, wherein the pH of the buffer is 10.5
   5. The method of any one of embodiments 1-3, wherein the pH of the buffer is 11.
   6. The method of any one of embodiments 1-5, wherein the Protein A Column load ratio is 25.0 g/Lᵣₑₛᵢₙ or less.
   7. The method of any one of embodiments 1-6, wherein the wash buffer comprises at least 1M Sodium Chloride.
   8. The method of any one of embodiments 1-7, wherein the wash buffer comprises arginine.
   9. The method of any one of embodiments 1-8, further comprising subjecting the solution to viral inactivation.
   10. The method of any one of embodiments 1-9, wherein the at least one chromatography method is Anion Exchange (AEX) chromatography.
   11. The method of any one of embodiments 1-9, wherein the at least one chromatography method is Cation Exchange (CEX) Chromatography.
   12. The method of any one of embodiments 1-11, wherein the filtration is ultrafiltration and/or diafiltration.
   13. A formulation of anti-FcRn antibody produced by the method of any one of embodiments 1-12.
   14. The formulation of embodiment 13, wherein the concentration of Polysorbate 20 in the formulation remains within 25% of the starting concentration after at least 28 days of storage at 40° C.
   15. The formulation of embodiment 13, wherein the concentration of Polysorbate 20 in the formulation remains within 5% of the starting concentration after at least 8 weeks of storage at −70° C.
   16. The formulation of embodiment 13, wherein the concentration of Polysorbate 20 in the formulation remains within 1% of the starting concentration after at least 8 weeks of storage at −70° C.
   17. The formulation of embodiment 13, wherein the concentration of Polysorbate 20 in the formulation remains within 10% of the starting concentration after at least 8 weeks of storage at 2-8° C.
   18. The formulation of embodiment 13, wherein the concentration of Polysorbate 20 in the formulation remains within 110% of the starting concentration after at least 8 weeks of storage at −25° C.

### EXAMPLES

### Example 1: Wash Buffer Optimization for PS20 Stability

### Root Cause Identification of PS20 Degradation and Degradation Mechanism

The drug substance manufacture using the bioreactor is by a fed-batch cell culture process, using commercially available, animal-component-free media. An overview of the upstream unit operations is shown in FIG. 2A. The downstream process overview is shown in FIG. 2B.

The upstream process is initiated with the thawing of a cell bank vial, followed by a series of inoculum expansion steps in shake flasks and bioreactors (cell bag and stir tank) until there are sufficient cells to inoculate a production bioreactor. The production bioreactor is operated in fed-batch mode and supplemented with base, antifoam agent, and feed solutions during the production process. The production bioreactor is harvested based on the culture duration or viability. Harvest clarification is performed using centrifugation, followed by depth filtration and bioburden reduction using a sterilizing-grade filter. The harvest is collected in a storage tank and subsequently purified using a series of chromatography and filtration steps.

The downstream process utilizes Protein A chromatography to capture IMVT-1401 protein from the clarified harvest. The Protein A Affinity Chromatography step is followed by low pH viral inactivation, Anion Exchange (AEX) Chromatography using a multi-modal resin, and Cation Exchange (CEX) Chromatography. The CEX pool is filtered through a nanofilter to remove potential viruses. The viral filtrate is ultrafiltered and diafiltered (UFDF) to generate UFDF pool. The recovered UFDF pool is formulated, filtered through a sterilizing-grade filter directly into pre-sterilized containers, and stored at ≤−60° C.

Reduced levels of Polysorbate 20 were observed in a drug substance formulation upon its storage at 2-8° C. or greater temperature. Investigation revealed that the handling procedures were not the root cause of lower concentrations of PS20. The reduced PS20 levels can be attributed to PS20 degradation.

The IMVT-1401 (Batoclimab) drug substance is manufactured from a cell bank in the upstream process which produces the antibody in a production bioreactor, followed by a downstream process that purifies and formulates the final drug substance at a target concentration of 170 mg/mL in Histidine and Arginine buffer containing and 0.02% w/v Polysorbate 20 at pH 6.0, and then is stored frozen at ≤−60° C. IMVT-1401 drug product has the same formulation as the drug substance. No adjustment in formulation occurs during drug product manufacturing, which consists of drug substance thaw, pooling, sterile filtration, and fill into the container-closure, either PFS or vial, which is stored at 2 to 8° C.

### PS20 Degradation Mechanism in IMVT-1401

There are two main mechanisms of PS20 degradation: Hydrolysis and Oxidation. Hydrolysis is characterized by 1) formation of fatty acids as a major byproduct of degradation 2) Linear kinetics 3) not occurring in placebo 4) Enzymatic reaction in active (antibody, protein, and other modality) samples (see Dwivedi et al., Int. J. of Pharmaceutics 552: 422, 2018; Weber Int J Pharm X. 2023; 6: 100202). Oxidation can be characterized by 1) Formation of fatty acid esters, peroxides, aldehyde, ketones, fatty acid esters and few fatty acids 2) Non-linear kinetics 3) Relevant for placebo and active 4) Chemical reaction Weber Int J Pharm X. 2023; 6: 100202.

To confirm the PS20 degradation observation in the sample, a short-term thermal kinetic study of drug substance at accelerated temperatures 2-8° C. and 40° C. was performed (FIG. 3). Substantial PS20 degradation was observed at both temperatures. The degradation was rapid enough that no major differences could be observed between the two temperatures. Similar degradation was not observed in placebo, formulated at same excipient and PS20 concentration, minus the antibody at 170 mg/mL (FIG. 4).

### PS20 Kinetic Study as a Surrogate for Process Impurity Mapping

To identify the point of introduction of the problematic impurity in the manufacturing process, a PS20 kinetic Study was used as a surrogate method. Process mapping study was performed across key purification steps of the drug substance process. The process intermediates such as Capture Chromatography Eluate (Protein A Pool), Viral Inactivated Pool (VIN Pool), Polishing Chromatography Eluate (AEX and CEX Pool), and Ultrafiltration/Diafiltration (UFDF Pool) were spiked with freshly prepared PS20 stock solution at the same target PS20 concentration as drug substance (0.02% w/v) and incubated at 40° C. for the duration of the kinetic study.

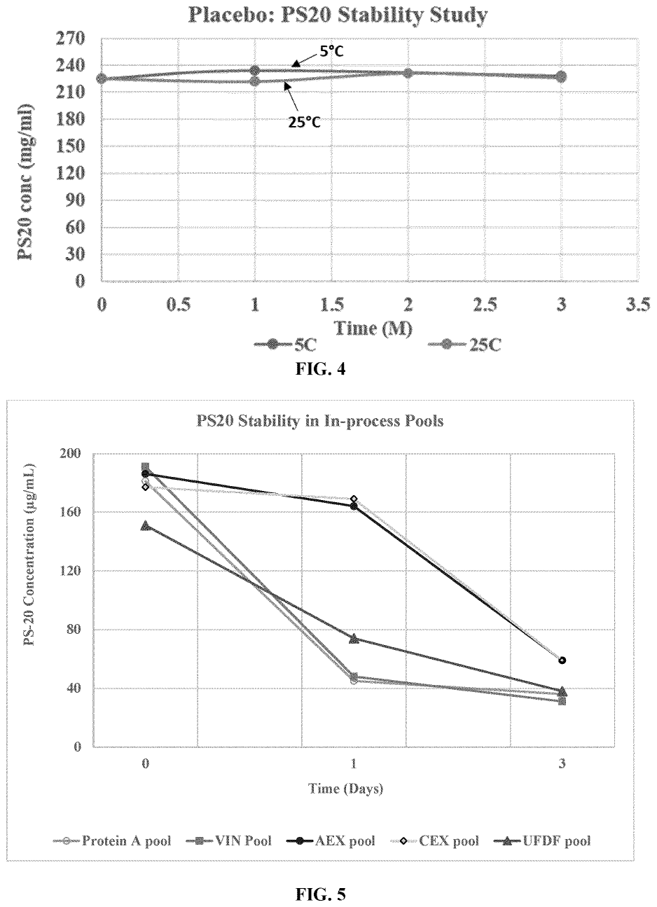

In-process pools (Protein A Chromatography, Viral Inactivation and Neutralization (VIN), Anion Exchange Chromatography (AEX) and Cation Exchange Chromatography (CEX) at various steps of the downstream purification process, were formulated at target concentration of 0.02% PS-20 and staged for short term stability study over several days at 2-8° C. (5° C.) and accelerated temperature of 40° C. As shown in FIG. 5, the PS-20 is substantially degraded over 3 days at all downstream intermediate steps. Faster degradation was observed in Protein A pool compared to AEX and CEX pools. The degradation kinetics observed in the pool from the initial drug substance downstream unit operation of Protein A Chromatography; >70% degradation of PS20 within 24 hours; indicating that the factors contributing to PS-20 degradation are potentially introduced from the drug substance upstream process and co-purify with the product during the downstream purification process.

LC-MS was performed to further characterize the PS20 degradation mechanism. It was confirmed that one of the potential mechanisms of degradation is hydrolysis due to the cleavage of the ester bond that resulted in the presence of lauric acid as a byproduct. Additionally, no significant PS20 degradation was observed in formulation buffer. Hence it was concluded that residual Host Cell Proteins (HCP) from the upstream process are the leading contributing factor for PS20 degradation of in-process and drug substance samples.

Since the HCP impurity is introduced from the upstream cell culture process and co-elutes with the product at all key purification steps, separation of the impurity from the product at the very first purification step, Protein A capture step, was evaluated.

### Protein A Affinity Chromatography Unit Process Design (Old Process)

The Protein A Affinity chromatography step serves as a capture and initial purification step. The Harvested Cell Culture Fluid (HCCF) is loaded onto a column packed with MabSelect SuRe LX resin (Cytivia Life), and is equilibrated with an equilibration buffer. The Protein A column operates in bind and elute mode, IMVT-1401 binds to the resin while the cell culture medium and host-cell impurities pass through the column. To reduce non-specific binding interactions with impurities, IMVT-1401 bound to the column is washed with intermediate wash steps (Wash 1, Wash 2 & Wash 3) prior to elution. The stringency of the wash increases from Wash 1 to Wash 2, and the column is prepared for elution by Wash 3. IMVT-1401 is eluted using a low pH elution buffer. Protein A eluate is then processed by additional downstream steps.

Unit operation flow diagram is shown in FIG. 6. As shown in FIG. 5, the Protein A Eluate Pool using this process did not result in removal of problematic impurities responsible for PS20 degradation.

The process development efforts to achieve PS20 stability at intermediate step (Protein A Eluate Pool) and eventually drug substance were conducted in a stage-wise manner.

### Wash 2 Buffer Component Screening, Screening Study to Evaluate Wash Buffer Matrix, Wash Buffer pH and Greater Wash Stringency Using Higher Concentration of Sodium Chloride (NaCl) and Addition of Arginine (One Factor at a Time, OFAT)

Representative cell culture harvest material from large scale runs was utilized as the load material. A qualified scale down model of Protein A Chromatography unit operation was utilized to execute the development work at small scale. The process was executed as described above, except the Wash 2 buffer condition, which was varied based on the experimental design.

TABLE 4
Wash 2 Buffer Conditions.

| | | | | Sodium | |
|---|---|---|---|---|---|
| | | Buffer | | Chloride | Arginine |
| Condition | Description | Matrix | pH | Conc. (M) | Conc. (M) |
| 1 | Phosphate Buffer/Low | 50 mM | 6.5 | 0.3 | 0 |
| (Control) | pH/Low Salt/No Arginine | Sodium | | | |
| (−−−) | | phosphate | | | |
| 2 | Phosphate Buffer/Low | 50 mM | 6.5 | 1 | 0 |
| | pH/High Salt/No Arginine | Sodium | | | |
| | | phosphate | | | |
| 3 | Phosphate Buffer/Low | 50 mM | 6.5 | 1 | 0.5 |
| | pH/High Salt/With Arginine | Sodium | | | |
| | | phosphate | | | |
| 4 | Phosphate | 50 mM | 7.5 | 1 | 0.5 |
| | Buffer/Intermediate | Sodium | | | |
| | pH/High Salt/With Arginine | phosphate | | | |
| 5 | Tris Buffer/High pH/High | 50 mM | 9 | 1 | 0 |
| | Salt/No Arginine | Tris | | | |
| 6 | Tris Buffer/High High | 50 mM | 10 | 1 | 0.5 |
| (+++) | pH/High Salt/With Arginine | Tris | | | |
| 7 | Tris Buffer/Intermediate | 50 mM | 7.5 | 1 | 0 |
| | pH/High Salt/No Arginine | Tris | | | |

Protein A Eluate Pool from each of the conditions was evaluated for concentration step yield, PS20 stability at 40° C. (performed by spiking PS20 at target concentration of 0.020 (w/v) and incubating at 40° C. for the duration of the kinetic study), host cell protein concentration (HCP, as shown in Table 5-Table 7), product quality attributes such as Purity by SE-HPLC, CE-SDS (Reduced/Non Reduced), and charge variants by cIEF, deamidation and oxidation by Peptide Map.

TABLE 5
Protein A Eluate Concentration, Step Yield, PS20 Stability and Host Cell Protein Concentration Test Results

| | | Protein A | Step | PS20 Content (μg/mL) | | | | | | Host Cell |
|---|---|---|---|---|---|---|---|---|---|---|
| | | Pool Conc. | Yield | | T = 6 | T = 1 | T = 3 | T = 7 | T = 14 | Protein |
| Condition | Description | g/L | % | T = 0 | hours | Day | Day | Day | Day | ng/mg |
| 1 | Phosphate | 18.7 | 98 | 223.5 | 102.3 | <100 | <100 | <100 | <100 | 1004.3 |
| | Buffer/Low pH/Low | | | | | (54) | (37) | (19) | (ND) | |
| | Salt/No Arginine | | | | | | | | | |
| 2 | Phosphate | 18.6 | 99 | 224.3 | 100.1 | <100 | NT | NT | NT | 1341.5 |
| | Buffer/Low pH/High | | | | | (52) | | | | |
| | Salt/No Arginine | | | | | | | | | |
| 3 | Phosphate | 18.7 | 100 | 226.5 | 134.8 | <100 | NT | NT | NT | 1189.8 |
| | Buffer/Low pH/High | | | | | (75) | | | | |
| | Salt/With Arginine | | | | | | | | | |
| 4 | Phosphate | 18.8 | 100 | 225.2 | 142.1 | <100 | NT | NT | NT | 1097.4 |
| | Buffer/Intermediate | | | | | (81) | | | | |
| | pH/High Salt/With | | | | | | | | | |
| | Arginine | | | | | | | | | |
| 5 | Tris Buffer/High | 22.3 | 98 | 217.8 | 132.3 | <100 | NT | NT | NT | 894.9 |
| | pH/High Salt/No | | | | | (71) | | | | |
| | Arginine | | | | | | | | | |
| 6 | Tris Buffer/High | 22.2 | 100 | 224.8 | 209.7 | 197.4 | 194 | 179 | 163 | 743.8 |
| | High pH/High | | | | | | | | | |
| | Salt/With Arginine | | | | | | | | | |
| 7 | Tris | 24.7 | 99 | 217.1 | 97 | <100 | NT | NT | NT | 1338 |
| | Buffer/Intermediate | | | | | (27) | | | | |
| | pH/High Salt/No | | | | | | | | | |
| | Arginine | | | | | | | | | |

* The Limit of Quantification (LOQ) of PS20 concentration analysis is <100 μg/mL. To check the trend of PS20 stability, LOQ results are also described in this table;
* ND: Not Detected, NT: Not Tested

TABLE 6
Protein A Eluate Product Quality Attributes Test Results

| | | | | | CE-SDS | CE-SDS | | | | |
|---|---|---|---|---|---|---|---|---|---|---|
| | | SE-HPLC (%) | | | (R) (%) | (NR) (%) | cIEF (%) | | | |
| Conditi [symbol] | Description | HMW | Monomer | LMW | HC + LC | IgG | Main pI | Basic | Main | Acidic |
| 1 | Phosphate | 1.01 | 98.76 | 0.23 | 98.47 | 95.06 | 8 | 1.58 | 60.71 | 37.71 |
| | Buffer/Low pH/Low | | | | | | | | | |
| | Salt/No Arginine | | | | | | | | | |
| 6 | Tris Buffer/High | 1.19 | 98.65 | 0.16 | 98.43 | 94.95 | 8 | 1.13 | 62.68 | 36.19 |
| | High pH/High | | | | | | | | | |
| | Salt/With Arginine | | | | | | | | | |
| 7 | Tris | 0.9 | 99.07 | 0.04 | 98.23 | 94.85 | 8 | 2.12 | 55.97 | 41.92 |
| | Buffer/Intermediate | | | | | | | | | |
| | pH/High Salt/No | | | | | | | | | |
| | Arginine | | | | | | | | | |

*Condition 2 to Condition 5 not tested for product quality, as no improvement in PS20 stability was observed [symbol]
indicates data missing or illegible when filed

TABLE 7
Protein A Eluate Deamidation and Oxidation by Peptide Map

| | | Peptide mapping (%) | | | |
|---|---|---|---|---|---|
| Condition | Description | M256 | M432 | N25 | N319 |
| 1 | Phosphate Buffer/Low pH/Low | 5 | 2.5 | 4.4 | 4.3 |
| | Salt/No Arginine | | | | |
| 6 | Tris Buffer/High pH/High Salt/ | 5 | 3.6 | 4.5 | 3.9 |
| | With Arginine | | | | |
| 7 | Tris Buffer/Intermediate pH/ | 5.8 | 3.7 | 4.5 | 4 |
| | High Salt/No Arginine | | | | |

In these experiments, the control performed like large scale process performance, hence, the scale down model was considered suitable for development work.

Wash 2 buffer condition (Condition 6) at pH 10, 1M NaCl, 0.5M Arginine in Tris matrix showed significant improvement. Thus, more than 70% of PS20 was retained during the 14 Day study, and only 27% loss observed compared to control (Condition 1) which was fully degraded within one (1) day of the stability study.

Slightly higher impurity clearance or lower HCP levels were observed in Condition 6 compared to control.

Transient exposure of high pH wash 2 buffer did not impact key product quality attributes, no impact was observed in oxidation and deamidation levels by Peptide Map.

Alkaline (High pH) of Wash 2 buffer had positive impact on PS20 stability, pH less than pH 10 had no impact.

No impact of salt at low pH on PS20 stability was observed. No impact of Arginine at low pH on PS20 stability was observed. No impact of buffer was observed at low or intermediate pH conditions (Condition 1 and Condition 7).

These data indicate that the problematic impurity responsible for rapid PS20 degradation can be removed by improving the intermediate wash (Wash 2) condition. Higher pH (pH 10 or pH>9), High Salt (1M Salt) and presence of Arginine in the wash buffer significantly improved the PS20 stability.

Wash 2 Buffer Component Screening, Design of Experiment (DoE) Study to evaluate Wash buffer pH. Sodium Chloride (NaCl) and Arginine

Representative cell culture harvest material from large scale IMVT-1401 production runs were utilized as the load material. A qualified scale down model of Protein A Chromatography unit operation (the same as above) was utilized to execute the development work at small scale. The process was executed as described above, except the Wash 2 buffer condition, which was varied based on the experimental design as described below.

A total of nine conditions were tested to understand the impact of pH, Sodium Chloride concentration, Arginine concentration and any interaction between these factors. Sodium Phosphate buffer was not evaluated as it is not a suitable matrix for high pH solutions. Condition 6 (described in Table 6 and Table 7 above) was evaluated as a control using Tris buffer matrix. However, for all other conditions, Carbonate buffer was evaluated as a buffer matrix due to better buffering capacity at higher pH. As no impact on PS20 stability at pH<10 was observed, pH≥10 was used in subsequent experiments. Two levels of Sodium Chloride were evaluated, 0.3 M (which is used in the old process) and 1M. Two levels of Arginine concentration were evaluated, no Arginine and 0.5 M concentration. Experimental conditions are described in detail in Table 8.

TABLE 8
Wash 2 Buffer Conditions

| | | Wash 2 Buffer Composition | | | |
|---|---|---|---|---|---|
| | | | | Sodium | |
| | | | | Chloride | Arginine |
| Condition | Description | Buffer Matrix | pH | Conc. (M) | Conc. (M) |
| 1 | Carbonate/pH 10/High | 50 mM Sodium | 10 | 1 | 0 |
| | Salt/No Arginine | Carbonate | | | |
| 2 (High- | Carbonate/pH 11/High | 50 mM Sodium | 11 | 1 | 0.5 |
| High-High) | Salt/With Arginine | Carbonate | | | |
| 3 (Control in | Carbonate/pH 10/High | 50 mM Sodium | 10 | 1 | 0.5 |
| Carbonate) | Salt/With Arginine | Carbonate | | | |
| 4 | Carbonate/pH 11/Low | 50 mM Sodium | 11 | 0.3 | 0 |
| | Salt/No Arginine | Carbonate | | | |
| 5 | Carbonate/pH 11/Low | 50 mM Sodium | 11 | 0.3 | 0.5 |
| | Salt/With Arginine | Carbonate | | | |
| 6 (Low- | Carbonate/pH 10/Low | 50 mM Sodium | 10 | 0.3 | 0 |
| Low-Low) | Salt/No Arginine | Carbonate | | | |
| 7 | Carbonate/pH 11/High | 50 mM Sodium | 11 | 1 | 0 |
| | Salt/No Arginine | Carbonate | | | |
| 8 | Carbonate/pH 10/Low | 50 mM Sodium | 10 | 0.3 | 0.5 |
| | Salt/With Arginine | Carbonate | | | |
| 9 (Control) | Carbonate/pH 10/High | 50 mM Tris | 10 | 1 | 0.5 |
| | Salt/With Arginine | | | | |

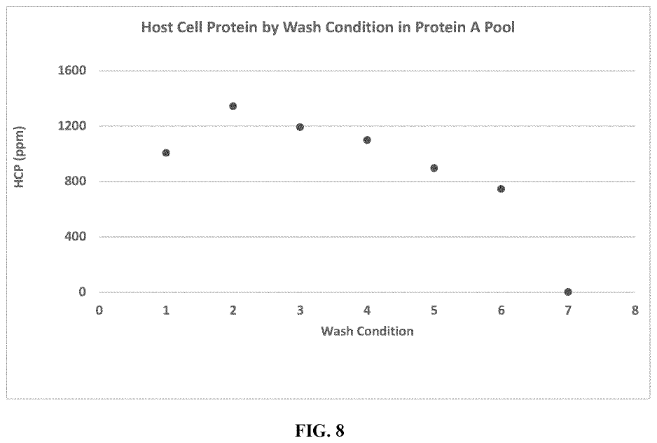

The Protein A eluate pool from each of the conditions was evaluated for concentration step yield, PS20 stability at 40° C. (performed by spiking PS20 at target concentration of 0.02% (w/v) and incubating at 40° C. for the duration of the kinetic study), HCP concentration (as shown in Table 9 and FIG. 8), and product quality attributes (as shown in Table 10).

TABLE 9
Protein A Eluate Concentration, Step Yield, PS20 Stability and Host Cell Protein Concentration Test Results

| | | Protein A | | | | | | | | | | |
|---|---|---|---|---|---|---|---|---|---|---|---|---|
| | | Eluate | Step | PS20 Content (μg/mL) | | | | | | | | |
| | | Pool Conc. | Yield | | 1 | 3 | 7 | 10 | 14 | 21 | 30 | HCP |
| Condition | Description | g/L | % | 0 | Day | Day | Day | Day | Day | Day | Day | ng/mg |
| 1 | Carbonate/pH | 18.2 | 99 | 199.1 | 181.9 | 176.7 | 178 | 178 | 174 | 192 | 176 | 871.7 |
| | 10/High | | | | | | | | | | | |
| | Salt/ No | | | | | | | | | | | |
| | Arginine | | | | | | | | | | | |
| 2 | Carbonate/pH | 18.2 | 97 | 202.9 | 169.9 | NA | 179 | 183 | 174 | 197 | 177 | 486.3 |
| (High- | 11/High | | | | | | | | | | | |
| High- | Salt/With | | | | | | | | | | | |
| High) | Arginine | | | | | | | | | | | |
| 3 | Carbonate/pH | 18.7 | 103 | 197.8 | 180 | NA | 173 | 172 | 172 | 178 | 172 | 1020.7 |
| (Control in | 10/High | | | | | | | | | | | |
| Carbonate) | Salt/With | | | | | | | | | | | |
| | Arginine | | | | | | | | | | | |
| 4 | Carbonate/pH | 18.3 | 97 | 215.1 | 173.7 | 189.7 | 177 | 183 | 181 | 181 | 180 | 536.1 |
| | 11/Low | | | | | | | | | | | |
| | Salt/No | | | | | | | | | | | |
| | Arginine | | | | | | | | | | | |
| 5 | Carbonate/pH | 20.2 | 90 | 220.6 | 170.5 | 170.8 | 173 | 177 | 183 | 187 | 184 | 446.6 |
| | 11/Low | | | | | | | | | | | |
| | Salt/With | | | | | | | | | | | |
| | Arginine | | | | | | | | | | | |
| 6 | Carbonate/pH | 22.1 | 100 | 213.6 | 165.4 | 161.5 | 161 | 147 | 162 | 165 | 154 | 659 |
| (Low- | 10/Low | | | | | | | | | | | |
| Low- | Salt/No | | | | | | | | | | | |
| Low) | Arginine | | | | | | | | | | | |
| 7 | Carbonate/pH | 22.1 | 98 | 226.1 | 171.6 | 162.5 | 180 | 176 | 178 | 174 | 183 | 555.2 |
| | 11/High | | | | | | | | | | | |
| | Salt/No | | | | | | | | | | | |
| | Arginine | | | | | | | | | | | |
| 8 | Carbonate/pH | 22.2 | 103 | 221.7 | 166.2 | 148.9 | 166 | 162 | 162 | 163 | 156 | 871.2 |
| | 10/Low | | | | | | | | | | | |
| | Salt/With | | | | | | | | | | | |
| | Arginine | | | | | | | | | | | |
| 9 | Carbonate/pH | 18.9 | 104 | 220.1 | 172.8 | 168.2 | 173 | 173 | 166 | 190 | 161 | 1174.6 |
| (Control) | 10/High | | | | | | | | | | | |
| | Salt/With | | | | | | | | | | | |
| | Arginine | | | | | | | | | | | |

TABLE 10
Protein A Eluate Product Quality Attributes Test Results

| | | | | | CE-SDS | CE-SDS | | | | |
|---|---|---|---|---|---|---|---|---|---|---|
| | | SE-HPLC (%) | | | (R) (%) | (NR) (%) | cIEF (%) | | | |
| Condition | Description | HMW | Monomer | LMW | HC + LC | IgG | Main pI | Basic | Main | Acidic |
| 1 | Carbonate/pH | 1.65 | 98.18 | 0.18 | 97.75 | 95.76 | 7.9 | 1.38 | 61.77 | 36.85 |
| | 10/High | | | | | | | | | |
| | Salt/No | | | | | | | | | |
| | Arginine | | | | | | | | | |
| 2 | Carbonate/pH | 2.58 | 97.24 | 0.18 | 97.6 | 95.07 | 7.9 | 1.46 | 60.86 | 37.69 |
| (High- | 11/ High | | | | | | | | | |
| High- | Salt/With | | | | | | | | | |
| High) | Arginine | | | | | | | | | |
| 3 | Carbonate/pH | 1.05 | 98.8 | 0.15 | 97.77 | 95.24 | 7.9 | 1.49 | 60.45 | 38.06 |
| (Control in | 10/High | | | | | | | | | |
| Carbonate) | Salt/With | | | | | | | | | |
| | Arginine | | | | | | | | | |
| 4 | Carbonate/pH | 2.19 | 97.75 | 0.06 | 97.69 | 95.4 | 7.9 | 1.39 | 59.64 | 38.97 |
| | 11/Low | | | | | | | | | |
| | Salt/No | | | | | | | | | |
| | Arginine | | | | | | | | | |
| 5 | Carbonate/pH | 6.26 | 93.6 | 0.14 | 97.59 | 94.85 | 7.9 | 1.44 | 60.46 | 38.1 |
| | 11/Low | | | | | | | | | |
| | Salt/With | | | | | | | | | |
| | Arginine | | | | | | | | | |
| 6 | Carbonate/pH | 1.54 | 98.3 | 0.15 | 97.55 | 95.47 | 7.9 | 1.48 | 60.85 | 37.67 |
| (Low- | 10/Low | | | | | | | | | |
| Low- | Salt/No | | | | | | | | | |
| Low) | Arginine | | | | | | | | | |
| 7 | Carbonate/pH | 2.12 | 97.75 | 0.12 | 97.48 | 95.24 | 7.9 | 1.38 | 58.57 | 40.05 |
| | 11/High | | | | | | | | | |
| | Salt/No | | | | | | | | | |
| | Arginine | | | | | | | | | |
| 8 | Carbonate/pH | 1.44 | 98.4 | 0.16 | 97.69 | 95.35 | 7.9 | 1.47 | 59.11 | 39.42 |
| | 10/Low | | | | | | | | | |
| | Salt/With | | | | | | | | | |
| | Arginine | | | | | | | | | |
| 9 | Carbonate/pH | 1.46 | 98.36 | 0.18 | 97.51 | 95.05 | 7.9 | 1.54 | 58.07 | 40.39 |
| (Control) | 10/High | | | | | | | | | |
| | Salt/With | | | | | | | | | |
| | Arginine | | | | | | | | | |

TABLE 11
Protein A Eluate Deamidation and Oxidation by Peptide Map

| | | Peptide mapping (%) | | | |
|---|---|---|---|---|---|
| Condition | Description | M256 | M432 | N25 | N319 |
| 1 | Carbonate/pH 10/High | 4.6 | 2.6 | 3.1 | 2.9 |
| | Salt/No Arginine | | | | |
| 2 (High-High- | Carbonate/pH 11/High | 4 | 2.8 | 3.5 | 2.9 |
| High) | Salt/With Arginine | | | | |
| 3 (Control in | Carbonate/pH 10/High | 5.1 | 3.6 | 3.2 | 3 |
| Carbonate) | Salt/With Arginine | | | | |
| 4 | Carbonate/pH 11/Low | 4.7 | 2.8 | 3.5 | 3.2 |
| | Salt/No Arginine | | | | |
| 5 | Carbonate/pH 11/Low | 3.8 | 2.5 | 3.4 | 3.2 |
| | Salt/With Arginine | | | | |
| 6 (Low-Low- | Carbonate/pH 10/Low | 5 | 3 | 3.4 | 3.2 |
| Low) | Salt/No Arginine | | | | |
| 7 | Carbonate/pH 11/High | 5.1 | 3 | 3.5 | 3.1 |
| | Salt/No Arginine | | | | |
| 8 | Carbonate/pH 10/Low | 5.5 | 3.9 | 3 | 3.1 |
| | Salt/With Arginine | | | | |
| 9 (Control) | Carbonate/pH 10/High | 6.7 | 4.4 | 3.3 | 3.2 |
| | Salt/With Arginine | | | | |

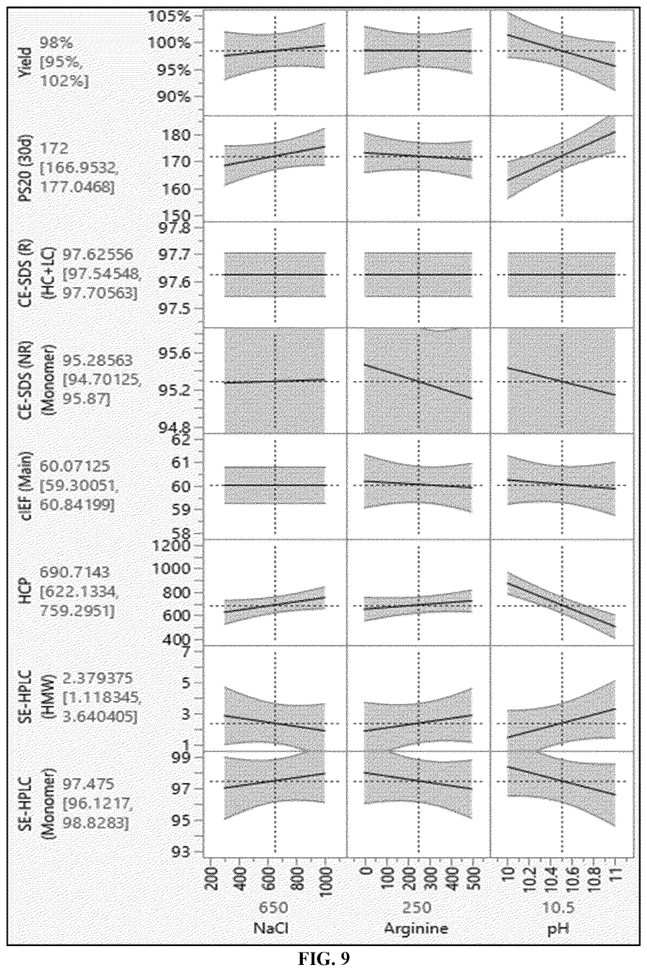

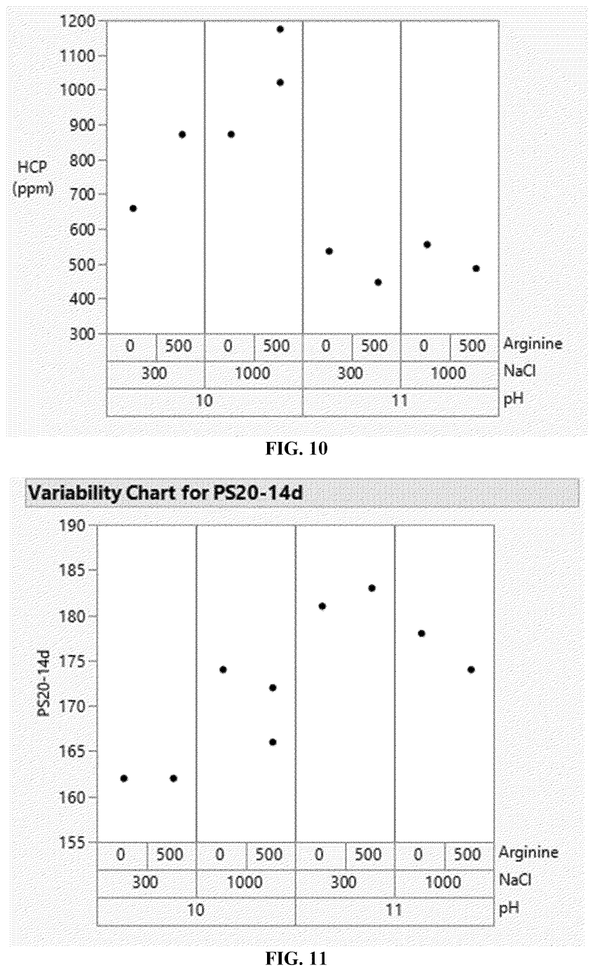

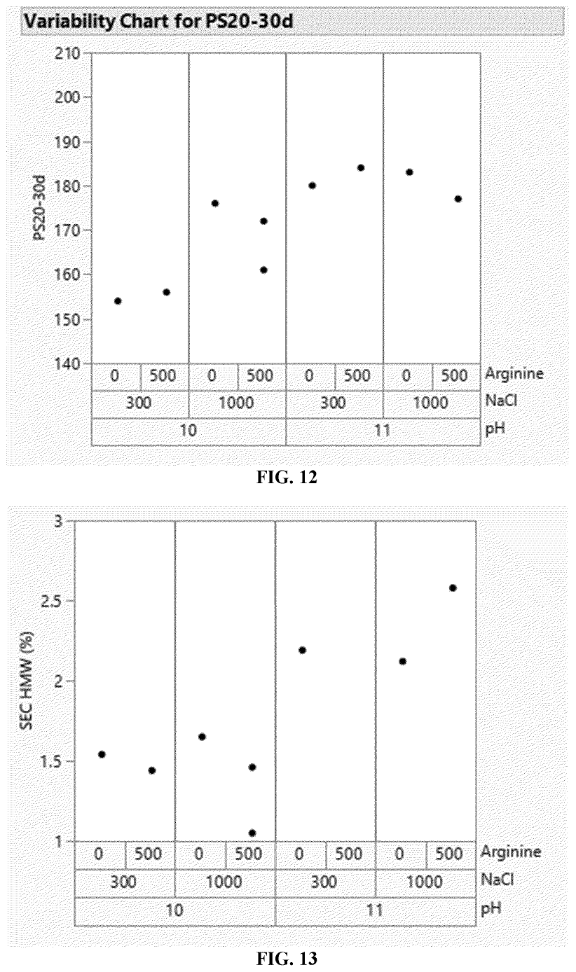

A prediction profiler of each factor is derived through statistical analysis using JIMP software, shown in FIG. 9. The pH of the wash 2 buffer showed a strong positive correlation with PS20 stability at day 14 (FIG. 11) and day 30 (FIG. 12). Sodium chloride concentration showed a weak positive correlation and Arginine concentration showed no correlation with PS20 stability.

These results suggest that the main contributing factor for PS20 stability is the wash 2 buffer pH. All conditions showed significantly improved PS20 stability, however, compared to pH 10 wash buffer, pH 11 wash conditions showed more stable PS20 profile (FIGS. 11 and 12). The PS20 stability study was performed at stressed temperature of 40° C., hence, beyond Day 14, a further reduction of PS20 was expected due to potential oxidation. However, results obtained on day 14 and day 30 show similar overall trends. Additionally, a negative correlation of HCP with buffer pH indicates that high pH condition of wash 2 buffer effectively removes the problematic host cell proteins and enzymes derived from the host cell line or upstream bioreactor production process which may be responsible for PS20 degradation.

Process step yield showed a negative correlation with pH, suggesting that IMVT-1401 may be disassociating from the column due to high pH of the wash buffer. No significant difference in product quality in terms of intact IgG, charge variants, deamidation of Asparagine (N25 and N319) and/or oxidation of Methionine (M256 and M432) was observed at any of the tested conditions, indicating the high pH has no impact on product purity. However, in terms of impurity levels, some differences were noted by SE-HPLC. Thus, higher HMW levels were observed at pH 11 when compared to pH 10 (see Table 11, condition 2).

Hence, it is concluded that high pH is the dominant factor to achieve PS20 stability. Lower HCP levels at high pH were observed based on general HCP test kit. Total lower HCP may mean lower HCP heterogeneity. Higher Sodium Chloride concentration (1 M) had positive impact on HCP removal at lower pH, however, at high pH, no impact of Sodium Chloride was observed. Arginine addition did not correlate with greater HCP removal, irrespective of the pH and salt concentration.

Design of Experiment (DoE) Study to Integrate Optimized Wash 2 Buffer with Additional Protein A Chromatography Operational Parameters

It was concluded based on the experiments described above that pH and pH-Sodium Chloride interaction are the main effects impacting PS20 stability. The wash 2 buffer at pH 10 to pH 11 and 1 M Sodium Chloride concentration were effective in achieving PS20 stability, it appears that higher the pH, more stable was the PS20 profile during the kinetic study.

Sodium Chloride concentration remained consistent at 1M and the buffer matrix was fixed as Carbonate. The pH range from pH 10 to pH 11 was further evaluated to find the optimal operating range. Additionally, the chromatography parameters IMVT-1401 load ratio (g/Lᵣₑₛᵢₙ), wash 2 buffer volume, wash 3 buffer pH, and elution buffer pH were evaluated in a multivariate DoE study. Representative cell culture harvest material from large scale IMVT-1401 production runs was utilized as the load material. A qualified scale down model of Protein A chromatography unit operation was utilized to execute the DoE at small scale. The process was executed as described above, except the factors which were varied based on the experimental design as described below.

### Operating Parameter Design Space Characterization

A central composite, face centered, 3-level design was utilized for the Protein A multivariate DoE experiment. The thirty-two run design was executed to evaluate the five parameters and the characterization as shown in Table 12. Detailed design is shown in Table 13.

TABLE 12
Protein A Process Chromatography Operating Parameter
Design Space. Parameters and Characterization Range.

| Parameter | Characterized Range |
|---|---|
| Load Ratio (g/Lᵣₑₛᵢₙ) | 20-40 |
| Wash2 Buffer pH | 10.0-11.0 |
| Wash2 Buffer Volume (Column Volume) | 4-6 |
| Wash3 Buffer pH | 5.6-6.0 |
| Elution Buffer pH | 3.2-3.6 |

TABLE 13
Protein A Chromatography Operating Parameter
Design Space Characterization Design

| | | DoE parameters | | | | |
|---|---|---|---|---|---|---|
| | | Load | Wash2 | Wash2 | Wash3 | Elution |
| | | Ratio | pH | Vol. | pH | pH |
| Run No. | Pattern | g/L resin | pH | CV | pH | pH |
| 1 | −++−− | 20 | 11.0 | 6 | 5.6 | 3.2 |
| 2 | +−+−− | 40 | 10.0 | 6 | 5.6 | 3.2 |
| 3 | 00000 | 30 | 10.5 | 5 | 5.8 | 3.4 |
| 4 | −−−−− | 20 | 10.0 | 4 | 5.6 | 3.2 |
| 5 | ++++− | 40 | 11.0 | 6 | 6.0 | 3.2 |
| 6 | −−+−+ | 20 | 10.0 | 6 | 5.6 | 3.6 |
| 7 | +−−−+ | 40 | 10.0 | 4 | 5.6 | 3.6 |
| 8 | 00A00 | 30 | 10.5 | 6 | 5.8 | 3.4 |
| 9 | ++−−− | 40 | 11.0 | 4 | 5.6 | 3.2 |
| 10 | 00000 | 30 | 10.5 | 5 | 5.8 | 3.4 |
| 11 | A0000 | 40 | 10.5 | 5 | 5.8 | 3.4 |
| 12 | 00a00 | 30 | 10.5 | 4 | 5.8 | 3.4 |
| 13 | −−−++ | 20 | 10.0 | 4 | 6.0 | 3.6 |
| 14 | −−++− | 20 | 10.0 | 6 | 6.0 | 3.2 |
| 15 | 00000 | 30 | 10.5 | 5 | 5.8 | 3.4 |
| 16 | −++++ | 20 | 11.0 | 6 | 6.0 | 3.6 |
| 17 | a0000 | 20 | 10.5 | 5 | 5.8 | 3.4 |
| 18 | 00000 | 30 | 10.5 | 5 | 5.8 | 3.4 |
| 19 | +−+++ | 40 | 10.0 | 6 | 6.0 | 3.6 |
| 20 | 0000A | 30 | 10.5 | 5 | 5.8 | 3.6 |
| 21 | ++−++ | 40 | 11.0 | 4 | 6.0 | 3.6 |
| 22 | −+−−+ | 20 | 11.0 | 4 | 5.6 | 3.6 |
| 23 | +++−+ | 40 | 11.0 | 6 | 5.6 | 3.6 |
| 24 | 000A0 | 30 | 10.5 | 5 | 6.0 | 3.4 |
| 25 | 000a0 | 30 | 10.5 | 5 | 5.6 | 3.4 |
| 26 | −+−+− | 20 | 11.0 | 4 | 6.0 | 3.2 |
| 27 | 00000 | 30 | 10.5 | 5 | 5.8 | 3.4 |
| 28 | 0a000 | 30 | 10.0 | 5 | 5.8 | 3.4 |
| 29 | 0000a | 30 | 10.5 | 5 | 5.8 | 3.2 |
| 30 | +−−+− | 40 | 10.0 | 4 | 6.0 | 3.2 |
| 31 | 00000 | 30 | 10.5 | 5 | 5.8 | 3.4 |
| 32 | 0A000 | 30 | 11.0 | 5 | 5.8 | 3.4 |

### Protein A Chromatography Operating Parameter Design Space Characterization.

### Study Result, Discussion and Conclusion

Protein A eluate was tested for several product quality attributes and PS20 stability study. The criterion for evaluation was determined based on previous process experience to evaluate practical impact on product quality attributes.

A prediction profiler of each factor was derived through statistical analysis using JMP software, shown in FIG. 9. The pH of the wash 2 buffer showed a strong positive correlation with PS20 stability (PS20-14d; FIG. 11). An additional factor, load ratio, was identified as a contribution factor and had a negative co-relation with PS20 stability, as shown in FIG. 16.

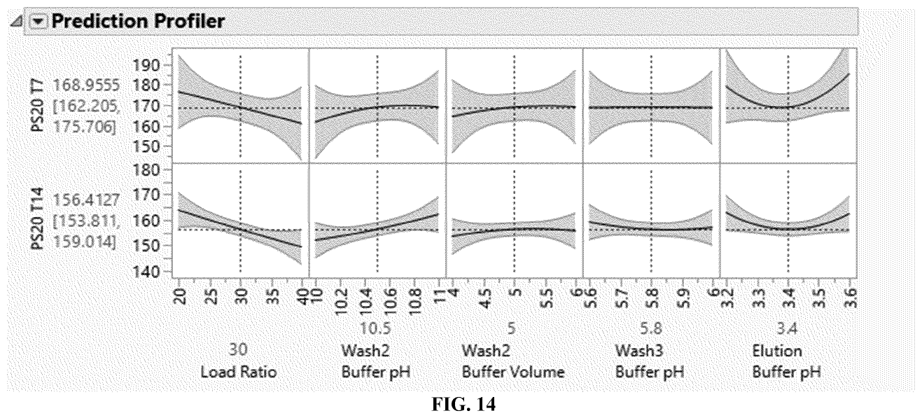

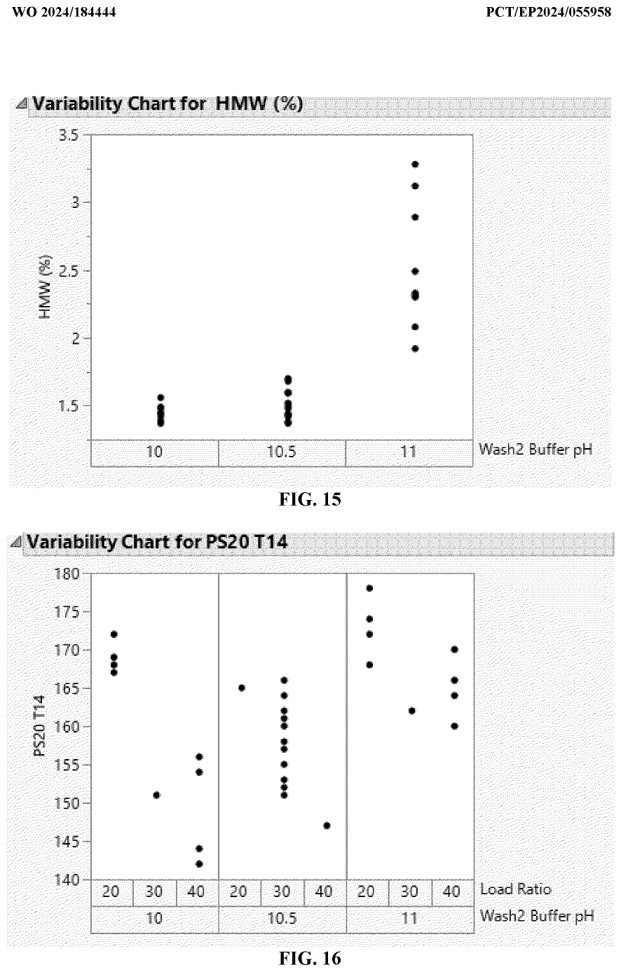

These results suggest that the main contributing factor for PS20 stability is wash 2 buffer pH and the column loading ratio. The high pH also impacted the HMW levels, however, no significant increase in HMW was observed up to pH 10.50 (FIG. 13). Additionally, a negative correlation of HCP with buffer pH and loading ratio (FIG. 14) indicates that high pH condition of wash 2 buffer and lower loading ratio effectively removes the problematic host cell proteins and enzymes derived from the host cell line or upstream bioreactor production process which may be responsible for PS20 degradation. Total lower HCP may mean lower HCP heterogeneity.

Process step yield showed a negative correlation with pH, suggesting that IMVT-1401 may be disassociating from the column due to high pH of the wash buffer. No significant difference in product quality in terms of Intact IgG, charge variants, deamidation of Asparagine (N25 and N319) and oxidation of Methionine (M256 and M432) was observed at any of the conditions tested, indicating the high pH has no impact on product purity. However, in terms of impurity levels, some differences were noted by SE-HPLC. Thus, higher HMW levels were observed at high buffer pH, pH 11 when compared to pH 10.

Hence, it is concluded that High pH of Wash 2 buffer and lower loading of the protein A column are the dominant factor to achieve PS20 stability.

In next set of experiments, the new Protein A process was integrated with remaining downstream unit operation. The antibody concentration of the Protein A eluate is approximately 15 g/L, and final DS concentration of IMVT-1401 is 170 g/L. Hence, as the molecule moves further along the downstream purification process and is eventually concentrated and formulated in final formulation buffer using Ultra Filtration/Diafiltration (UFDF), the antibody is concentrated more than 10-fold to achieve the final Bulk Drug Substance (BDS) concentration of 170 g/L. It is possible that some problematic impurities also concentrate along with the antibody and/or are enriched in the process. It is important to demonstrate sufficient reduction of these unintended, problematic HCPs at the Protein A stage itself, and assess any further potential reduction or concentration of these HCP as further downstream processing is performed.

The viral inactivation (VI) step achieves inactivation of potential adventitious viruses by low pH treatment.

The combined Protein A eluate pool from all cycles was subjected to viral inactivation, which was performed immediately after the collection of the eluate of the last Protein A chromatography cycle.

The eluate was further subjected to Anion exchange (AEX) chromatography to remove impurities, such as size variants, HCP, residual Protein A, and HCD. Subsequently, cation exchange (CEX) chromatography was applied to remove impurities, such as size variants, HCP, residual Protein A, and HCD.

The CEX eluate was filtered through a 0.2 μm filter.

Viral filtration (VF) was used to sterically remove both enveloped and non-enveloped viruses that may be present in the CEX pool.

Lastly, Ultrafiltration/Diafiltration (UFDF) was performed to exchange the buffer to the diafiltration buffer and to concentrate the IMVT-1401 product to the target concentration. The UFDF pool was diluted to a target protein concentration using DF buffer (100 mM Histidine, 100 mM Arginine, pH 6.0) before forward processing to bulk drug substance formulation.

The purpose of the formulation, final filtration, and fill unit operation is to add excipients, perform final concentration adjustment, remove potential bioburden from the final formulated DS, and transfer the product into bulk storage containers. The final composition of the DS is 170 mg/mL IMVT-1401, 100 mM histidine and 100 mM arginine with 0.02% (w/v) polysorbate 20.

The host cell proteins (HCP) was significantly reduced throughout the downstream process. Not all host cell proteins are considered problematic for PS20 stability. As shown in Table 14, improved wash 2 condition at Protein A stage did not negatively impact yield (100% yield was observed). Additionally, as observed previously, slightly high HMW levels (1.59%) were observed at the Protein A eluate stage, however, HMW levels were reduced through the downstream process and no impact on HMW levels was observed between old and new process at the BDS stage. The product quality in terms of purity (CESDS (R/NR) and charge variants (cIEF), as shown in Table 14, also showed no impact of the process changes.

TABLE 14
Protein Concentration, Step Yield, Purity (SEC, CE-SDS R/NR), charge variants
by cIEF, Host Cell Protein (HCP) and Host Cell DNA (HCD) Test Results

| | | | | | | | | | | CE-SDS | |
|---|---|---|---|---|---|---|---|---|---|---|---|
| | | | | | | cIEF (% Area) | | | | (NR, % Area) | |
| Sample | Concentration | Yield | SEC (% Area) | | | | | | Main | Intact | |
| Description | (g/L) | (%) | HMW | Monomer | LMW | Basic | Main | Acidic | pI | IgG | Unknown |
| PA load | 2.7 | | 1.00 | 98.98 | 0.03 | | | | | | |
| PA Pool | 16.7 | 100 | 1.58 | 98.34 | 0.09 | 1.26 | 66.01 | 32.73 | 8.0 | 95.75 | 1.91 |
| VIN Pool | 11.7 | 98 | 1.01 | 98.89 | 0.10 | NT | NT | NT | NT | NT | NT |
| AEX Pool | 10.2 | 94 | 0.72 | 99.23 | 0.06 | NT | NT | NT | NT | NT | NT |
| CEX Pool | 21.8 | 101 | 0.94 | 99.01 | 0.05 | NT | NT | NT | NT | NT | NT |
| VF Pool | 9.5 | 98 | 0.95 | 99.00 | 0.05 | NT | NT | NT | NT | NT | NT |
| UFDF Pool | 177.5 | 89 | 0.96 | 99.00 | 0.05 | NT | NT | NT | NT | NT | NT |
| BDS | 174 | NA | 0.94 | 99.01 | 0.05 | 2.23 | 64.66 | 33.12 | 7.9 | 96.04 | 1.88 |

| | CE-SDS (R, % Area) | | | | | |
|---|---|---|---|---|---|---|
| Sample | Unknown | Non | Purity | Unknown | HCP | HCD |
| Description | (Fragments) | Glycosylated | (HC + LC) | (Post-HC) | (ng/mg) | (pg/mg) |
| PA load | | | | | | |
| PA Pool | 0.12 | 0.37 | 97.92 | 1.59 | 1182 | 888.9 |
| VIN Pool | NT | NT | NT | NT | 33 | <0.51 |
| AEX Pool | NT | NT | NT | NT | 8 | <0.28 |
| CEX Pool | NT | NT | NT | NT | 5 | <0.59 |
| VF Pool | NT | NT | NT | NT | NT | NT |
| UFDF Pool | NT | NT | NT | NT | NT | NT |
| BDS | 0.15 | 0.37 | 97.86 | 1.63 | <0.14 | <0.14 |

The HCP values shown above are total HCP concentrations per microgram of protein. The HCP test kit is a generic kit for CHO cell expression system and not specifically designed for the IMVT-1401 expression cell line. Hence, it is possible that removal of the problematic host cell proteins that led to the improved PS20 stability may not be detected by the current HCP kit. Therefore, PS20 kinetic study was used as a surrogate for measurement of the PS20 stability and absence of problematic host cell proteins.

PS20 stability was evaluated at accelerated temperature of 40° C. for Protein A eluate, VIN/Depth filtered Pool, AEX Pool, CEX Pool, UF1 pool and UFDF Pool. Additionally, the UFDF pool was evaluated for PS20 stability at 2-8-degree C. PS20 was added to these intermediates at target concentration of 200 μg/mL, samples were held at the study temperature for the duration of the study and evaluated at the specific timepoints per the study. Results of the PS20 kinetic study are shown in Table 15. As shown, by day 14 the Protein A eluate PS20 levels decreased by 13% and by day 28 the levels had overall decreased by 22% compared to day 0. For the UFDF pool, at 40° C., the PS20 levels decreased by 11% by day 14 and remained stable at later timepoints. Overall, stable PS20 levels were observed at 2-8° C. and accelerated temperature of 40° C.

Overall variability of the PS20 method is expected to be approximately 20%. Therefore, the loss of PS20 was at the Protein A eluate stage, subsequent intermediate steps and upon concentration at UFDF step was within the expected variability. Hence, the effectiveness of the new Protein A process in the removal of problematic impurities is demonstrated even at high concentration of IMVT-1401 (>170 mg/mL).

TABLE 15
PS20 stability at intermediate steps at various hold temperature

| Condition | | PS20 Content (microgram/mL) | | | | | |
|---|---|---|---|---|---|---|---|
| Process | Hold | Day | Day | Day | Day | Day | Day |
| Step/Sample | Temperature | 0 | 3 | 7 | 14 | 21 | 28 |
| Protein A | 40° C. | 197 | 192 | 175 | 172 | 163 | 154 |
| UFDF Pool | 2-8° C. | 192 | 180 | 180 | 173 | 177 | 173 |
| UFDF Pool | 40° C. | 192 | 182 | 177 | 170 | 171 | 155 |

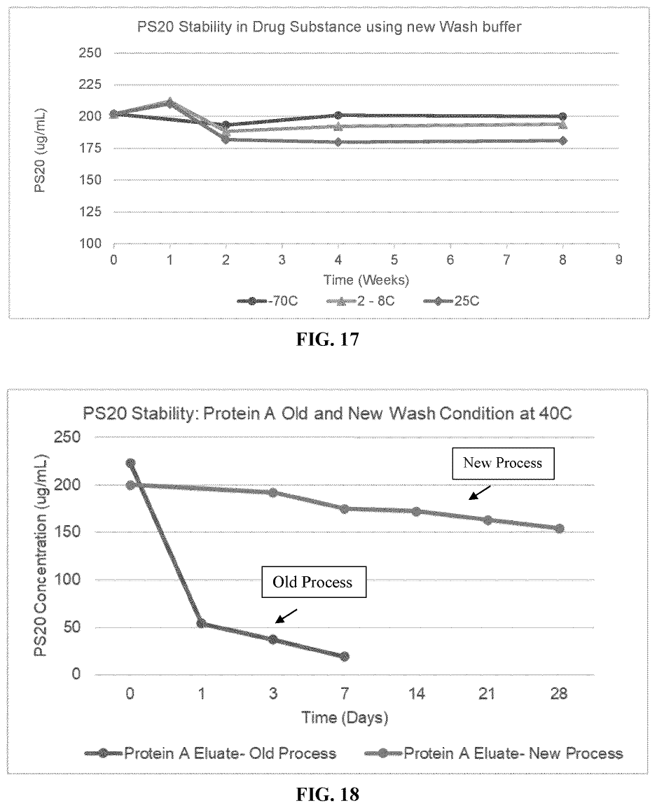

The UFDF pool was formulated at the target PS20 concentration of 200 μg/mL and filtered through a 0.2 μm filter to generate the final BDS. PS20 stability at the BDS stage was evaluated at three temperatures: storage (−70° C.), accelerated (2-8° C.), and stressed (25° C.) for 8-weeks as shown in Table 16 and FIG. 17. No loss of PS20 was observed over 8 weeks at any of the temperatures tested; the values at week 8 were within the analytical variability of the methods used.

TABLE 16
PS20 stability at BDS at various hold temperatures

| | | Weeks | | | | |
|---|---|---|---|---|---|---|
| Sample | Temperature | 0 | 1 | 2 | 4 | 8 |
| Drug substance | −70° C. | 202 | | 193 | 201 | 200 |
| | 2-8° C. | | 212 | 188 | 192 | 194 |
| | 25° C. | | 210 | 182 | 180 | 181 |

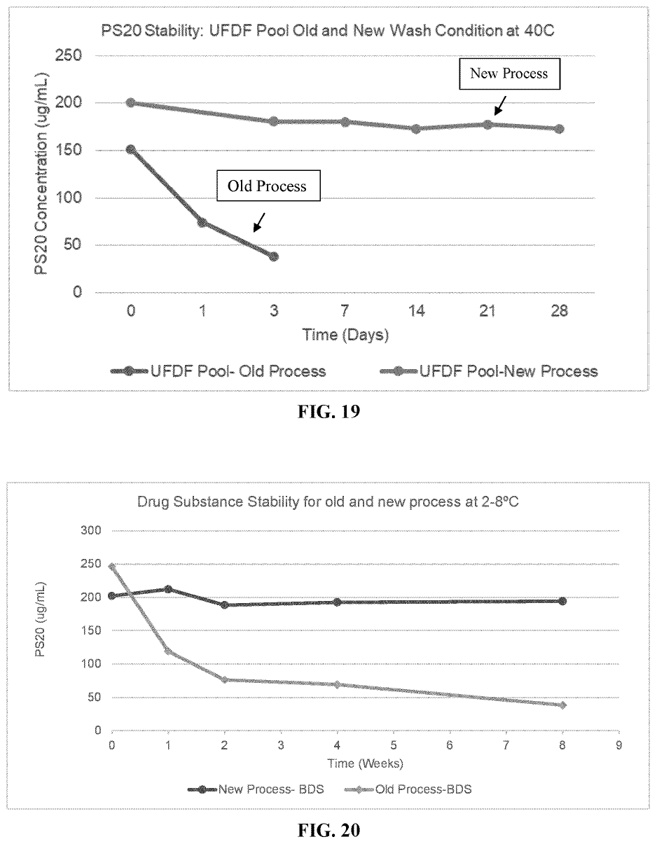

PS20 stability between the old and the new process at Protein A eluate, UFDF pool and BDS stage is shown in FIGS. 18-20. As shown in FIG. 18, Protein A eluate generated using the new process showed significant improvement in PS20 stability when compared to the old process. Thus, PS20 levels were below quantitation limit by day 1 of the kinetic study with the old process, while PS20 was retained and only 23% loss was observed by Day 28 with the new process at highly accelerated temperature of 40° C. Similarly, at the UFDF pool stage, PS20 was below quantitation limit by Day 1 for the old process, however, no substantial loss of PS20 was observed at highly accelerated temperature after 28 days in the UFDF pool generated using the new optimized Protein A condition (FIG. 19). Additionally, PS20 stability was observed for 8 weeks at accelerated temperatures in the final drug substance (FIG. 20).

The possible chemical modifications of IMVT-1401 (deamidation, oxidation, pyro-glutamation, N-glycosylation, and glycation) were also monitored. No negative impact on chemical modification of the antibody was observed and the BDS using the new Protein A optimization was comparable to that generated using the old process.

Hence, the Protein A Chromatography changes were successfully integrated with the remaining downstream process to yield BDS of comparable quality with stable PS20 profile. These process changes were confirmed at large scale and demonstrated similar results, as discussed in section [0255].

### PS20 Profile Characterization by Liquid Chromatography/Mass Spectrometry

LC/MS was performed for further characterization of the polysorbate degradation. Polysorbate species include Polyexyethylene (POE) sorbitan and isosorbide esters with varying degree of esterification (mono, di, tri etc.) varying fatty acid chain lengths, and varying units of ethoxylation. PS20 primarily contains laurate as the fatty acid chain. As described above, there are two main degradation pathways of PS20, one is oxidative, and the other is hydrolytic. The rapid increase in the concentration of the non-esterified species (Sorbitan and Isosorbide) indicated that the degradation most likely proceeds via hydrolysis, resulting in the loss of the fatty acid tail (see Dwivedi et al., Int. J. of Pharmaceutics 552:422, 2018). Loss of the laurate fatty chain can be measured by performing a scan in negative ion mode where the expected degradation product/ion of hydrolysis Lauric Acid can be observed. A quantitative analysis of these species was performed for the old and the new process.

### PS20 Profile (Esters) and Lauric Acid (LA) Levels in Protein A Eluate

A PS20 kinetic study at 40° C. over a period of 14 days, using the methods described above, was performed for the Protein A eluate for the old and new process.

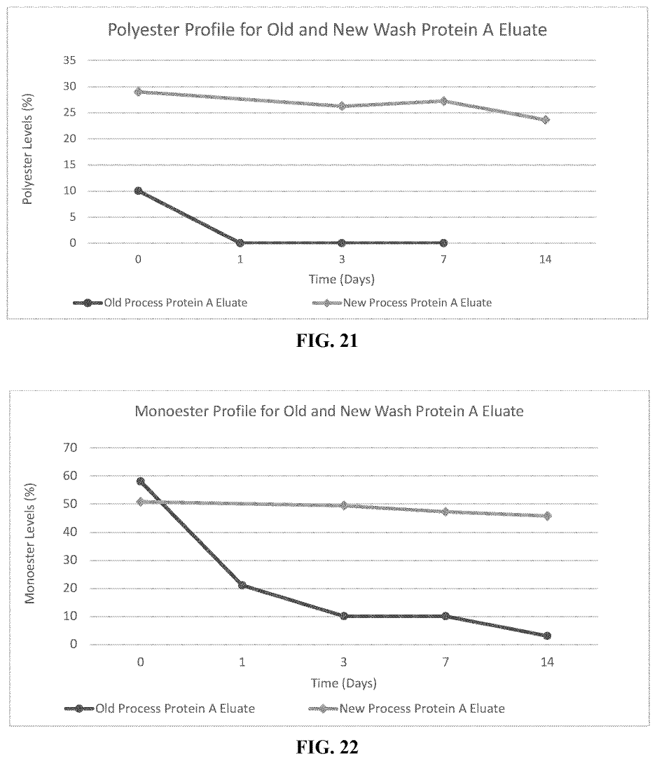

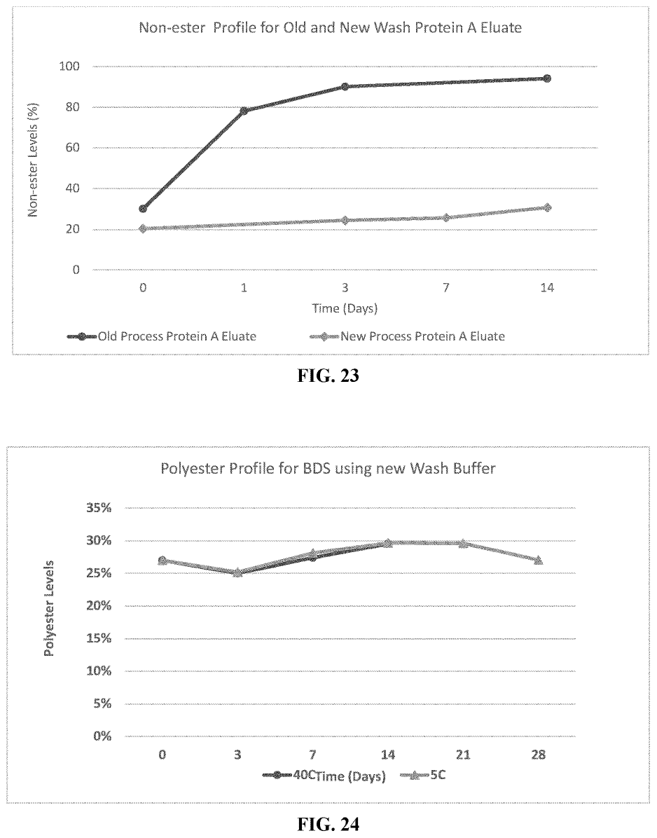

As shown below, the old process shows complete reduction of the key components of PS20, i.e. polyester (FIG. 21), and monoester (FIG. 22), and an increase in non-esterified species (FIG. 23). This suggests degradation of the PS20 by hydrolytic mechanism. The degradation of PS20 was so rapid that that the PS20 was mostly degraded by the time even sample analysis could be completed. However, the new process Protein A eluate shows minimal loss of Polyester and monoester species and minimal increase in non-ester species (FIGS. 21-23).

Additionally, formation of lauric acid confirms hydrolysis as the mechanism of action. Due to poor solubility of lauric acid, accurate quantitation of lauric acid over the time course of the stability study was not possible. However, significant difference in levels of Lauric acid were observed in the old and new process. Almost 17 ppm of LA was observed in old process immediately upon spiking and <1 ppm was observed in the new process with no increase over 14 days.

The profiles below confirm that the new improved wash condition in the Protein A process and the lower loading ratio have led to high PS20 stability.

### PS20 Profile in BDS Using the New Protein A Process

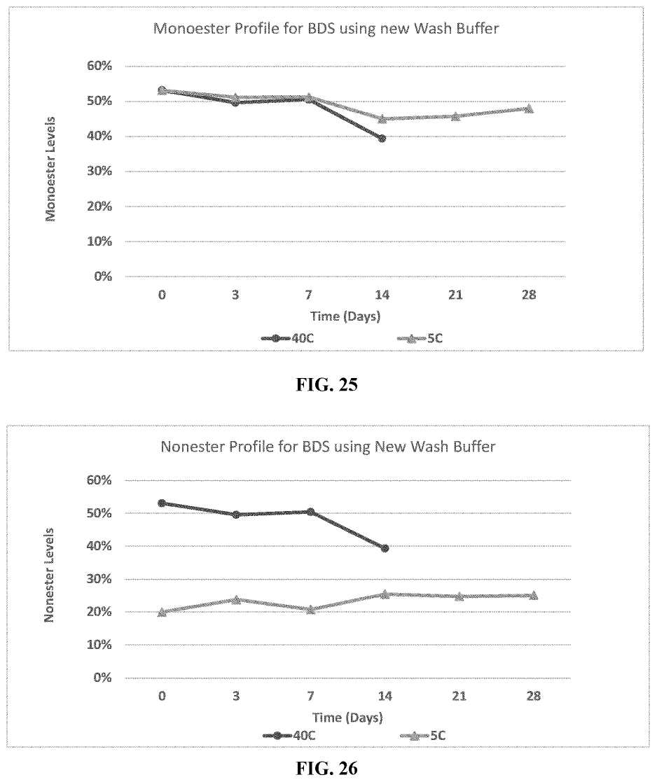

The BDS generated using the new Protein A capture chromatography process was further evaluated for PS20 stability using LC/MS. A PS20 kinetic study at 5° C. and 40° C. was performed over a period of 28 days and 14 days, respectively. As shown in FIGS. 24-27, stable polyester (FIG. 24) and monoester (FIG. 25) profiles are observed even at highly accelerated temperature of 40° C., when compared to 5° C.

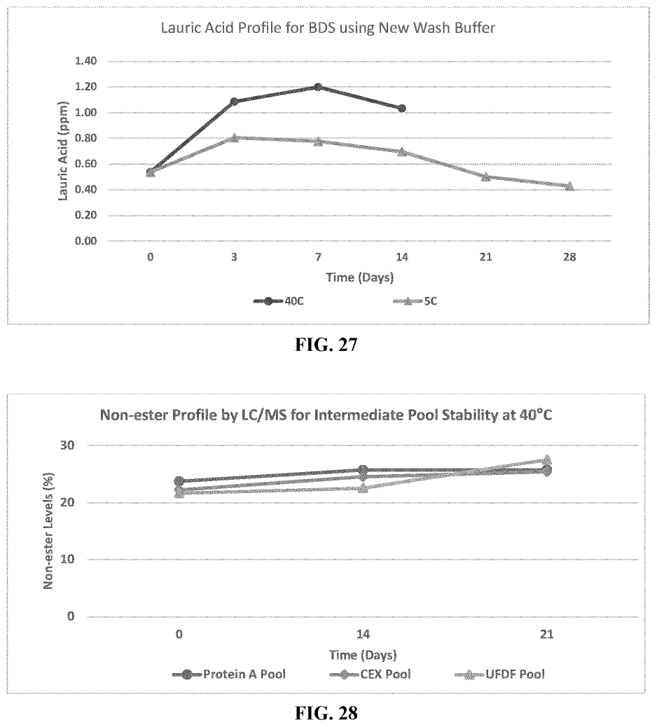

No increase in non-esterified PS20 was observed at 5° C. for 28 days and at 40° C., an increase in non-ester profile was observed (FIG. 26). Some degradation of PS20 at highly accelerated temperature over long term is expected. No increase in Lauric Acid levels was observed at 5° C. for 28 days. However, some increase was observed at 40° C. (FIG. 27).

Thus, stability of PS20 in the BDS was confirmed by orthogonal method LC/MS. These process changes were confirmed at large scale and demonstrated similar results, as discussed in section [0258].

### Host Cell Line and Related Host Cell Proteins Impacting PS20 Hydrolysis

The host cell line used for the productions of IMVT-1401 is CHO-based.

As shown above, the rate of degradation is so rapid that the PS20 was degraded immediately upon formulation or at T=0 of the kinetic study. It is not confirmed which host cell proteins specifically are responsible for the hydrolysis, or if additional factors (e.g., trace metals) may be contributing to the rapid degradation. Additionally, the quantification of HCP was performed by a generic kit that provides total HCP quantification, and not quantification of the specific HCP that may be responsible for PS20 degradation.

### pH Range for Degradation Activity

Protein A eluate was at the pH of 3.8-4.1. The samples were neutralized to pH 7 prior to testing and no difference was found in degradation pattern. Hence the HCP or the impurity was able to act on a wide range of pH.

### Formulation Independent

Given that the impurity was active at all intermediate steps, Protein A eluate, CEX Eluate, UFDF Pool and Final BDS, it is concluded that the issue is formulation independent.

### Antibody Characteristics

IMVT-1401 is a fully human monoclonal antibody in the form of immunoglobin G1 (IgG1) with two γ heavy chains and two λ light chains, linked by disulfide bonds. Given that the problematic HCP was co-eluting with IMVT-1401 in the old process at key purification steps, and it required a harsh wash 2 condition to minimize the HCP's impact in the new process, it is possible that the HCP may be interacting with IMVT-1401 and/or that certain characteristic of IMVT-1401 may preferentially bind to select subset of HCP or to the resin, enabling the host cell protein to coelute (see Li Xuanwen et al. Antib Ther. 2022 5:42-54).

### Example 2: Large Scale Confirmation Batch Using the New Protein A Chromatography Process

Protein A Chromatography process development and characterization was performed using a qualified scale down model, followed by integration of new Protein A Chromatography process with the remaining downstream process steps at bench scale in a confirmation run as described above The bench scale process was further scaled up for large scale manufacturing. The overall process schematic is shown in FIG. 2A and FIG. 2B.

PS20 stability of the scaled-up process intermediate process pools was evaluated at accelerated temperature of 40° C. The UFDF pool was evaluated at additional temperature of 2-8° C. PS20 was added to the process intermediates (Protein A eluate, AEX Pool, CEX Pool and UFDF Pool) at target concentration of 200 μg/mL, samples were held at the study temperature for the duration of the study and evaluated at the specific timepoints per the study. Results of the PS20 kinetic study are shown in Table 17. As shown, by Day 21 the Protein A eluate PS20 levels decreased by 25% when compared to initial concentration (Day 0). For the AEX pool, CEX pool and UFDF pool, PS20 levels decreased by ≤9% when compared to initial concentration (Day 0). Additionally, no difference in PS20 stability was observed for UFDF pool samples at 2-8° C. and accelerated temperature of 40° C.

Overall variability of the PS20 analytical method is expected to be almost 20%, with additional variability contributed due to sample preparation. Therefore, the stability of the PS20 at various intermediate pools from Protein A pool to UFDF pool is considered highly similar. Hence, the effectiveness of the new Protein A process at large scale for removal of problematic impurities was confirmed.

TABLE 17
PS20 stability at intermediate steps at various hold temperatures

| Condition | | PS20 Content (μg/mL) | | | | | |
|---|---|---|---|---|---|---|---|
| Process | Hold | Day | Day | Day | Day | Day | Day |
| Step/Sample | Temperature | 0 | 3 | 7 | 14 | 21 | 30 |
| Protein A Pool | 40° C. | 208 | 183 | 198 | 151 | 155 | N/A |
| AEX Pool | 40° C. | 203 | 194 | 226 | 192 | 191 | N/A |
| CEX Pool | 40° C. | 203 | 216 | 196 | 204 | 192 | N/A |
| UFDF Pool | 40° C. | 173 | 158 | 143 | 173 | 162 | N/A |
| UFDF Pool | 2-8° C. | 173 | 159 | 152 | 167 | 162 | 156 |

The UFDF pool was formulated at the target PS20 concentration of 200 μg/mL and filtered through a 0.2 μm filter to generate final BDS. PS20 stability at the BDS stage was evaluated at three temperatures; storage (−70° C.), accelerated (2-8° C.), and stressed (25° C.) for one (1) month as shown in Table 18. No loss of PS20 was observed over 1 month at the evaluated temperatures.

TABLE 18
PS20 Stability of Large Scale BDS at various hold temperatures

PS20 Content (μg/mL)

| Sample | Temperature | | T = 0 | T = 1 month |
|---|---|---|---|---|
| Drug | −70° | C. | 192 | 199 |
| Substance | 2-8° | C. | | 193 |
| | 25° | C. | | 194 |

### Example 3: PS20 Profile Characterization by Liquid Chromatography/Mass Spectrometry

LC/MS is effective to monitor the hydrolysis of PS20, as the polyesters will hydrolytically decay to monoesters and non-esterified species, modifying the percent distribution of the measured ester peaks.

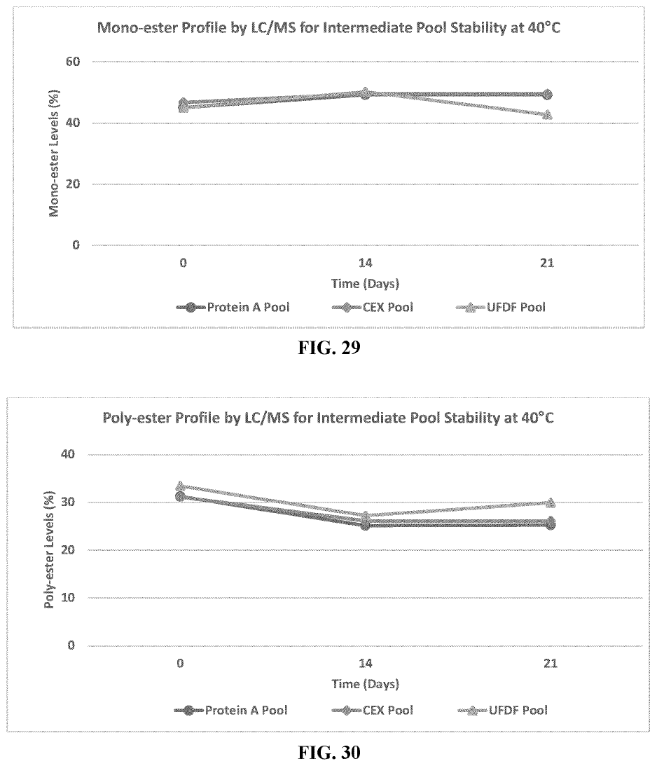

To further confirm the PS20 stability in intermediate process pools, a spiking study as described above was performed. Intermediate process pools were spiked with PS20 at the target concentration PS20 was added to the process intermediates (Protein A eluate, AEX Pool, CEX Pool and UFDF Pool) at target concentration of 200 μg/mL. Samples were held at accelerated temperature of 40° C. for the duration of the study and evaluated at the specific timepoints per study. The relative abundance of the non-ester species and polyester species in percentage area is shown in FIG. 28, FIG. 29, FIG. 30.

No practical increase in non-esterified species or a downward trend of poly-ester and mono-ester species at accelerated temperature of 40° C. up to 21 days was found for Protein A pool, CEX pool and UFDF pool, Hence, no evidence of PS20 degradation by hydrolysis was observed for any intermediate pool confirming successful scale-up of the new improved Protein A process. As no difference in non-esterified species or overall percent distribution of ester species was observed between the Protein A pool, CEX pool and UFDF pool, the profiles confirm that the problematic impurities responsible for PS20 degradation were removed using the improved Wash 2 buffer and Protein A Chromatography conditions.

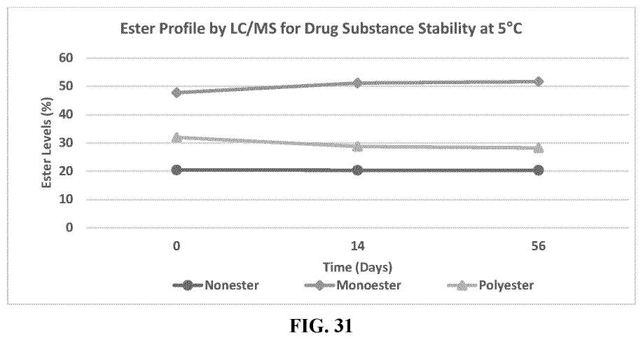

To further confirm the PS20 stability in final Bulk Drug Substance similar to [0256], BDS samples were held at accelerated temperature of 5° C. for the duration of the study and evaluated at the specific timepoints per study. The relative abundance of the non-ester species, and polyester species in percentage area is shown in FIG. 31.

No practical increase in non-esterified species or a downward trend of polyester and mono-ester species at accelerated temperature of 5° C. up to 56 days was observed. Hence, no evidence of PS20 degradation by hydrolysis was observed at BDS stage further confirming effective removal of problematic impurities upon scale-up and subsequent improved PS20 stability.

### CONCLUSIONS

The data presented herein illustrate unique ways to achieve polysorbate (e.g., PS20) formulation stability at drug substance stage without significant process changes and impacting key product quality attributes. Four main approaches were evaluated 1) increasing the pH of the Wash 2 buffer for impurity removal; 2) increasing the stringency of the wash buffer using higher concentration of Sodium Chloride; 3) Use of Arginine for Host Cell Protein solubilization 4) Evaluating operational parameters of chromatography like column load ratio.

In the experiments described above, neither the high salt nor added Arginine were required to aid in the removal of the Host Cell Proteins that cause the hydrolysis of PS20. Improved PS20 stability was achieved by using a Wash 2 buffer at higher pH and lowering the column load ratio to improve the removal of problematic host cell proteins.

Systematic screening, range finding, and confirmation studies were performed to evaluate one factor at a time and potential interaction between the various factors.

In conclusion:
1) At lower pH, Arginine when added to the Wash 2 buffer had some impact on HCP removal but no impact on the PS20 stability was observed in Protein A Eluate.
2) No additional impact of Arginine was observed at High pH in removal of problematic host cell proteins responsible for PS20 degradation.
3) pH less than 9 did not have any impact on PS20 stability
4) pH≥10 had the dominant effect and improved PS20 stability by several fold
5) High Salt (up to 1M NaCl) had no impact on PS20 stability below pH 9
6) Problematic HCP can be removed or significantly reduced at Protein A step using high pH wash buffer and subsequently reduce impurity load on remaining chromatography step.
7) Lowering the column load ratio (operational parameter) at Protein A Chromatography, along with the Wash 2 buffer pH, improves PS20 stability.

PS20 stability is observed at low concentrations of monoclonal antibody, however, often shows instability at high product concentration (e.g., >50 g/L or >150 g/L). When the high pH wash was integrated with the overall purification process it allowed concentration of the monoclonal antibody to a high concentration (e.g., >50 g/L or >150 g/L).

To summarize, higher pH without the added salt or Arginine was sufficient to allow the separation of the HCP from the Protein A Capture Eluate without impact on the stability of the monoclonal antibody or a significant reduction in process yield. Additionally, the use of the process described will allow higher protein concentrations and potentially higher polysorbate (e.g., PS20 and PS80) concentrations to be used without concern for degradation of these polyesters by a residual HCP with lipase or esterase like activity.

These process changes were confirmed at large scale and demonstrated effective removal of residual HCP with lipase or esterase like activity, resulting in stable surfactant (PS20) profile.

### INCORPORATION BY REFERENCE

All patents, patent applications and publications (e.g., scientific publications) cited herein are hereby incorporated by reference herein in their entirety for all purposes.

What is claimed is:

1. A formulation comprising (i) a protein having an Fc region at a concentration of higher than 50 g/L, and (ii) one or more excipients comprising a polysorbate, wherein the concentration of the polysorbate remains within 30% of the starting concentration of the polysorbate after storage for a period of time of at least 1 day and/or wherein the presence of a polysorbate degradation product does not increase by more than 2 times of the starting concentration of the degradation product after storage for a period of time of at least 1 day.

2. The formulation of claim 1, wherein the concentration of higher than 50 mg/ml is a concentration equal to or higher than 100 g/L.

3. The formulation of claim 1, wherein the concentration of higher than 100 mg/ml is a concentration equal to or higher than 150 g/L, 170 g/L, or 200 g/L.

4. The formulation of any one of claims 1-3, wherein the protein having an Fc region is an antibody.

5. The formulation of claim 4, wherein the antibody is a humanized or fully human monoclonal antibody.

6. The formulation of claim 4 or claim 5, wherein the antibody is an IgG1 antibody.

7. The formulation of any one of claims 4-6, wherein the antibody is an anti-FcRn antibody that specifically binds to FcRn.

8. The formulation of claim 7, wherein the anti-FcRn antibody is a fully human monoclonal IgG1 antibody.

9. The formulation of claim 7 or 8, wherein the anti-FcRn antibody comprises
(a) (i) three heavy chain CDR amino acid sequences of SEQ ID No: 27 (HCDR1), SEQ ID No: 28 (HCDR2), SEQ ID No: 29 (HCDR3); and (ii) three light chain CDR amino acid sequences of SEQ ID No: 30 (LCDR1), SEQ ID No: 31 (LCDR2), SEQ ID No: 32 (LCDR3); or
(b) (i) three heavy chain CDR amino acid sequences of SEQ ID No: 49 (HCDR1), SEQ ID No: 22 (HCDR2), SEQ ID No: 23 (HCDR3); and (ii) three light chain CDR amino acid sequences of SEQ ID No: 50 (LCDR1), SEQ ID No: 25 (LCDR2), SEQ ID No: 26 (LCDR3).

10. The formulation of any one of claims 7-9, wherein the anti-FcRn antibody comprises
(a) a heavy chain variable region amino acid sequence of SEQ ID No: 6 and a light chain variable region amino acid sequence of SEQ ID No: 16; or
(b) a heavy chain amino acid sequence of SEQ ID No: 51; and a light chain amino acid sequence of SEQ ID No: 52.

11. The formulation if any one of claims 1-10, wherein the polysorbate is Polysorbate 20.

12. The formulation if any one of claims 1-10, wherein the polysorbate is Polysorbate 80.

13. The formulation of any one of claims 1-12, wherein the polysorbate is at a concentration from about 0.002% to 0.2%.

14. The formulation of any one of claims 1-12, wherein the polysorbate is at a concentration from 0.01 to 0.05%.

15. The formulation of any one of claims 7-10, wherein the pharmaceutical formulation comprises at least or about 170 g/L of the anti-FcRn antibody, 0.02% Polysorbate 20, 100 mM L-Histidine/Histidine HCl and 100 mM L-Arginine HCl in water at pH 6.0.

16. The formulation of any one of claims 1-15, wherein the concentration of the polysorbate remains within 20% of the starting concentration of the polysorbate after storage.

17. The formulation of any one of claims 1-15, wherein the concentration of the polysorbate remains within 10% of the starting concentration of the polysorbate after storage.

18. The formulation of any one of claims 1-15, wherein the concentration of the polysorbate remains within 5% of the starting concentration of the polysorbate after storage.

19. The formulation of any one of claims 1-18, wherein the polysorbate is Polysorbate 20, and the degradation product is a non-esterified species of Polysorbate 20.

20. The formulation of claim 19, wherein the non-esterified species of Polysorbate 20 is Sorbitan or Isosorbide.

21. The formulation of any one of claims 1-18, wherein the polysorbate is Polysorbate 20, and Polysorbate 20 degradation product is Lauric acid.

22. The formulation of any one of claims 1-18, wherein the polysorbate is Polysorbate 80, and Polysorbate 80 degradation product is Oleic acid.

23. The formulation of any one of claims 1-22, wherein the presence of a polysorbate degradation product does not increase by more than 50% of the starting concentration of the degradation product after storage.

24. The formulation of any one of claims 1-22, wherein the presence of a polysorbate degradation product does not increase by more than 25% of the starting concentration of the degradation product after storage.

25. The formulation of any one of claims 1-22, wherein the presence of a polysorbate degradation product does not increase by more than 10% of the starting concentration of the degradation product after storage.

26. The formulation of any one of claims 1-25, wherein the concentration of polyester species of the polysorbate remain within 10% or 20% of the starting concentration of the polyester species after storage for a period of time of at least 1 day.

27. The formulation of any one of claims 1-25, wherein the concentration of monoester species of the polysorbate remain within 10% or 20% of the starting concentration of the monoester species after storage for a period of time of at least 1 day.

28. The formulation of any one of claims 1-27, wherein storage for a period of time of at least 1 day is storage for 1, 3 or 7 days.

29. The formulation of any one of claims 1-27, wherein storage for a period of time of at least 1 day is storage for about or more than 14 or 28 days.

30. The formulation of any one of claims 1-27, wherein storage for a period of time of at least 1 days is storge for about or more than 8 weeks or 12 weeks.

31. The formulation of any one of claims 1-27, wherein storage for a period of time of at least 1 days is storage for about or more than 1 year.

32. The formulation of any one of claims 1-31, wherein the storage is at 2-8° C.

33. The formulation of any one of claims 1-31, wherein the storage is at 25° C.

34. The formulation of any one of claims 1-31, wherein the storage is at 40° C.

35. The formulation of any one of claims 1-34, which has no or substantially no cell culture host cell proteins having lipase and/or hydrolase activity.

36. The formulation of any one of claims 1-35, wherein the formulation is a pharmaceutical formulation, and wherein the one or more excipients are pharmaceutically acceptable.

37. The formulation of any one of claims 1-36, wherein the protein having an Fc region has been purified from cell culture by an Fc-binding affinity resin chromatography.

38. The formulation of claim 37, wherein the Fc-binding affinity resin chromatography is a Protein A chromatography column or a Protein G chromatography column.

39. A method of purifying a protein having an Fc region from harvested cell culture, the method comprising:
(i) loading the harvested cell culture comprising the protein having an Fc region on to an Fc-binding affinity resin chromatography column,
(ii) washing the column one, two, three or more times using one or more wash buffers, wherein at least one wash buffer comprises a pH of greater than 9, and
(iii) eluting the protein having an Fc region from the column;
optionally, wherein the method is for making a high concentration formulation of the protein with one or more excipients comprising a polysorbate.

40. The method of claim 39, comprising, after step (iii), formulating the protein from step (iii) into a formulation comprising adding one or more excipients, wherein the concentration of the protein in the formulation is higher than 50 g/L, and wherein the one or more excipients comprise a polysorbate.

41. The method of claim 39 or 40, wherein the polysorbate is Polysorbate 20.

42. The method of claim 39 or 40, wherein the polysorbate is Polysorbate 80.

43. The method of any one of claims 39-42, wherein the polysorbate in the formulation is at a concentration from about 0.002% to 0.2%.

44. The method of any one of claims 39-42, wherein the polysorbate in the formulation is at a concentration from 0.01 to 0.05%.

45. The method of any one of claims 39-44, wherein the Fc-binding affinity resin chromatography column is a Protein A chromatography column.

46. The method of any one of claims 39-44, wherein the Fc-binding affinity resin chromatography column is a Protein G chromatography column.

47. The method of any one of claims 39-46, wherein the Fc-binding affinity resin chromatography column load ratio in step (i) is 35 g/L or less.

48. The method of any one of claims 39-46, wherein the Fc-binding affinity resin chromatography column load ratio in step (i) is 40 g/L or less.

49. The method of any one of claims 39-48, wherein the at least one wash buffer comprises a pH of at least or more than 10.

50. The method of any one of claims 39-48, wherein the at least one wash buffer comprises a pH of at least or more than 10.5, or about 10.5.

51. The method of any one of claims 39-50, wherein the at least one wash buffer comprises 1M Sodium Chloride or higher concentration of Sodium Chloride.

52. The method of any one of claims 39-50, wherein the at least one wash buffer comprises concentration of Sodium Chloride that is less than 1M or less than 0.5M.

53. The method of any one of claims 39-52, wherein the at least one wash buffer comprises arginine.

54. The method of any one of claims 39-52, wherein the at least one wash buffer does not comprise arginine.

55. The method of any one of claims 39-54, wherein the at least one wash buffer comprises sodium phosphate or sodium carbonate.

56. The method of claim 55, wherein the at least one wash buffer comprises 50 mM sodium carbonate.

57. The method of any one of claims 39-56, wherein the concentration of the protein having an Fc region in the formulation is about or higher than 100 g/L.

58. The method of any one of claims 39-57, wherein the concentration of the protein having an Fc region in the formulation is about or higher than 150 g/L, 170 g/L, or 200 g/L.

59. The method of any one of claims 39-58, wherein the formulation is a pharmaceutical formulation.

60. The method of any one of claims 39-59, wherein the protein having an Fc region is an antibody.

61. The method of claim 60, wherein the antibody is a humanized or human monoclonal antibody.

62. The method of claim 60 or 61, wherein the antibody is an IgG1 antibody.

63. The method of any one of claims 60-62, wherein the antibody is an anti-FcRn antibody that specifically binds to FcRn.

64. The method of claim 63, wherein the anti-FcRn antibody is a fully human monoclonal IgG1 antibody.

65. The method of claim 63 or 64, wherein the anti-FcRn antibody comprises (a) (i) three heavy chain CDR amino acid sequences of SEQ ID No: 27 (HCDR1), SEQ ID No: 28 (HCDR2), SEQ ID No: 29 (HCDR3); and (ii) three light chain CDR amino acid sequences of SEQ ID No: 30 (LCDR1), SEQ ID No: 31 (LCDR2), SEQ ID No: 32 (LCDR3); or
(b) (i) three heavy chain CDR amino acid sequences of SEQ ID No: 49 (HCDR1), SEQ ID No: 22 (HCDR2), SEQ ID No: 23 (HCDR3); and (ii) three light chain CDR amino acid sequences of SEQ ID No: 50 (LCDR1), SEQ ID No: 25 (LCDR2), SEQ ID No: 26 (LCDR3).

66. The method of any one of claims 63-65, wherein the anti-FcRn antibody comprises
(a) a heavy chain variable region amino acid sequence of SEQ ID No: 6 and a light chain variable region amino acid sequence of SEQ ID No: 16; or
(b) a heavy chain amino acid sequence of SEQ ID No: 51; and a light chain amino acid sequence of SEQ ID No: 52.

67. The method of any one of claims 39-66, wherein the harvested cell culture is a cell culture fluid harvested from host cells used for production of the protein having an Fc region, optionally wherein the production is by introducing a vector comprising the protein having an Fc region into the host cells and culturing the host cells under conditions suitable for expression of the protein, optionally wherein the host cells are mammalian cells suitable for the production.

68. The method of claim 66, wherein the host cells are CHO cells.

69. The method of claim 67 or 68, wherein the host cells are harvested by centrifugation, optionally followed by filtration.

70. The method of any one of claims 39-69, wherein, after step (iii), the eluate, comprising the protein having an Fc region, is subjected to filtration.

71. The method of any one of claims 39-70, wherein, after step (iii), the eluate, comprising the protein having an Fc region, is subjected to low pH viral inactivation.

72. The method of any one of claims 39-71, wherein, the eluate, comprising the protein having an Fc region, is subjected to anion exchange (AEX) chromatography and/or cation exchange (CEX) chromatography.

73. The method of any one of claims 39-72, wherein, after step (iii) and optionally after one, two, three or all of the steps specified in claims 70-72, the eluate, comprising the protein having an Fc region, is subjected to concentration.

74. The method of any one of claims 39-73, wherein the protein having an Fc region is an anti-FcRn antibody that specifically binds to FcRn, and wherein the formulation is a pharmaceutical formulation comprising at least or about 170 g/L of the anti-FcRn antibody, 0.02% Polysorbate 20, 100 mM L-Histidine/Histidine HCl and 100 mM L-Arginine HCl in water at pH 6.0.

75. A formulation produced using the method of any one of claims 39-74.

76. The formulation of claim 75, wherein the polysorbate comprises Polysorbate 20 (PS20) or Polysorbate 80 (PS80), and wherein the concentration of the PS20 or PS80 remains within 30% of the starting concentration of the PS20 or PS80 after storage for a period of time of at least 1 day and/or wherein the presence of a PS20 or PS80 degradation product does not increase by more than 2 times of the starting concentration of the degradation product after storage for a period of time of at least 1 day.

77. The formulation of claim 76, wherein the concentration of the PS20 or PS80 remains after storage within 20% of the starting concentration of the PS20 or PS80.

78. The formulation of claim 76, wherein the concentration of the PS20 or PS80 remains after storage within 10% of the starting concentration of the PS20 or PS80.

79. The formulation of claim 76, wherein the concentration of the PS20 or PS80 remains after storage within 5% of the starting concentration of the PS20 or PS80.

80. The formulation of any one of claims 75-79, wherein the PS20 or PS80 degradation product is a fatty acid or its ester.

81. The formulation of any one of claims 75-79, wherein the PS20 degradation product is a non-esterified species of PS20.

82. The formulation of claim 81, wherein the non-esterified species of PS20 is Sorbitan or Isosorbide.

83. The formulation of any one of claims 75-79, wherein the PS20 degradation product is Lauric acid.

84. The formulation of any one of claims 75-79, wherein the PS80 degradation product is Oleic acid.

85. The formulation of any one of claims 75-84, wherein the presence of a PS20 or PS80 degradation product does not increase by more than 50% of the starting concentration of the degradation product after storage.

86. The formulation of any one of claims 75-84, wherein the presence of a PS20 or PS80 degradation product does not increase by more than 25% of the starting concentration of the degradation product after storage.

87. The formulation of any one of claims 75-84, wherein the presence of a PS20 or PS80 degradation product does not increase by more than 10% of the starting concentration of the degradation product after storage.

88. The formulation of any one of claims 75-84, wherein the concentration of polyester species of PS20 or PS80 remain within 10% or 20% of the starting concentration of the polyester species after storage for a period of time of at least 1 day.

89. The formulation of any one of claims 75-84, wherein the concentration of monoester species of PS20 or PS80 remain within 10% or 20% of the starting concentration of the monoester species after storage for a period of time of at least 1 day.

90. The formulation of any one of claims 75-89, wherein storage for a period of time of at least 1 day is storage for 1, 3 or 7 days.

91. The formulation of any one of claims 75-89, wherein storage for a period of time of at least 1 day is storage for about or more than 14 or 28 days.

92. The formulation of any one of claims 75-89, wherein storage for a period of time of at least 1 days is storge for about or more than 8 weeks or 12 weeks.

93. The formulation of any one of claims 75-89, wherein storage for a period of time of at least 1 days is storage for about or more than 1 year.

94. The formulation of any one of claims 75-93, wherein the storage is at 2-8° C.

95. The formulation of any one of claims 75-93, wherein the storage is at 25° C.

96. The formulation of any one of claims 75-93, wherein the storage is at 40° C.

97. The formulation of any one of claims 75-96, wherein the formulation has no or substantially no cell culture host cell proteins having lipase, esterase and/or hydrolase activity.
